SEQUENCE LISTING (28 sequences)

SEQ ID NO: 1 - protein, 19 aa, synthetic construct
MEFGLSWLFLVAILKGVQC

SEQ ID NO: 2 - protein, 10 aa, synthetic construct
EQKLISEEDL

SEQ ID NO: 3 - protein, 745 aa, synthetic construct
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 4 - protein, 10 aa, synthetic construct
RVETATETAW

SEQ ID NO: 5 - protein, 22 aa, synthetic construct
ISLVTALHLVLGLSAVLGLLLL

SEQ ID NO: 6 - protein, 122 aa, synthetic construct
RWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 7 - protein, 928 aa, synthetic construct
MEFGLSWLFLVAILKGVQCEQKLISEEDLASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLERVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 8 - protein, 505 aa, synthetic construct
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 9 - protein, 688 aa, synthetic construct
MEFGLSWLFLVAILKGVQCEQKLISEEDLASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLERVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 10 - protein, 319 aa, synthetic construct
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKIEGAQEKTNLE

SEQ ID NO: 11 - protein, 502 aa, synthetic construct
MEFGLSWLFLVAILKGVQCEQKLISEEDLASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKIEGAQEKTNLERVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 12 - DNA, 2787 bp, synthetic construct
  source 1..2787: mol_type = other DNA
atggaattcggcctgagctggctgttcctggtggccatcctgaagggcgtgcagtgcgagcagaagctgatctccgaagaggacctggccagcgtgggactgcctagcgtgtcactggacctgcccagactgagcatccagaaggacatcctgaccatcaaggccaacaccaccctgcagatcacctgtcggggccagagggacctggattggctgtggcccaacaaccagagcggcagcgagcagagggtggaagtgaccgagtgtagcgacggcctgttctgcaagaccctgacaatccccaaagtgatcggcaacgacaccggcgcctacaagtgcttctacagagagacagacctggcctccgtgatctacgtgtacgtgcaggactacagaagcccctttatcgccagcgtgtccgaccagcacggcgtggtgtacatcaccgagaacaagaacaagaccgtcgtgatcccctgcctgggcagcatcagcaacctgaacgtgtccctgtgcgccagataccccgagaagagattcgtgcccgacggcaaccggatcagctgggacagcaagaagggcttcaccatccccagctacatgatcagctacgccggcatggtgttctgcgaggccaagatcaacgacgagagctaccagagcatcatgtacatcgtcgtggtcgtgggctaccggatctacgacgtggtgctgagccctagccacggcatcgagctgtctgtgggcgagaagctggtgctgaactgcaccgccagaaccgagctgaacgtgggcatcgacttcaactgggagtaccccagcagcaagcaccagcacaagaaactcgtgaaccgggacctgaaaacccagtccggctccgagatgaagaagttcctgagcacactgaccatcgacggcgtgaccagaagcgaccagggcctgtacacatgtgccgccagcagcggcctgatgaccaagaaaaacagcaccttcgtgcgggtgcacgagaagcctttcgtggcctttggcagcggcatggaaagcctggtggaagccaccgtgggagagagagtgcggatccccgccaagtacctgggctaccctccacccgagatcaagtggtacaagaacggcatccccctggaaagcaaccacacaatcaaggccggccacgtgctgacaatcatggaagtgtctgagcgggacaccggcaattacaccgtgatcctgactaaccccatcagcaaagaaaagcagagccacgtggtgtccctggtggtgtatgtgcccccccagatcggcgagaagtccctgatcagccccgtggacagctaccagtacggcaccacccagacactgacctgcaccgtgtacgccatccctccccctcaccacatccactggtactggcagctggaagaggaatgcgccaacgagcctagccaggccgtgtccgtgaccaacccctacccttgtgaagagtggcggagcgtggaagatttccagggcggcaacaagatcgaagtgaacaagaatcagttcgccctgatcgagggcaaaaacaagacagtgtccaccctcgtgatccaggccgccaatgtgtccgccctgtacaagtgcgaggccgtgaacaaagtgggcagaggcgagagagtgatctccttccacgtgacccggggaccagagatcacactgcagcccgacatgcagcccaccgagcaggaatccgtgtctctgtggtgcaccgccgaccggtccaccttcgagaacctgacctggtacaagctgggcccccagcccctgcctatccacgtgggagaactgcctacccccgtgtgcaagaacctggacaccctgtggaagctgaacgccaccatgttcagcaacagcaccaacgatatcctgatcatggaactgaagaacgccagcctgcaggaccagggcgactacgtgtgtctggcccaggacagaaagaccaaaaagcggcactgcgtcgtgcggcagctgaccgtgctggaaagagtggcccctaccatcaccggcaacctggaaaaccagaccacctccatcggcgagtccatcgaggtgtcctgtaccgccagcggcaaccccccacctcagatcatgtggtttaaggacaacgagacactggtggaagatagcggcatcgtgctgaaggacggcaacagaaatctgaccattcgcagagtgcgcaaagaggacgagggactgtacacctgtcaggcctgtagcgtgctgggctgcgccaaagtggaagccttctttatcatcgagggcgcccaggaaaagaccaatctggaaagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 13 - DNA, 2067 bp, synthetic construct
  source 1..2067: mol_type = other DNA
atggaattcggcctgagctggctgttcctggtggccatcctgaagggcgtgcagtgcgagcagaagctgatctccgaagaggacctggccagcgtgggactgcctagcgtgtcactggacctgcccagactgagcatccagaaggacatcctgaccatcaaggccaacaccaccctgcagatcacctgtcggggccagagggacctggattggctgtggcccaacaaccagagcggcagcgagcagagggtggaagtgaccgagtgtagcgacggcctgttctgcaagaccctgacaatccccaaagtgatcggcaacgacaccggcgcctacaagtgcttctacagagagacagacctggcctccgtgatctacgtgtacgtgcaggactacagaagcccctttatcgccagcgtgtccgaccagcacggcgtggtgtacatcaccgagaacaagaacaagaccgtcgtgatcccctgcctgggcagcatcagcaacctgaacgtgtccctgtgcgccagataccccgagaagagattcgtgcccgacggcaaccggatcagctgggacagcaagaagggcttcaccatccccagctacatgatcagctacgccggcatggtgttctgcgaggccaagatcaacgacgagagctaccagagcatcatgtacatcgtcgtggtcgtgggctaccggatctacgacgtggtgctgagccctagccacggcatcgagctgtctgtgggcgagaagctggtgctgaactgcaccgccagaaccgagctgaacgtgggcatcgacttcaactgggagtaccccagcagcaagcaccagcacaagaaactcgtgaaccgggacctgaaaacccagtccggctccgagatgaagaagttcctgagcacactgaccatcgacggcgtgaccagaagcgaccagggcctgtacacatgtgccgccagcagcggcctgatgaccaagaaaaacagcaccttcgtgcgggtgcacgagaagcctttcgtggcctttggcagcggcatggaaagcctggtggaagccaccgtgggagagagagtgcggatccccgccaagtacctgggctaccctccacccgagatcaagtggtacaagaacggcatccccctggaaagcaaccacacaatcaaggccggccacgtgctgacaatcatggaagtgtctgagcgggacaccggcaattacaccgtgatcctgactaaccccatcagcaaagaaaagcagagccacgtggtgtccctggtggtgtatgtgcccgtgctggaaagagtggcccctaccatcaccggcaacctggaaaaccagaccacctccatcggcgagtccatcgaggtgtcctgtaccgccagcggcaaccccccacctcagatcatgtggtttaaggacaacgagacactggtggaagatagcggcatcgtgctgaaggacggcaacagaaatctgaccattcgcagagtgcgcaaagaggacgagggactgtacacctgtcaggcctgtagcgtgctgggctgcgccaaagtggaagccttctttatcatcgagggcgcccaggaaaagaccaatctggaaagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 14 - DNA, 1509 bp, synthetic construct
  source 1..1509: mol_type = other DNA
atggaattcggcctgagctggctgttcctggtggccatcctgaagggcgtgcagtgcgagcagaagctgatctccgaagaggacctggccagcgtgggactgcctagcgtgtcactggacctgcccagactgagcatccagaaggacatcctgaccatcaaggccaacaccaccctgcagatcacctgtcggggccagagggacctggattggctgtggcccaacaaccagagcggcagcgagcagagggtggaagtgaccgagtgtagcgacggcctgttctgcaagaccctgacaatccccaaagtgatcggcaacgacaccggcgcctacaagtgcttctacagagagacagacctggcctccgtgatctacgtgtacgtgcaggactacagaagcccctttatcgccagcgtgtccgaccagcacggcgtggtgtacatcaccgagaacaagaacaagaccgtcgtgatcccctgcctgggcagcatcagcaacctgaacgtgtccctgtgcgccagataccccgagaagagattcgtgcccgacggcaaccggatcagctgggacagcaagaagggcttcaccatccccagctacatgatcagctacgccggcatggtgttctgcgaggccaagatcaacgacgagagctaccagagcatcatgtacatcgtcgtggtcgtgggctaccggatctacgacgtggtgctgagccctagccacggcatcgagctgtctgtgggcgagaagctggtgctgaactgcaccgccagaaccgagctgaacgtgggcatcgacttcaactgggagtaccccagcagcaagcaccagcacaagaaactcgtgaaccgggacctgaaaacccagtccggctccgagatgaagaagttcctgagcacactgaccatcgacggcgtgaccagaagcgaccagggcctgtacacatgtgccgccagcagcggcctgatgaccaagaaaaacagcaccttcgtgcgggtgcacgagaagatcgagggcgcccaggaaaagaccaatctggaaagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 15 - protein, 498 aa, synthetic construct
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

SEQ ID NO: 16 - protein, 671 aa, synthetic construct
MEFGLSWLFLVAILKGVQCIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 17 - protein, 491 aa, synthetic construct
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISPDEFLFTP

SEQ ID NO: 18 - protein, 664 aa, synthetic construct
MEFGLSWLFLVAILKGVQCIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISPDEFLFTPRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 19 - protein, 483 aa, synthetic construct
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPIS

SEQ ID NO: 20 - protein, 656 aa, synthetic construct
MEFGLSWLFLVAILKGVQCIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 21 - DNA, 2016 bp, synthetic construct
  source 1..2016: mol_type = other DNA
atggaatttggcctgagctggctgttcctggtggccatcctgaagggcgtgcagtgcatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacactaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccaagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 22 - DNA, 1995 bp, synthetic construct
  source 1..1995: mol_type = other DNA
atggaatttggcctgagctggctgttcctggtggccatcctgaagggcgtgcagtgcatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacactaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctccggatgagttcctcttcacaccaagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 23 - DNA, 1971 bp, synthetic construct
  source 1..1971: mol_type = other DNA
atggaatttggcctgagctggctgttcctggtggccatcctgaagggcgtgcagtgcatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacactaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttaa

SEQ ID NO: 24 - DNA, 18 bp, synthetic construct
  source 1..18: mol_type = other DNA
gcccctgatgcgatgcgg

SEQ ID NO: 25 - DNA, 18 bp, synthetic construct
  source 1..18: mol_type = other DNA
gagccgtggtccgcgcgg

SEQ ID NO: 26 - DNA, 18 bp, synthetic construct
  source 1..18: mol_type = other DNA
cgggctcatggacgggtg

SEQ ID NO: 27 - DNA, 18 bp, synthetic construct
  source 1..18: mol_type = other DNA
gatgttggactcctcagt

SEQ ID NO: 28 - protein, 10 aa, synthetic construct
EQKLISEEDL

(19) United States
(12) Patent Application Publication
(10) Pub. No.: US 2025/0262274 A1
(43) Pub. Date: Aug. 21, 2025

(54) Designed Biosensors for Enhanced T Cell Therapy

(71) Applicants: École Polytechnique Fédérale de Lausanne, Lausanne (CH); Centre Hospitalier Universitaire Vaudois, Lausanne (CH)

(72) Inventors: Caroline Arber, Lausanne (CH); Patrick Barth, Lausanne (CH); Jan A. Rath, Lausanne (CH); Lucas Rudden, Renens (CH)

(21) Appl. No.: 18/859,133

(22) PCT Filed: May 17, 2023
(86) PCT No.: PCT/EP2023/063385

§ 371 Date: Oct. 22, 2024

Related U.S. Application Data
(60) Provisional application No. 63/364,810, filed on May 17, 2022.
(60) Provisional application No. 63/496,453, filed on Apr. 17, 2023.

(51) Int. Cl. A61K 38/17; A61P 35/00; C07K 14/71; C07K 14/715; G16B 15/30

(52) U.S. Cl. CPC A61K 38/179; A61K 38/1793; A61P 35/00; C07K 14/71; C07K 14/7155; G16B 15/30; C07K 2319/02; C07K 2319/03

(57) ABSTRACT

This disclosure describes a method for de novo bottom-up assembly and rational design of allosteric biosensors with programmable input-output behaviors that respond to soluble factors selectively enriched in tumors and trigger co-stimulation and cytokine signals. The disclosed method of effective mechanical coupling and biosensor signaling potency correlates with anti-tumor function. This disclosure provides synthetic biosensors with custom-built sensing and responses for basic and translational cell engineering applications.

### CROSS-REFERENCE TO RELATED APPLICATIONS

This application claims priority under 35 U.S.C. § 119(e) to U.S. Provisional Patent Application No. 63/364,810, filed May 17, 2022 and U.S. Provisional Patent Application No. 63/496,453, filed Apr. 17, 2023. The foregoing applications are incorporated by reference herein in their entireties.

### FIELD OF THE INVENTION

This invention relates to biosensors for enhanced T cell therapy and methods of rational design thereof.

### BACKGROUND OF THE INVENTION

Engineered T cell immunotherapies can achieve potent anti-tumor responses against hematologic malignancies, but efficacy against solid tumors remains limited (Wagner, J., et al. Mol Ther 28, 2320-2339 (2020); Rafiq, S., et al. Nat Rev Clin Oncol 17, 147-167 (2020)). The complexity of the tumor microenvironment (ME) in different hematologic malignancies can also impact the success of engineered T cell therapies. Significant roadblocks to success include limited T cell trafficking, function, persistence, altered growth factor and immune cell composition in the TME, tumor heterogeneity, and paucity of tumor-specific antigens. In addition to antigen recognition, human therapeutic T cells require co-stimulation and cytokine signals for optimal anti-tumor activity. However, in most solid tumors, co-stimulation is dominated by co-inhibition, and cytokine signals required for sustained T cell expansion and memory formation are lacking or are overruled by immunosuppressive signals.

Thus, there remains a strong need for novel methods or agents for enhanced T cell therapy.

### SUMMARY OF THE INVENTION

This disclosure addresses the need mentioned above in a number of aspects. In one aspect, this disclosure provides a chimeric polypeptide, comprising: (i) an extracellular ligand-binding domain capable of binding to an input signal associated with cancer, (ii) an intracellular signaling domain capable of activating and/or enhancing an anti-tumor function, and (iii) a transmembrane domain linking the extracellular ligand-binding domain and the intracellular signaling domain, wherein the extracellular ligand-binding domain and the intracellular signaling domain are derived from different receptors, and wherein binding of the input signal to the extracellular ligand-binding domain induces oligomerization of the chimeric polypeptide, and activates and/or enhances the anti-tumor function.

In some embodiments, the input signal is present in a tumor microenvironment. In some embodiments, the cancer comprises a solid tumor. In some embodiments, the cancer comprises a hematologic malignancy.

In some embodiments, the input signal comprises a growth factor, cytokine, or interleukin. In some embodiments, the input signal comprises vascular endothelial growth factor (VEGF), basic fibroblast growth factor (bFGF), platelet-derived growth factor (PDGF), hepatocyte growth factor (HGF), insulin-like growth factor (IGF), interleukin-8 (IL-8), TGFβ, IL-10, or colony stimulating factor 1 (CSF-1), interleukin-34 (IL-34), stem cell factor (SCF), interleukin-9 (IL-9), interleukin-4 (IL-4), interleukin-6 (IL-6), interleukin-10 (IL-10), Angiopoietin-1 (Ang1), or CD202), Thrombopoietin (TPO), Osteopontin (OPN), Receptor activator of nuclear factor kappa beta (NFkB) ligand, RANK ligand (RANKL), Fibroblast growth factor (FGF-1, -2), Vascular cell adhesion protein 1 (VCAM-1), Notch ligands: Jagged1, Jagged2, Delta-like1, Delta-like3, Delta-like4, GM-SCF/CSF2, G-CSF/CSF3, IL-1b, MMP-1, MMP-2, MMP-3, MMP-7, MMP-8, MMP-9, MMP-13, MMP-14, TIMP-1, TIMP-2, TIMP-3, TIMP-4, PGD2, PGE2, PGF2α, PGI2, TXA2, PGH2, BMP1, BMP2, BMP3, BMP4, BMP5, BMP6, BMP7, BMP8, BMP9, BMP10, BMP11, BMP12, BMP13, BMP14, BMP15, N6-Cyclopentyladenosine, N6-3-methoxyl-4-hydroxybenzyl adenine riboside (B2), adenosine, CCPA, Certain Benzodiazepines and Barbiturates, 2′-MeCCPA, GR 79236, SDZ WAG 994, Benzyloxy-cyclopentyladenosine (BnOCPA), N6-3-methoxyl-4-hydroxybenzyl adenine riboside (B2), ATL-146e, CGS-21680, Regadenoson, 5′-N-ethylcarboxamidoadenosine, BAY 60-6583, LUF-5835, LUF-5845, 2-(1-Hexynyl)-N-methyladenosine, CF-101 (IB-MECA), 2-Cl-IB-MECA, CP-532,903, or MRS-3558.

In some embodiments, the anti-tumor function comprises: (i) secretion of one or more cytokines from an immune cell, (ii) co-stimulation of the immune cell, (iii) cell survival of the immune cell, (iv) proliferation of the immune cell, (v) migration of the immune cell, (vi) modified metabolism of the immune cell, (vii) altered differentiation status of the immune cell, (viii) functionality of the immune cell, or a combination thereof.

In some embodiments, binding of the input signal to the extracellular ligand-binding domain induces dimerization, trimerization, tetramerization, or polymerization of the chimeric polypeptide.

In some embodiments, the extracellular ligand-binding domain comprises one or more extracellular domains of VEGFR2, VEGFR1, FGFR1, PDGFR, HGFR, IGFR, IL-8R, TGFβR1, IL-10R, CSF1R, SCFR, KIT, cKIT, CD117, IL-9R, IL-4R, IL-6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-1R, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-1A, ActR-2A, ActR-2B, or a variant or fragment thereof.

In some embodiments, the extracellular ligand-binding domain comprises D1-7; D1-4 and D7; or D1-3 extracellular domains of the VEGFR2.

In some embodiments, the extracellular ligand-binding domain comprises an amino acid sequence having at least 90% sequence identity to SEQ ID NO: 3, 8, 10, 15, 17, or 19 or comprises the amino acid sequence of SEQ ID NO: 3, 8, 10, 15, 17, or 19.

In some embodiments, the transmembrane domain comprises a transmembrane domain from a protein selected from VEGFR2, VEGFR1, FGFR1, PDGFR, HGFR, IGFR, IL-R, TGFβR1, IL-10R, CSFR, SCFR, KIT, cKIT, CD117, IL-9R, IL-4R, 6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-R, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-1A, ActR-2A, ActR-2B, CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, toll-like receptor (TLR), TLR5, Myd88, lymphocyte receptor chain, IgE, IgG, CD16a, FcγRIII, FcγRH, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF1, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

In some embodiments, the transmembrane domain comprises an amino acid sequence having at least 90% sequence identity to SEQ ID NO: 5 or comprises the amino acid sequence of SEQ ID NO: 5.

In some embodiments, the intracellular signaling domain comprises an intracellular domain of a protein selected from CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16a, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF1, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

In some embodiments, the intracellular signaling domain comprises an intracellular domain of a cytokine receptor. In some embodiments, the intracellular signaling domain comprises an intracellular domain of a c-MPL receptor.

In some embodiments, the intracellular signaling domain comprises an amino acid sequence having at least 90% sequence identity to SEQ ID NO: 6 or comprises the amino acid sequence of SEQ ID NO: 6.

In some embodiments, the chimeric polypeptide further comprises a signaling peptide linked to the N-terminus of the extracellular ligand-binding domain. In some embodiments, the signaling peptide comprises an Igκ signaling peptide.

In some embodiments, the signaling peptide comprises an amino acid sequence having at least 90% sequence identity to SEQ ID NO: 1 or comprises the amino acid sequence of SEQ ID NO: 1.

In some embodiments, the transmembrane domain is linked to the extracellular ligand-binding domain via a linker. In some embodiments, the transmembrane domain is linked to the intracellular signaling domain via a linker. In some embodiments, the linker connecting the transmembrane domain and the intracellular signaling domain comprises a juxtamembrane (JM) region.

In some embodiments, the chimeric polypeptide comprises an amino acid sequence having at least 90% sequence identity to SEQ ID NO: 7, 9, 11, 16, 18, or 20 or comprises the amino acid sequence of SEQ ID NO: 7, 9, 11, 16, 18, or 20.

Also within the scope of this disclosure are a nucleic acid comprising a polynucleotide sequence that encodes a chimeric polypeptide disclosed herein, and a vector comprising the nucleic acid disclosed herein. In some embodiments, the vector comprises a viral vector. In some embodiments, the vector comprises an adeno-associated viral (AAV) vector, lentiviral vector, retroviral vector, adenoviral vector, or a non-viral plasmid vector.

In another aspect, this disclosure also provides a cell comprising a nucleic acid or a vector, as disclosed herein. In some embodiments, the cell comprises an immune cell. In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell.

In some embodiments, the immune cell expresses a T cell receptor (TCR) (e.g., transgenic or natural TCR), a chimeric antigen receptor (CAR), or other tumor targeting receptor.

In another aspect, this disclosure provides a method of preparing a modified immune cell. The method comprises: (a) obtaining a plurality of immune cells; (b) introducing a vector disclosed herein into the plurality of immune cells; (c) introducing a second vector comprising a nucleic acid sequence encoding a recombinant TCR or a CAR to obtain a plurality of modified immune cells; and (d) optionally expanding the plurality of modified immune cells in a cell culture medium.

In another aspect, this disclosure provides a composition comprising: a chimeric polypeptide, a nucleic acid, a vector, or a cell, as disclosed herein.

In some embodiments, the composition comprises an additional anti-cancer or anti-tumor agent. In some embodiments, the additional anti-tumor or anti-cancer agent is selected from taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof. In some embodiments, the additional anti-cancer or anti-tumor agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

Also within the scope of this disclosure is a kit comprising: a chimeric polypeptide, a nucleic acid, a vector, a cell, or a composition, as disclosed herein.

In another aspect, this disclosure provides a method of treating cancer in a subject in need thereof. The method comprises administering to the subject a therapeutically effective amount of a cell or a composition, as disclosed herein.

In another aspect, this disclosure provides a method of treating cancer in a subject in need thereof, comprising: (a) obtaining a plurality of immune cells; (b) introducing a vector disclosed herein into the plurality of immune cells; (c) introducing a second vector comprising a nucleic acid sequence encoding a recombinant TCR or a CAR to obtain a plurality of modified immune cells; and (d) administering to the subject a therapeutically effective amount of the plurality of modified immune cells. In some embodiments, the immune cells are autologous or allogeneic.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, leukemia, Li-Fraumeni tumors, liver cancer, lung cancer, lymphoma, melanoma, meningioma, multiple neuroendocrine type I and type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the cancer comprises a solid tumor. In some embodiments, the cancer comprises a hematologic malignancy.

In some embodiments, the method further comprises administering to the patient an additional agent or therapy. In some embodiments, the additional agent comprises an anti-tumor or anti-cancer agent. In some embodiments, the additional agent comprises a pathway targeted therapy, an immune modulating therapy, or a tumor microenvironment modulating therapy.

In some embodiments, the additional agent or therapy is administered before, after, or concurrently with the composition.

In some embodiments, the anti-tumor or anti-cancer agent is selected from taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof. In some embodiments, the anti-tumor or anti-cancer agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

In some embodiments, the additional agent comprises a pathway targeted therapy, an immune modulating therapy, or a tumor microenvironment modulating therapy.

In yet another aspect, this disclosure provides a method of constructing a chimeric receptor. The method comprises: (a) selecting an extracellular ligand-binding domain and an intracellular signaling domain for the chimeric receptor from a plurality of extracellular ligand-binding domains and intracellular signaling domains based on a predetermined input signal and output function to be coupled by the chimeric receptor; (b) selecting a transmembrane domain from a plurality of transmembrane domains for linking the extracellular ligand-binding and the intracellular signaling domain; (c) self-associating three-dimensional structures of the extracellular ligand-binding domain, the intracellular signaling domain, and the transmembrane domain by docking; (d) linking and assembling subunit structures into an ensemble of chimeric receptor structures; (e) energy minimizing the oligomeric receptor models and selecting a subset of energy-minimized oligomeric receptor models having low energy by clustering the energy-minimized oligomeric receptor models based on an energy function; (f) calculating stability of the subset of the energy-minimized oligomeric receptor models upon binding to the input signal, wherein a level of stability corresponds to a degree of ligand-induced oligomerization of the chimeric receptor, (g) calculating a level of long-range mechanical dynamic coupling between the extracellular ligand-binding domain and the intracellular signaling domain of the subset of the energy-minimized oligomeric receptor models; (h) ranking the subset of the energy-minimized oligomeric receptor models based on the level of stability thereof and the level of long-range mechanical dynamic coupling between the extracellular ligand-binding domain and the intracellular signaling domain; and (i) selecting an optimal oligomeric receptor model with specific levels of stability and/or levels of long-range mechanical dynamic coupling between the extracellular ligand-binding domain and the intracellular signaling domain.

In some embodiments, the method further comprises repeating step (a) to step (i) for a plurality of combinations of extracellular ligand-binding domains, intracellular signaling domains, and transmembrane domains; and selecting an optimal oligomeric receptor model from the plurality of combinations with specific levels of stability and/or levels of long-range mechanical dynamic coupling between the extracellular ligand-binding domain and the intracellular signaling domain.

In some embodiments, step (b) comprises de novo designing TM domains for optimal signal transduction between the extracellular domain and the intracellular signaling domain.

In some embodiments, the method further comprises, prior to step (e), de novo designing an inter-domain linker connecting the extracellular ligand binding domain and the transmembrane domain or an inter-domain linker connecting the transmembrane domain and the intracellular signaling domain. In some embodiments, the method further comprises, prior to step (e), de novo designing the transmembrane domain.

In some embodiments, step (e) comprises energy minimizing the oligomeric receptor models by a Monte Carlo Minimization with simulated annealing.

In some embodiments, step (g) comprises calculating the level of long-range mechanical dynamic coupling between the extracellular ligand-binding domain and the intracellular signaling domain using a Rosetta force field and an Elastic Network model.

In some embodiments, step (g) comprises self-associating the transmembrane domain using EFDOCK-TM.

In some embodiments, the input signal is present in a tumor microenvironment associated with cancer. In some embodiments, the cancer comprises a solid tumor. In some embodiments, the cancer comprises a hematologic malignancy.

In some embodiments, the output function comprises an anti-tumor function. In some embodiments, the anti-tumor function comprises: (i) secretion of one or more cytokines from an immune cell, (ii) co-stimulation of the immune cell, (iii) cell survival of the immune cell, (iv) proliferation of the immune cell, (v) migration of the immune cell, (vi) modified metabolism of the immune cell, (vii) altered differentiation status of the immune cell, (viii) functionality of the immune cell, or a combination thereof.

In some embodiments, the input signal comprises a soluble factor that is enriched in a tumor microenvironment. In some embodiments, the input signal comprises vascular endothelial growth factor (VEGF), basic fibroblast growth factor (bFGF), platelet-derived growth factor (PDGF), hepatocyte growth factor (HGF), insulin-like growth factor (IGF), interleukin-8 (IL-8), TGFβ, IL-10, or colony stimulating factor 1 (CSF-1), interleukin-34 (IL-34), stem cell factor (SCF), interleukin-9 (IL-9), interleukin-4 (ILA), interleukin-6 (IL-6), interleukin-10 (IL-10), Angiopoietin-1 (Ang1), or CD202), Thrombopoietin (TPO), Osteopontin (OPN), Receptor activator of nuclear factor kappa beta (NFkB) ligand, RANK ligand (RANKL), Fibroblast growth factor (FGF-1, -2), Vascular cell adhesion protein 1 (VCAM-1), Notch ligands: Jagged1, Jagged2, Delta-like1, Delta-like3, Delta-like4, GM-SCF/CSF2, G-CSF/CSF3, IL-1b, MMP-1, MMP-2, MMP-3, MMP-7, MMP-8, MMP-9, MMP-13, MMP-14, TIMP-1, TIMP-2, TIMP-3, TIMP-4, PGD2, PGE2, PGF2α, PGI2, TXA2, PGH2, BMP1, BMP2, BMP3, BMP4, BMP5, BMP6, BMP7, BMP8, BMP9, BMP10, BMP11, BMP12, BMP13, BMP14, BMP15, N6-Cyclopentyladenosine, N6-3-methoxyl-4-hydroxybenzyl adenine riboside (B2), adenosine, CCPA, Certain Benzodiazepines and Barbiturates, 2′-MeCCPA, GR 79236, SDZ WAG 994, Benzyloxy-cyclopentyladenosine (BnOCPA), N6-3-methoxyl-4-hydroxybenzyl adenine riboside (B2), ATL-146e, CGS-21680, Regadenoson, adenosine, 5′-N-ethylcarboxamidoadenosine, BAY 60-6583, adenosine, LUF-5835, LUF-5845, 2-(1-Hexynyl)-N-methyladenosine, CF-101 (IB-MECA), Adenosine, 2-Cl-IB-MECA, CP-532,903, or MRS-3558.

In some embodiments, the extracellular ligand-binding domain comprises one or more extracellular domains of VEGFR2, VEGFR1, FGFR1, PDGFR, HGFR, IGFR, IL-8R, TGFβR1, IL-10R, CSF1R, SCFRKIT, cKIT, CD117, IL-9R, IL-4R, IL-6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-1R, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-IA, ActR-2A, ActR-2B, or a variant or fragment thereof.

In some embodiments, the extracellular ligand-binding domain comprises D1-7; D1-4 and D7; or D1-3 extracellular domains of the VEGFR2.

In some embodiments, the transmembrane domain comprises a transmembrane domain from a protein selected from VEGFR2, VEGFR1, FGFR1, PDGFR, HGFR, IGFR, IL-8R, TGFβR1, IL-10R, CSF1R, SCFR, KIT, cKIT, CD117, IL-9R, IL-4R, IL-6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-IR, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-1A, ActR-2A, ActR-2B, CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRI, FcγRI, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

In some embodiments, the transmembrane domain comprises a transmembrane domain of a c-MPL receptor.

In some embodiments, the intracellular signaling domain comprises an intracellular domain of a protein selected from CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

In some embodiments, the intracellular signaling domain comprises an intracellular domain of a cytokine receptor. In some embodiments, the intracellular signaling domain comprises an intracellular domain of a c-MPL receptor.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1a, 1b, 1c, 1d, and 1e are a set of diagrams showing the concept, the design and the signaling outcome of de novo assembled biosensors in human T cells. FIG. 1a shows a schematic representation of an engineered T cell in a tumor microenvironment (TME). MDSCs: myeloid-derived suppressor cells, ECM: extracellular matrix, MΦ: macrophage, T_{regs}: regulatory T cells. FIG. 1b shows an overview of the computational platform for the design of de novo assembled chimeric receptors. FIG. 1c shows schematics and structural models of three different selected chimera: VMR_{SHORT}, VMR_{INT}, and VMR_{FL}. FIG. 1d shows mechanical coupling between residues in the TM region (X-axis) and residues in the other domains (Y-axis) of the chimeric receptor. The mechanical couplings were derived from the correlated motions between each residue pair and calculated using Elastic Network Models and Normal Mode analysis (as described in Example 1). The correlated motions between residues belonging to the TM domain are highlighted in the “TM-TM” region. FIG. 1e shows orthogonal STAT5 phosphorylation in response to 25 ng/ml rhVEGF₁₆₅ in human T cells engineered to express VMR_{SHORT}, VMR_{INT}, or VMR_{FL} or non-transduced (NT). Top panel: representative donor FACS histograms, middle panel: % pSTAT5+ cells, lower panel: peak mean fluorescence intensity (MFI) of pSTAT5. n=5 donors, mean±SD, unpaired t-test, representative of 2 independent experiments. Positive control IL15 at 10 ng/ml. Significance levels: *p<0.05, **p<0.01, ***p<0.001, ****p<0.0001. ns: not significant.

FIGS. 2a, 2b, 2c, 2d, 2e, 2f, 2g, 2h, and 2i are a set of diagrams showing that VMR_{FL} activation selectively enhances CAR T cell expansion in response to human VEGF. FIG. 2a shows a schematic representation of the retroviral vector constructs. 4H5: single-chain variable fragment targeting ephrin Type-A receptor 2(EphA2). Δ: non-signaling control CAR, 28ζ: second generation CAR with CD28 and CD3ζ endo-domains, BBζ: second generation CAR with 41BB and CD3ζ endo-domains. FIG. 2b shows co-transduction efficiencies of activated T cells by FACS for each construct or combinations thereof. CD19 staining: marker of CAR transduction, VEGFR2 staining: direct detection of VMR_{FL}, n=8 donors (n=6 for A and Δ+VMR_{FL} conditions), mean±SD. FIG. 2c shows evaluation of EphA2 expression on A549 lung cancer cells and negative control isotype or U266 multiple myeloma cells by FACS. FIG. 2d shows a schematic of the sequential co-culture assay with repetitive tumor challenge. FIG. 2e shows a schematic of the co-culture conditions and expected results (top). Repetitive tumor cell killing (middle) and T cell expansion (bottom) in the sequential co-culture assay±exogenous addition of rhVEGF₁₆₅ at 25 ng/ml. n=7 (n=5 for Δ and Δ+VMR_{FL} conditions), mean±SD. FIG. 2f shows an area under the curve analysis of fold T cell expansion shown in panel 2e, from day 0 to day 20, mean±SEM, unpaired t-test. FIG. 2g shows a schematic of culture conditions and expected results (top). FIG. 2g shows the gene expression analysis of T cells re-isolated from co-cultures after tumor cell killing. A heat map of differentially expressed genes is shown. FIG. 2h shows T cell survival over time in media alone, IL2 50 U/ml or rhVEGF₁₆₅ 25 ng/ml, and no tumor challenge. n=4, mean±SD. FIG. 2i shows an area under the curve analysis of the T cell counts shown in FIG. 2g, from day 0 to day 25, mean±SEM, unpaired t-test with Welch's correction. (FIGS. 2f and 2i) Significance levels: *p<0.05, **p<0.01, ***p<0.001, ****p<0.0001. ns: not significant.

FIGS. 3a, 3b, 3c, 3d, 3e, and 3f are a set of diagrams showing that productive VMR_{FL} signaling occurs at clinically relevant VEGF levels. FIG. 3a shows purity of VMR_{FL}-engineered human activated T cells. FIG. 3b shows VEGF dose-dependent phosphorylation of STAT5 in VMR_{FL}+ T cells. FACS histograms of individual representative donors. FIG. 3c shows non-linear curve fit and calculation of EC50 (dotted line) for % pSTAT5+ cells (left) and pSTAT5 MFI(right). n=5, mean±SD. FIG. 3d shows human VEGF levels produced by A549 VEGF-KO, VEGF-WT, and VEGF-OE (overexpression, OE) cells in vitro. Average of technical duplicates. Black dotted lines indicate the linear assay range, grey dotted line indicates EC50, ++: assay saturated. FIG. 3e shows a schematic of a mouse model to determine hVEGF levels in mice injected intravenously (i.v.) with A549 VEGF-KO, VEGF-WT, or VEGF-OE cells. FIG. 3f shows hVEGF levels in lung tumor tissue lysates (left) and peripheral blood serum (right) of NSG mice 3 days after tumor cell injection, n=4-5 mice/group, mean±SD, unpaired t-test with Welch's correction. Significance levels: **p<0.01, ****p<0.0001.

FIGS. 4a, 4b, 4c, 4d, 4e, 4f, 4g, and 4h are a set of diagrams showing that VMR_{FL} activation enhances in vivo anti-tumor function of 41BBζ CAR T cells in a VEGF-dependent manner. FIG. 4a shows a schematic of the VEGF_{low} mouse model. FIG. 4b shows a summary of bioluminescent imaging (BLI) total flux [p/s], lines representing individual mice, n=5/group; representative of 2 independent experiments. FIG. 4c shows an area under the curve analysis of total flux shown in FIG. 4b, from day −7 to day 24, mean±SEM, unpaired t-test. FIG. 4d shows a schematic of the VEGF_{high} mouse model. FIGS. 4e and 4f show the BLI results (FIG. 4e shows individual mouse images, greyscale ranges from 1×10⁵ to 1×10⁶ p/sec/cm²/sr; and FIG. 4f shows a summary of total flux, lines representing individual mice). n=10 mice/group, pooled results of 2 independent experiments. FIG. 4g shows human VEGF levels in serum of surviving mice. Black dotted line: detection threshold, linear range. Unpaired t-test with Welch's correction. FIG. 4h shows survival of mice. n=10 mice/group, log-rank test. FIGS. 4c, 4g, and 4h show significance levels: *p<0.05, **p<0.01, ***p<0.001, ****p<0.0001. ns: not significant.

FIG. 5 shows stepwise assembly modeling of the VMR_{FL} chimera. The regions (linker or domains) modeled during each step are indicated. Domains 1 to 7 correspond to the extracellular domains of VEGFR2. TM and CP correspond to the transmembrane and cytoplasmic regions of the c-MPL receptor, respectively.

FIG. 6 shows a domain composition of the different VMR chimeras. Domains 1 to 7 correspond to the extracellular domains of VEGFR2. TM and CP correspond to the transmembrane (TM) and cytoplasmic (CP) regions of the c-MPL receptor, respectively.

FIGS. 7a, 7b, and 7c are a set of diagrams showing cytokine production and T cell phenotype during sequential co-cultures. FIG. 7a shows levels of cytokines and cytotoxic granules detected in supernatants of sequential co-cultures at 24 hours after tumor cell challenge. n=5, mean±SD, t-test. *p<0.05, ***p<0.001, ns: not significant. FIG. 7b shows CD4+ and CD8+ subset distribution during sequential co-cultures. n=3, mean±SD. FIG. 7c shows T cell differentiation status determined with CD45RO and CD62L expression by FACS; n=3, mean±SD.

FIGS. 8a, 8b, 8c, and 8d are a set of diagrams showing in vivo CAR T cell dose titration in the metastatic lung cancer xenograft model with A549 VEGF-WT cells (VEGF_{low} model). FIG. 8a shows a schematic of the mouse model. The goal was to identify a suboptimal CAR T cell dose that produces partial anti-tumor responses. FIG. 8b shows retroviral vectors and CAR constructs used. An IRES sequence was inserted upstream of the CAR cassettes to reduce CAR cell surface expression and attenuate CAR T cell potency. FIG. 8c shows a summary of in vivo bioluminescent imaging (BLI) over time. n=3-4 mice per group, mean±SD. FIG. 8d shows individual mouse pictures of BLI. Grey scale 4×10⁵ to 5×10⁶ p/s/cm²/sr. T cell dose of 1×10⁵ IRES-CAR T cells was retained for further experiments.

FIGS. 9a, 9b, 9c, 9d, 9e, 9f, and 9g are a set of diagrams showing a design of CMR and assessment of signaling capacity. FIG. 9a shows a schematic overview of CMR functionality. In the absence of cytokines, T cells undergo apoptosis while CMR baseline activity of CMR+ T cells promotes cell homeostasis and survival. In the presence of CSF1 from stromal cells (SC) of the TME, T cells do not respond and undergo apoptosis while CMR+ T cells proliferate as a result of full CMR activation. FIG. 9b shows schematics and structural models of CMR_{FL}, CMR_{INT} and CMR_{SHORT}. FIG. 9c shows mechanical coupling between residues in the TM region (X-axis) and residues in the other domains (Y-axis) of the chimeric receptor. The mechanical couplings are derived from the correlated motions between each residue pair and calculated using Elastic Network Models and Normal Mode analysis. The correlated motions between residues that both belong to the TM domain are highlighted in the “TM-TM” region. FIG. 9d shows STAT5 phosphorylation in response to 10 ng/ml rhCSF1 in human T cells engineered to express CMR_{FL}, CMR_{INT} or CMR_{SHORT} or non-transduced (NT), representative donor FACS histograms. FIG. 9e shows mean fluorescence intensity (MFI) of pSTAT5. N=3 donors, mean±SD, unpaired t-test, *p<0.05, **p<0.01, ns: not significant. FIG. 9f shows dose-dependent phosphorylation of STAT5 in CMR+ T cells upon stimulation with rhCSF1 for 30 minutes including IL-15 and NT media condition as positive and negative controls, respectively. FACS histograms of a representative individual donor. FIG. 9g shows quantification of phosphorylated STAT5 at increasing concentrations of rhCSF1 in CMR+ T cells with NT in media only or IL-15 control (10 ng/ml) after 30 minutes incubation, n=3 independent donors, mean±SD and individual values, Welch's t-test. * P<0.05, ** P<0.01, *** P<0.001.

FIGS. 10a, 10b, and 10c are a set of diagrams showing generation and In vitro characterization of CMR+ T cells. FIG. 10a shows a schematic of CMR plasmid with endogenous CSF1R signal peptide and IgG signal peptide. FIG. 10b shows transduction efficiency of CMR_{CRSP} and CMR_{IGSP} determined by flow cytometry for CSF1R, n=4 independent donors, mean±SD and individual values. NT, non-transduced. FIG. 10c shows quantitated in vitro persistence and expansion of CMR+ or NT T cells in media only (left), CSF1 (25 ng/ml) (center) or IL-2(50 U/ml) (right), n=5 independent donors, mean±SD, Welch's t-test on AUC. * P<0.05, ** P<0.01, *** P<0.001; **** P<0.0001.

FIGS. 11a, 11b, 11c, 11d, and 11e are a set of diagrams showing that CAR T cell sequential killing and expansion is enhanced by coexpression of CMR. FIG. 11a shows a schematic of CMR, CAR, and CAR+CMR plasmids. The CAR simultaneously targets B cell maturation antigen (BCMA) and transmembrane interactor and CAML interactor (TACI) expressed on multiple myeloma. The CAR is using a truncated sequence of A proliferation ligand (APRIL) in a monomeric configuration (m). FIG. 11b shows transduction efficiencies of engineered T cells, n=6 independent donors, except for m28ζCMR (n=3). Mean±SD. CMR (43.8±10.24%), m4 (82.27±7.17%), m28((68.37±12.03%), mBBζ (71.85±8.03%), mζ.CCMR (43.75±10.23%), m28ζ.CMR (29.83±12.60%) and mBBζ.CMR (47.05±8.85%). FIG. 11c shows the results of a serial killing assay of CAR T cells challenged with multiple myeloma cell lines NCI-H929 (BCMA++TACI−) or MM.1S (BCMA+TACI+) at an E:T ratio of 1:10, n=6 independent donors for all conditions except for mζ.CMR and m28ζ.CMR challenged with MM.1S (n=3), Kaplan-Meier analysis with log-rank (Mantel-Cox) test, * P<0.05, ** P<0.01. FIG. 11d shows T cell expansion after sequential tumor challenge with NCI-H929 or MM.1S, n=6 independent donors for all conditions except for mζ.CMR and m2ζ.CMR challenged with MM.1S (n=3), mean±SD, Welch's t-test on AUC from challenge 0 until 4. * P<0.05; ** P<0.01; *** P<0.001; **** P<0.0001. FIG. 11e shows cytokine production of CAR+, CMR+, and CAR+CMR T cells after 24 h culture without tumor or with NCI-H929 or MM.1S.

FIGS. 12a, 12b, 12c, 12d, 12e, 12f, 12g, and 12h are a set of diagrams showing that CMR coexpression enhances in vivo anti-tumor function of first generation (ζ) CAR T cells in a CSF-1 dependent manner. FIG. 12a shows a scheme of experimental setup/timeline for mouse xenograft model with NCI-H929.GFP.ffluc and ACT, a model where no human CSF-1 is produced by the tumor, BLI, bioluminescent imaging; iv, intravenous. This model evaluates if the constitutive baseline signaling activity of the CMR can enhance the anti-tumor function of first generation CAR T cells. FIG. 12b shows BLI results of mice untreated or treated with mζ or mζ.CMR T cells. Grey scale 1×10⁵ to 1×10⁶ p/sec/cm². FIG. 12c shows a summary of total flux, mean±SD, multiple ANOVA test. ns, not significant. FIG. 12d shows a Kaplan-Meier survival analysis with log-rank (Mantel-Cox) test. ns, not significant. FIG. 12e shows a scheme of experimental setup/timeline for mouse xenograft model with NCI-H929_{CSF1}.GFP.ffluc and ACT, a model where human CSF-1 is produced by the tumor. This model evaluates the if the ligand-dependent activity of CMR can enhance the anti-tumor function of first generation CAR T cells. FIG. 12f shows BLI results of mice untreated or treated with mΔ, CMR, mζ or mζ.CMR T cells. Grey scale 1×10⁵ to 1×10⁶ p/sec/cm². FIG. 12g shows a summary of total flux, mean±SD, multiple ANOVA test. * P<0.05; ** P<0.01; *** P<0.001; ns, not significant. FIG. 12h shows Kaplan-Meier survival analysis with log-rank (Mantel-Cox) test. * P<0.05.

FIGS. 13a, 13b, 13c, 13d, 13e, 13f, and 13g are a set of diagrams showing that CMR coexpression enhances in vivo anti-tumor function of second generation (BB) CAR T cells in a CSF-1 dependent manner. FIG. 13a shows a scheme of experimental setup/timeline for mouse xenograft model with MM.1S.GFP.ffluc.B2MKO and ACT, a model where no human CSF-1 is produced by the tumor. This model evaluates if the constitutive baseline signaling activity of the CMR can enhance the anti-tumor function of second generation CAR T cells. FIG. 13b shows BLI results of mice untreated or treated with NT, mBBζ or mBBζ.CMR T cells. Grey scale 1×10⁴ to 1×10⁶ p/sec/cm². FIG. 13c shows a summary of total flux, mean±SD, multiple ANOVA. ns, not significant. FIG. 13d shows a scheme of experimental setup/timeline for mouse xenograft model with MM.1S_{CSF1}.GFP.ffluc B2MKO and ACT, a model where human CSF-1 is produced by the tumor. This model evaluates the if the ligand-dependent activity of CMR can enhance the anti-tumor function of second generation CAR T cells. Two different CARs are evaluated, the monomeric APRIL based CAR as above, and the human heavy chain only based CAR FHVH33 (Einstein, A., et al., Phys. Rev. 47, 777-780 (1935)). FIG. 13e shows BLI results of mice untreated or treated with NT, mBBζ, mBBζ.CMR, FHVH33.BB or FHVH33.BBζ.CMR T cells. Grey scale 1×10⁴ to 1×10⁶ p/sec/cm². FIG. 13f shows a summary of total flux, mean±SD, multiple ANOVA test. * P<0.05; *** P<0.001; **** P<0.0001; ns, not significant. FIG. 13g shows a summary of total flux, mean±SD, multiple ANOVA test. * P<0.05; **** P<0.0001; ns, not significant.

FIGS. 14a, 14b, and 14c show that transgenic TCR T cell sequential killing and expansion is enhanced by coexpression of VMR or CMR. FIG. 14a shows the results of a serial killing assay of transgenic survivin-specific TCR T cells challenged with leukemia cell line BV173 at an E:T ratio of 1:5, n=4 independent donors, Kaplan-Meier analysis with log-rank (Mantel-Cox) test, ** P<0.01; ns, not significant. FIG. 14b shows T cell expansion after sequential tumor challenge with BV173, n=4 independent donors, mean±SD. FIG. 14c shows area under the curve analysis of fold T cell expansion, mean±SEM, Welch's t-test. **** P<0.0001; ns, not significant.

### DETAILED DESCRIPTION OF THE INVENTION

This disclosure presents a method for de novo bottom-up assembly and rational design of allosteric biosensors (e.g., chimeric receptors) with programmable input-output behaviors that respond to soluble factors selectively enriched in tumors and trigger co-stimulation and cytokine signals. Notably, the biosensors as designed exhibit constitutive signaling activities or are completely ligand inducible.

This disclosure demonstrates that co-expression of a chimeric antigen receptor and a biosensor in human T cells selectively mediated enhanced anti-tumor responses in metastatic lung cancer and in multiple myeloma, tumors that are rich in the targeted soluble factor. The improved anti-tumor function was associated with an enhanced immune-stimulatory and reduced exhaustion gene signature in T cells in the lung cancer model, and dual targeting of tumor antigen and soluble factor increased therapeutic specificity.

Further, the rational design approach predicted effective mechanical coupling and biosensor signaling potency correlated with anti-tumor function and represents a powerful strategy to overcome major hurdles in T cell immunotherapy for both solid tumors and hematologic malignancies. This disclosure provides for the accelerated development of synthetic biosensors with custom-built sensing and responses for basic and translational cell engineering applications.

### Allosteric Biosensors

a. Chimeric Polypeptides

In one aspect, this disclosure provides a chimeric polypeptide, comprising: (i) an extracellular ligand-binding domain capable of binding to an input signal associated with cancer, (ii) an intracellular signaling domain capable of activating and/or enhancing an anti-tumor function, and (iii) a transmembrane domain linking the extracellular ligand-binding domain and the intracellular signaling domain, wherein the extracellular ligand-binding domain and the intracellular signaling domain are derived from different receptors, and wherein binding of the input signal to the extracellular ligand-binding domain induces oligomerization of the chimeric polypeptide, and activates and/or enhances the anti-tumor function.

In some embodiments, the input signal is present in a tumor microenvironment. In some embodiments, the cancer comprises a solid tumor. In some embodiments, the cancer comprises a hematologic malignancy.

In some embodiments, the input signal comprises a growth factor, cytokine, or interleukin. In some embodiments, the input signal comprises vascular endothelial growth factor (VEGF), basic fibroblast growth factor (bFGF), platelet-derived growth factor (PDGF), hepatocyte growth factor (HGF), insulin-like growth factor (IGF), interleukin-8 (IL-8), TGFβ, IL-10, or colony stimulating factor 1 (CSF-1), interleukin-34 (IL-34), stem cell factor (SCF), interleukin-9 (IL-9), interleukin-4 (IL-4), interleukin-6 (IL-6), interleukin-10 (IL-10), Angiopoietin-1 (Ang1) (receptor is Tie2, or CD202), Thrombopoietin (TPO), Osteopontin (OPN), Receptor activator of nuclear factor kappa beta (NFkB) ligand, RANK ligand (RANKL), Fibroblast growth factor (FGF-1, -2), Vascular cell adhesion protein 1 (VCAM-1), Notch ligands: Jagged1, Jagged2, Delta-like1, Delta-like3, Delta-like4, GM-SCF/CSF2, G-CSF/CSF3, IL-1b, MMPs (such as MMP-1, MMP-2, MMP-3, MMP-7, MMP-8, MMP-9, MMP-13, MMP-14, TIMP-1, TIMP-2, TIMP-3, or TIMP-4), prostaglandin ligands (such as PGD2, PGE2, PGF2α, PGI2, TXA2, or PGH2), BMPs (such as: BMP1, BMP2, BMP3, BMP4, BMP5, BMP6, BMP7, BMP8, BMP9, BMP10, BMP11, BMP12, BMP13, BMP14, or BMP15), or adenosine receptors agonists (such as N6-Cyclopentyladenosine, N6-3-methoxyl-4-hydroxybenzyl adenine riboside (B2), adenosine, CCPA, Certain Benzodiazepines and Barbiturates, 2′-MeCCPA, GR 79236, SDZ WAG 994, Benzyloxy-cyclopentyladenosine (BnOCPA), N6-3-methoxyl-4-hydroxybenzyl adenine riboside (B2), ATL-146e, CGS-21680, Regadenoson, adenosine, 5′-N-ethylcarboxamidoadenosine, BAY 60-6583, adenosine, LUF-5835, LUF-5845, 2-(1-Hexynyl)-N-methyladenosine, CF-101 (IB-MECA), Adenosine, 2-CI-IB-MECA, CP-532,903, or MRS-3558).

In some embodiments, the input signal comprises vascular endothelial growth factor (VEGF). In some embodiments, the input signal comprises CSF-1.

In some embodiments, the anti-tumor function comprises: (i) secretion of one or more cytokines from an immune cell, (ii) co-stimulation of the immune cell, (iii) cell survival of the immune cell, (iv) proliferation of the immune cell, (v) migration of the immune cell, (vi) modified metabolism of the immune cell, (vii) altered differentiation status of the immune cell, (viii) functionality of the immune cell, or a combination thereof.

As used herein, the term “oligomerization” refers to dimerization, trimerization, and tetramerization reactions as well as polymerization of molecules, such as polypeptide chains. Oligomerization can occur between the same or different molecules. In some embodiments, binding of the input signal to the extracellular ligand-binding domain induces dimerization, trimerization, tetramerization, or polymerization of the chimeric polypeptide.

As used herein, the term “chimeric protein” or “chimeric polypeptide” refers to a recombinant fusion protein, e.g., a single polypeptide having the extracellular domains described herein and, optionally, a linker. For example, in some embodiments, the chimeric protein is translated as a single peptide chain in a cell. In some embodiments, a chimeric protein refers to a recombinant protein of multiple polypeptides, e.g., multiple domains described herein, that are linked to yield a single unit, e.g., in vitro (e.g., with one or more synthetic linkers described herein).

As used herein, the term “extracellular” refers to the protein portion extended from cell surface. “Extracellular domain” or “extracellular ligand-binding domain,” as used herein, refers broadly to the portion of a protein that extends from the surface of a cell. In some embodiments, an extracellular domain refers to a portion of a transmembrane protein that is capable of interacting with the extracellular environment. In some embodiments, an extracellular domain refers to a portion of a transmembrane protein that is sufficient to bind to a ligand or receptor and effectively transmit a signal to a cell. In some embodiments, an extracellular domain is the entire amino acid sequence of a transmembrane protein which is external of a cell or the cell membrane. In some embodiments, an extracellular domain is the portion of an amino acid sequence of a transmembrane protein that is external of a cell or the cell membrane and is needed for signal transduction and/or ligand binding as may be assayed using methods known in the art (e.g., in vim ligand binding and/or cellular activation assays).

“Transmembrane domain,” as used herein, refers broadly to an amino acid sequence (e.g., with about 15 to 50 amino acid residues in length) which spans the plasma membrane. In some embodiments, a transmembrane domain includes about at least 20, 25, 30, 35, 40, or 45 amino acid residues and spans the plasma membrane. Transmembrane domains are rich in hydrophobic residues, and typically have an alpha-helical structure. In an embodiment, at least 50%, 60%, 70%, 80%, 90%, 95%, or more of the amino acids of a transmembrane domain are hydrophobic, e.g., leucines, isoleucines, tyrosines, or tryptophans. Transmembrane domains are described in, for example, Zagotta et al. (1996) Annu. Rev. Neurosci. 19:235-263.

As used herein, the term “intracellular domain,” “intracellular signaling domain,” or “cytoplasmic domain” refers to the intracellular portion of a molecule. In some embodiments, an intracellular domain transmits a signal to an effector function and causes a cell to perform a specific function, e.g., activation, phosphorylation, cytokine production, etc. The term intracellular domain is meant to include any truncated portion of the intracellular domain sufficient to transduce a signal that may be an effector, a memory, a differentiation, a degranulation, a metabolic, a proliferation, a homeostatic, a growth, or a survival signal, etc.

In some embodiments, the extracellular ligand-binding domain comprises one or more extracellular domains of VEGFR2, VEGFR1, FGFR1, PDGFR, HGFR, IGFR, IL-8R (IL-8Ra/IL-8Rb), TGFβR1, IL-10R, CSF1R, SCFR/KIT/cKIT/CD117, IL-9R, IL-4R, IL4R, IL-10R, Tie2/CD202, C-MPL/TPOR, CD44, RANK, FGFR1, FGFR2, FGFR3, and FGFR4, VLA-4, NOTCH1, NOTCH2, NOTCH3, and NOTCH4, GM-SCFR/CSF2R, G-CSFR/CSF3R, IL-1R, immune checkpoints (such as PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160), prostaglandin receptors (such as EP1, EP2, EP3, EP4, IP, TP, DP1, DP2, FP, adenosine receptors (such as A1, A2A, A2B, and A3), BMB receptors (such as BMPR1A, BMPR1B, BMPR2, ActR-IA, ActR-2A, ActR-2B), or a variant or fragment thereof.

In some embodiments, the extracellular ligand-binding domain comprises one or more extracellular domains of VEGFR2 or variants thereof. In some embodiments, the extracellular ligand-binding domain comprises one or more extracellular domains of a CSF-1 receptor (CSF-1R) or variants thereof or specific linker sequences. In some embodiments, the extracellular ligand-binding domain comprises D1-7; D1-4 and D17; or D1-3 extracellular domains of the VEGFR2.

In some embodiments, the extracellular ligand-binding domain comprises an amino acid sequence having at least 80% (e.g., 80%, 85%, 90%, 91%, 92%, 93%, 94%, 95%, 96%, 97%, 98%, 99%) sequence identity to SEQ ID NO: 3, 8, 10, 15, 17, or 19 or comprises the amino acid sequence of SEQ ID NO: 3, 8, 10, 15, 17, or 19 (see Table 1 below).

TABLE 1
Representative Sequences

| SEQ | | OTHER |
|---|---|---|
| ID NO | SEQUENCES | INFORMATION |
| 1 | MEFGLSWLFLVAILKGVQC | Signal peptide (derived |
| | | from an |
| | | immunoglobulin IgG1) |
| 2 | EQKLISEEDL | Myc tag |
| 3 | ASVGLPSVSLDLPRLSIQKDILTIKANTTLQITCRGQ | VEGFR2 extracellular |
| | RDLDWLWPNNQSGSEQRVEVTECSDGLFCKTLTIP | domain VMR_{FL} |
| | KVIGNDTGAYKCFYRETDLASVIYVYVQDYRSPFIA | Uniport P35968, aa 20- |
| | SVSDQHGVVYITENKNKTVVIPCLGSISNLNVSLCA | 964 |
| | RYPEKRFVPDGNRISWDSKKGFTIPSYMISYAGMVF | |
| | CEAKINDESYQSIMYIVVVVGYRIYDVVLSPSHGIEL | |
| | SVGEKLVLNCTARTELNVGIDFNWEYPSSKHQHKK | |
| | LVNRDLKTQSGSEMKKFLSTLTIDGVTRSDQGLYT | |
| | CAASSGLMTKKNSTFVRVHEKPFVAFGSGMESLVE | |
| | ATVGERVRIPAKYLGYPPPEIKWYKNGIPLESNHTI | |
| | KAGHVLTIMEVSERDTGNYTVILTNPISKEKQSHVV | |
| | SLVVYVPPQIGEKSLISPVDSYQYGTTQTLTCTVYAI | |
| | PPPHHIHWYWQLEEECANEPSQAVSVTNPYPCEEW | |
| | RSVEDFQGGNKIEVNKNQFALIEGKNKTVSTLVIQA | |
| | ANVSALYKCEAVNKVGRGERVISFHVTRGPEITLQP | |
| | DMQPTEQESVSLWCTADRSTFENLTWYKLGPQPLP | |
| | IHVGELPTPVCKNLDTLWKLNATMFSNSTNDILIME | |
| | LKNASLQDQGDYVCLAQDRKTKKRHCVVRQLTVL | |
| | ERVAPTITGNLENQTTSIGESIEVSCTASGNPPPQIM | |
| | WFKDNETLVEDSGIVLKDGNRNLTIRRVRKEDEGL | |
| | YTCQACSVLGCAKVEAFFIIEGAQEKTNLE | |
| 4 | RVETATETAW | c-MPL hinge |
| | | Uniprot P40238-1, aa |
| | | 482-491 |
| 5 | ISLVTALHLVLGLSAVLGLLLL | c-MPL transmembrane |
| | | Uniprot P40238-1, aa |
| | | 492-513 |
| 6 | RWQFPAHYRRLRHALWPSLPDLHRVLGQYLRDTA | c-MPL juxtamembrane |
| | ALSPPKATVSDTCEEVEPSLLEILPKSSERTPLPLCSS | and cytoplasmic domain |
| | QAQMDYRRLQPSCLGTMPLSVCPPMAESGSCCTTH | Uniprot P40238-1, aa |
| | IANHSYLPLSYWQQP | 514-635 |
| 7 | MEFGLSWLFLVAILKGVQCEQKLISEEDLASVGLPSV | VMR_{FL} Construct |
| | SLDLPRLSIQKDILTIKANTTLQITCRGQRDLDWLWPN | Italicized and |
| | NQSGSEQRVEVTECSDGLFCKTLTIPKVIGNDTGAYKC | underlined: Signal |
| | FYRETDLASVIYVYVQDYRSPFIASVSDQHGVVYITENK | peptide |
| | NKTVVIPCLGSISNLNVSLCARYPEKRFVPDGNRISWDS | Bolded: Myc tag |
| | KKGFTIPSYMISYAGMVFCEAKINDESYQSIMYIVVVVG | Italicized: extracellular |
| | YRIYDVVLSPSHGIELSVGEKLVLNCTARTELNVGIDFN | domain (Uniport |
| | WEYPSSKHQHKKLVNRDLKTQSGSEMKKFLSTLTIDG | P35968, aa 20-964) |
| | VTRSDQGLYTCAASSGLMTKKNSTFVRVHEKPFVAFGS | Italicized, underlined, |
| | GMESLVEATVGERVRIPAKYLGYPPPEIKWYKNGIPLE | and bolded: c-MPL |
| | SNHTIKAGHVLTIMEVSERDTGNYTVILTNPISKEKQSH | hinge (Uniprot P40238- |
| | VVSLVVYVPPQIGEKSLISPVDSYQYGTTQTLTCTVYAIP | 1, aa 492-513) |
| | PPHHIHWYWQLEEECANEPSQAVSVTNPYPCEEWRSV | Italicized and bolded: |
| | EDFQGGNKIEVNKNQFALIEGKNKTVSTLVIQAANVSA | transmembrane domain |
| | LYKCEAVNKVGRGERVISFHVTRGPEITLQPDMQPTE | (Uniprot P40238-1, aa |
| | QESVSLWCTADRSTFENLTWYKLGPQPLPIHVGELPTP | 492-513) |
| | VCKNLDTLWKLNATMFSNSTNDILIMELKNASLQDQG | Double-underlined: |
| | DYVCLAQDRKTKKRHCVVRQLTVLERVAPTITGNLEN | juxtamembrane and |
| | QTTSIGESIEVSCTASGNPPPQIMWFKDNETLVEDSGIV | cytoplasmic domain |
| | LKDGNRNLTIRRVRKEDEGLYTCQACSVLGCAKVEAF | (Uniprot P40238-1, aa |
| | FIIEGAQEKTNLERVETATETAWISLVTALHLVLGLS | 514-635) |
| | AVLGLLLLRWQFPAHYRRLRHALWPSLPDLHRVLG | |
| | QYLRDTAALSPPKATVSDTCEEVEPSLLEILPKSSER | |
| | TPLPLCSSQAQMDYRRLQPSCLGTMPLSVCPPMAE | |
| | SGSCCTTHIANHSYLPLSYWQQP | |
| 8 | ASVGLPSVSLDLPRLSIQKDILTIKANTTLQITCRGQ | VEGFR2 extracellular |
| | RDLDWLWPNNQSGSEQRVEVTECSDGLFCKTLTIP | domain VMR_{INT} |
| | KVIGNDTGAYKCFYRETDLASVIYVYVQDYRSPFIA | Uniport P35968, aa 20- |
| | SVSDQHGVVYITENKNKTVVIPCLGSISNLNVSLCA | 420 and aa 661-764 |
| | RYPEKRFVPDGNRISWDSKKGFTIPSYMISYAGMVF | (underlined) |
| | CEAKINDESYQSIMYIVVVVGYRIYDVVLSPSHGIEL | |
| | SVGEKLVLNCTARTELNVGIDFNWEYPSSKHQHKK | |
| | LVNRDLKTQSGSEMKKFLSTLTIDGVTRSDQGLYT | |
| | CAASSGLMTKKNSTFVRVHEKPFVAFGSGMESLVE | |
| | ATVGERVRIPAKYLGYPPPEIKWYKNGIPLESNHTI | |
| | KAGHVLTIMEVSERDTGNYTVILTNPISKEKQSHVV | |
| | SLVVYVPVLERVAPTITGNLENQTTSIGESIEVSCTA | |
| | SGNPPPQIMWFKDNETLVEDSGIVLKDGNRNLTIRR | |
| | VRKEDEGLYTCQACSVLGCAKVEAFFIIEGAQEKT | |
| | NLE | |
| 9 | MEFGLSWLFLVAILKGVQCEQKLISEEDLASVGLPSV | VMR_{INT} Construct |
| | SLDLPRLSIQKDILTIKANTTLQITCRGQRDLDWLWPN | Italicized and |
| | NQSGSEQRVEVTECSDGLFCKTLTIPKVIGNDTGAYKC | underlined: Signal |
| | FYRETDLASVIYVYVQDYRSPFIASVSDQHGYVYITENK | peptide (derived from an |
| | NKTVVIPCLGSISNLNVSLCARYPEKRFVPDGNRISWDS | immunoglobulin IgG1) |
| | KKGFTIPSYMISYAGMVFCEAKINDESYQSIMYIVVVVG | Bolded: Myc tag |
| | YRIYDVVLSPSHGIELSVGEKLVLNCTARTELNVGIDFN | Italicized: extracellular |
| | WEYPSSKHQHKKLVNRDLKTQSGSEMKKFLSTLTIDG | domain (Uniport |
| | VTRSDQGLYTCAASSGLMTKKNSTFVRVHEKPFVAFGS | P35968, aa 20-420 and |
| | GMESLVEATVGERVRIPAKYLGYPPPEIKWYKNGIPLE | aa 661-764) |
| | SNHTIKAGHVLTIMEVSERDTGNYTVILTNPISKEKQSH | Italicized, underlined, |
| | VVSLVVYVPVLERVAPTITGNLENQTTSIGESIEVSCTAS | and bolded: c-MPL |
| | GNPPPQIMWFKDNETLVEDSGIVLKDGNRNLTIRRVR | hinge (Uniprot P40238- |
| | KEDEGLYTCQACSVLGCAKVEAFFIIEGAQEKTNLERV | 1, aa 482-491) |
| | ETATETAWISLVTALHLVLGLSAVLGLLLLRWQFPA | Italicized and bolded: |
| | HYRRLRHALWPSLPDLHRVLGQYLRDTAALSPPKA | transmembrane domain |
| | TVSDTCEEVEPSLLEILPKSSERTPLPLCSSQAQMDY | (Uniprot P40238-1, aa |
| | RRLQPSCLGTMPLSVCPPMAESGSCCTTHIANHSYL | 492-513) |
| | PLSYWQQP | Double-underlined: |
| | | juxtamembrane and |
| | | cytoplasmic domain |
| | | (Uniprot P40238-1, aa |
| | | 514-635) |
| 10 | ASVGLPSVSLDLPRLSIQKDILTIKANTTLQITCRGQ | VEGFR2 extracellular |
| | RDLDWLWPNNQSGSEQRVEVTECSDGLFCKTLTIP | domain VMR_{SHORT} |
| | KVIGNDTGAYKCFYRETDLASVIYVYVQDYRSPFIA | Uniport P35968, aa 20- |
| | SVSDQHGVVYITENKNKTVVIPCLGSISNLNVSLCA | 327 and 754-764 |
| | RYPEKRFVPDGNRISWDSKKGFTIPSYMISYAGMVF | |
| | CEAKINDESYQSIMYIVVVVGYRIYDVVLSPSHGIEL | |
| | SVGEKLVLNCTARTELNVGIDFNWEYPSSKHQHKK | |
| | LVNRDLKTQSGSEMKKFLSTLTIDGVTRSDQGLYT | |
| | CAASSGLMTKKNSTFVRVHEKIEGAQEKTNLE | |
| 11 | MEFGLSWLFLVAILKGVQCEQKLISEEDLASVGLPSV | VMR_{SHORT} Construct |
| | SLDLPRLSIQKDILTIKANTTLQITCRGQRDLDWLWPN | Italicized and |
| | NQSGSEQRVEVTECSDGLFCKTLTIPKVIGNDTGAYKC | underlined: Signal |
| | FYRETDLASVIYVYVQDYRSPFIASVSDQHGVVYITENK | peptide (derived from an |
| | NKTVVIPCLGSISNLNVSLCARYPEKRFVPDGNRISWDS | immunoglobulin IgG1) |
| | KKGFTIPSYMISYAGMVFCEAKINDESYQSIMYIVVVVG | Bolded: Myc tag |
| | YRIYDVVLSPSHGIELSYGEKLVLNCTARTELNVGIDFN | Italicized: extracellular |
| | WEYPSSKHQHKKLVNRDLKTQSGSEMKKFLSTLTIDG | domain (Uniport |
| | VTRSDQGLYTCAASSGLMTKKNSTFVRVHEKIEGAQEK | P35968, aa 20-327 and |
| | TNLERVETATETAWISLVTALHLVLGLSAVLGLLLL | Uniprot P35968, aa 754- |
| | RWQFPAHYRRLRHALWPSLPDLHRVLGQYLRDTA | 764) |
| | ALSPPKATVSDTCEEVEPSLLEILPKSSERTPLPLCSS | Italicized, underlined, |
| | QAQMDYRRLQPSCLGTMPLSVCPPMAESGSCCTTH | and bolded: c-MPL |
| | IANHSYLPLSYWQQP | hinge (Uniprot P40238- |
| | | 1, aa 482-491) |
| | | Italicized and bolded: |
| | | transmembrane domain |
| | | (Uniprot P40238-1, aa |
| | | 492-513) |
| | | Double-underlined: |
| | | juxtamembrane and |
| | | cytoplasmic domain |
| | | (Uniprot P40238-1, aa |
| | | 514-635) |
| 12 | ATGGAATTCGGCCTGAGCTGGCTGTTCCTGGTGG | VMR_{FL} Construct |
| | CCATCCTGAAGGGCGTGCAGTGCGAGCAGAAGC | |
| | TGATCTCCGAAGAGGACCTGGCCAGCGTGGGACT | |
| | GCCTAGCGTGTCACTGGACCTGCCCAGACTGAGC | |
| | ATCCAGAAGGACATCCTGACCATCAAGGCCAAC | |
| | ACCACCCTGCAGATCACCTGTCGGGGCCAGAGGG | |
| | ACCTGGATTGGCTGTGGCCCAACAACCAGAGCGG | |
| | CAGCGAGCAGAGGGTGGAAGTGACCGAGTGTAG | |
| | CGACGGCCTGTTCTGCAAGACCCTGACAATCCCC | |
| | AAAGTGATCGGCAACGACACCGGCGCCTACAAG | |
| | TGCTTCTACAGAGAGACAGACCTGGCCTCCGTGA | |
| | TCTACGTGTACGTGCAGGACTACAGAAGCCCCTT | |
| | TATCGCCAGCGTGTCCGACCAGCACGGCGTGGTG | |
| | TACATCACCGAGAACAAGAACAAGACCGTCGTG | |
| | ATCCCCTGCCTGGGCAGCATCAGCAACCTGAACG | |
| | TGTCCCTGTGCGCCAGATACCCCGAGAAGAGATT | |
| | CGTGCCCGACGGCAACCGGATCAGCTGGGACAG | |
| | CAAGAAGGGCTTCACCATCCCCAGCTACATGATC | |
| | AGCTACGCCGGCATGGTGTTCTGCGAGGCCAAGA | |
| | TCAACGACGAGAGCTACCAGAGCATCATGTACAT | |
| | CGTCGTGGTCGTGGGCTACCGGATCTACGACGTG | |
| | GTGCTGAGCCCTAGCCACGGCATCGAGCTGTCTG | |
| | TGGGCGAGAAGCTGGTGCTGAACTGCACCGCCA | |
| | GAACCGAGCTGAACGTGGGCATCGACTTCAACTG | |
| | GGAGTACCCCAGCAGCAAGCACCAGCACAAGAA | |
| | ACTCGTGAACCGGGACCTGAAAACCCAGTCCGGC | |
| | TCCGAGATGAAGAAGTTCCTGAGCACACTGACCA | |
| | TCGACGGCGTGACCAGAAGCGACCAGGGCCTGT | |
| | ACACATGTGCCGCCAGCAGCGGCCTGATGACCAA | |
| | GAAAAACAGCACCTTCGTGCGGGTGCACGAGAA | |
| | GCCTTTCGTGGCCTTTGGCAGCGGCATGGAAAGC | |
| | CTGGTGGAAGCCACCGTGGGAGAGAGAGTGCGG | |
| | ATCCCCGCCAAGTACCTGGGCTACCCTCCACCCG | |
| | AGATCAAGTGGTACAAGAACGGCATCCCCCTGG | |
| | AAAGCAACCACACAATCAAGGCCGGCCACGTGC | |
| | TGACAATCATGGAAGTGTCTGAGCGGGACACCG | |
| | GCAATTACACCGTGATCCTGACTAACCCCATCAG | |
| | CAAAGAAAAGCAGAGCCACGTGGTGTCCCTGGT | |
| | GGTGTATGTGCCCCCCCAGATCGGCGAGAAGTCC | |
| | CTGATCAGCCCCGTGGACAGCTACCAGTACGGCA | |
| | CCACCCAGACACTGACCTGCACCGTGTACGCCAT | |
| | CCCTCCCCCTCACCACATCCACTGGTACTGGCAG | |
| | CTGGAAGAGGAATGCGCCAACGAGCCTAGCCAG | |
| | GCCGTGTCCGTGACCAACCCCTACCCTTGTGAAG | |
| | AGTGGCGGAGCGTGGAAGATTTCCAGGGCGGCA | |
| | ACAAGATCGAAGTGAACAAGAATCAGTTCGCCCT | |
| | GATCGAGGGCAAAAACAAGACAGTGTCCACCCT | |
| | CGTGATCCAGGCCGCCAATGTGTCCGCCCTGTAC | |
| | AAGTGCGAGGCCGTGAACAAAGTGGGCAGAGGC | |
| | GAGAGAGTGATCTCCTTCCACGTGACCCGGGGAC | |
| | CAGAGATCACACTGCAGCCCGACATGCAGCCCAC | |
| | CGAGCAGGAATCCGTGTCTCTGTGGTGCACCGCC | |
| | GACCGGTCCACCTTCGAGAACCTGACCTGGTACA | |
| | AGCTGGGCCCCCAGCCCCTGCCTATCCACGTGGG | |
| | AGAACTGCCTACCCCCGTGTGCAAGAACCTGGAC | |
| | ACCCTGTGGAAGCTGAACGCCACCATGTTCAGCA | |
| | ACAGCACCAACGATATCCTGATCATGGAACTGAA | |
| | GAACGCCAGCCTGCAGGACCAGGGCGACTACGT | |
| | GTGTCTGGCCCAGGACAGAAAGACCAAAAAGCG | |
| | GCACTGCGTCGTGCGGCAGCTGACCGTGCTGGAA | |
| | AGAGTGGCCCCTACCATCACCGGCAACCTGGAAA | |
| | ACCAGACCACCTCCATCGGCGAGTCCATCGAGGT | |
| | GTCCTGTACCGCCAGCGGCAACCCCCCACCTCAG | |
| | ATCATGTGGTTTAAGGACAACGAGACACTGGTGG | |
| | AAGATAGCGGCATCGTGCTGAAGGACGGCAACA | |
| | GAAATCTGACCATTCGCAGAGTGCGCAAAGAGG | |
| | ACGAGGGACTGTACACCTGTCAGGCCTGTAGCGT | |
| | GCTGGGCTGCGCCAAAGTGGAAGCCTTCTTTATC | |
| | ATCGAGGGCGCCCAGGAAAAGACCAATCTGGAA | |
| | AGGGTGGAGACCGCCACCGAGACCGCCTGGATC | |
| | TCCTTGGTGACCGCTCTGCATCTAGTGCTGGGCCT | |
| | CAGCGCCGTCCTGGGCCTGCTGCTGCTGAGGTGG | |
| | CAGTTTCCTGCACACTACAGGAGACTGAGGCATG | |
| | CCCTGTGGCCCTCACTTCCAGACCTGCACCGGGT | |
| | CCTAGGCCAGTACCTTAGGGACACTGCAGCCCTG | |
| | AGCCCGCCCAAGGCCACAGTCTCAGATACCTGTG | |
| | AAGAAGTGGAACCCAGCCTCCTTGAAATCCTCCC | |
| | CAAGTCCTCAGAGAGGACTCCTTTGCCCCTGTGT | |
| | TCCTCCCAGGCCCAGATGGACTACCGAAGATTGC | |
| | AGCCTTCTTGCCTGGGGACCATGCCCCTGTCTGT | |
| | GTGCCCACCCATGGCTGAGTCAGGGTCCTGCTGT | |
| | ACCACCCACATTGCCAACCATTCCTACCTACCAC | |
| | TAAGCTATTGGCAGCAGCCTTAA | |
| 13 | ATGGAATTCGGCCTGAGCTGGCTGTTCCTGGTGG | VMR_{INT} Construct |
| | CCATCCTGAAGGGCGTGCAGTGCGAGCAGAAGC | |
| | TGATCTCCGAAGAGGACCTGGCCAGCGTGGGACT | |
| | GCCTAGCGTGTCACTGGACCTGCCCAGACTGAGC | |
| | ATCCAGAAGGACATCCTGACCATCAAGGCCAAC | |
| | ACCACCCTGCAGATCACCTGTCGGGGCCAGAGGG | |
| | ACCTGGATTGGCTGTGGCCCAACAACCAGAGCGG | |
| | CAGCGAGCAGAGGGTGGAAGTGACCGAGTGTAG | |
| | CGACGGCCTGTTCTGCAAGACCCTGACAATCCCC | |
| | AAAGTGATCGGCAACGACACCGGCGCCTACAAG | |
| | TGCTTCTACAGAGAGACAGACCTGGCCTCCGTGA | |
| | TCTACGTGTACGTGCAGGACTACAGAAGCCCCTT | |
| | TATCGCCAGCGTGTCCGACCAGCACGGCGTGGTG | |
| | TACATCACCGAGAACAAGAACAAGACCGTCGTG | |
| | ATCCCCTGCCTGGGCAGCATCAGCAACCTGAACG | |
| | TGTCCCTGTGCGCCAGATACCCCGAGAAGAGATT | |
| | CGTGCCCGACGGCAACCGGATCAGCTGGGACAG | |
| | CAAGAAGGGCTTCACCATCCCCAGCTACATGATC | |
| | AGCTACGCCGGCATGGTGTTCTGCGAGGCCAAGA | |
| | TCAACGACGAGAGCTACCAGAGCATCATGTACAT | |
| | CGTCGTGGTCGTGGGCTACCGGATCTACGACGTG | |
| | GTGCTGAGCCCTAGCCACGGCATCGAGCTGTCTG | |
| | TGGGCGAGAAGCTGGTGCTGAACTGCACCGCCA | |
| | GAACCGAGCTGAACGTGGGCATCGACTTCAACTG | |
| | GGAGTACCCCAGCAGCAAGCACCAGCACAAGAA | |
| | ACTCGTGAACCGGGACCTGAAAACCCAGTCCGGC | |
| | TCCGAGATGAAGAAGTTCCTGAGCACACTGACCA | |
| | TCGACGGCGTGACCAGAAGCGACCAGGGCCTGT | |
| | ACACATGTGCCGCCAGCAGCGGCCTGATGACCAA | |
| | GAAAAACAGCACCTTCGTGCGGGTGCACGAGAA | |
| | GCCTTTCGTGGCCTTTGGCAGCGGCATGGAAAGC | |
| | CTGGTGGAAGCCACCGTGGGAGAGAGAGTGCGG | |
| | ATCCCCGCCAAGTACCTGGGCTACCCTCCACCCG | |
| | AGATCAAGTGGTACAAGAACGGCATCCCCCTGG | |
| | AAAGCAACCACACAATCAAGGCCGGCCACGTGC | |
| | TGACAATCATGGAAGTGTCTGAGCGGGACACCG | |
| | GCAATTACACCGTGATCCTGACTAACCCCATCAG | |
| | CAAAGAAAAGCAGAGCCACGTGGTGTCCCTGGT | |
| | GGTGTATGTGCCCGTGCTGGAAAGAGTGGCCCCT | |
| | ACCATCACCGGCAACCTGGAAAACCAGACCACCT | |
| | CCATCGGCGAGTCCATCGAGGTGTCCTGTACCGC | |
| | CAGCGGCAACCCCCCACCTCAGATCATGTGGTTT | |
| | AAGGACAACGAGACACTGGTGGAAGATAGCGGC | |
| | ATCGTGCTGAAGGACGGCAACAGAAATCTGACC | |
| | ATTCGCAGAGTGCGCAAAGAGGACGAGGGACTG | |
| | TACACCTGTCAGGCCTGTAGCGTGCTGGGCTGCG | |
| | CCAAAGTGGAAGCCTTCTTTATCATCGAGGGCGC | |
| | CCAGGAAAAGACCAATCTGGAAAGGGTGGAGAC | |
| | CGCCACCGAGACCGCCTGGATCTCCTTGGTGACC | |
| | GCTCTGCATCTAGTGCTGGGCCTCAGCGCCGTCC | |
| | TGGGCCTGCTGCTGCTGAGGTGGCAGTTTCCTGC | |
| | ACACTACAGGAGACTGAGGCATGCCCTGTGGCCC | |
| | TCACTTCCAGACCTGCACCGGGTCCTAGGCCAGT | |
| | ACCTTAGGGACACTGCAGCCCTGAGCCCGCCCAA | |
| | GGCCACAGTCTCAGATACCTGTGAAGAAGTGGA | |
| | ACCCAGCCTCCTTGAAATCCTCCCCAAGTCCTCA | |
| | GAGAGGACTCCTTTGCCCCTGTGTTCCTCCCAGG | |
| | CCCAGATGGACTACCGAAGATTGCAGCCTTCTTG | |
| | CCTGGGGACCATGCCCCTGTCTGTGTGCCCACCC | |
| | ATGGCTGAGTCAGGGTCCTGCTGTACCACCCACA | |
| | TTGCCAACCATTCCTACCTACCACTAAGCTATTG | |
| | GCAGCAGCCTTAA | |
| 14 | ATGGAATTCGGCCTGAGCTGGCTGTTCCTGGTGG | VMR_{SHORT} Construct |
| | CCATCCTGAAGGGCGTGCAGTGCGAGCAGAAGC | |
| | TGATCTCCGAAGAGGACCTGGCCAGCGTGGGACT | |
| | GCCTAGCGTGTCACTGGACCTGCCCAGACTGAGC | |
| | ATCCAGAAGGACATCCTGACCATCAAGGCCAAC | |
| | ACCACCCTGCAGATCACCTGTCGGGGCCAGAGGG | |
| | ACCTGGATTGGCTGTGGCCCAACAACCAGAGCGG | |
| | CAGCGAGCAGAGGGTGGAAGTGACCGAGTGTAG | |
| | CGACGGCCTGTTCTGCAAGACCCTGACAATCCCC | |
| | AAAGTGATCGGCAACGACACCGGCGCCTACAAG | |
| | TGCTTCTACAGAGAGACAGACCTGGCCTCCGTGA | |
| | TCTACGTGTACGTGCAGGACTACAGAAGCCCCTT | |
| | TATCGCCAGCGTGTCCGACCAGCACGGCGTGGTG | |
| | TACATCACCGAGAACAAGAACAAGACCGTCGTG | |
| | ATCCCCTGCCTGGGCAGCATCAGCAACCTGAACG | |
| | TGTCCCTGTGCGCCAGATACCCCGAGAAGAGATT | |
| | CGTGCCCGACGGCAACCGGATCAGCTGGGACAG | |
| | CAAGAAGGGCTTCACCATCCCCAGCTACATGATC | |
| | AGCTACGCCGGCATGGTGTTCTGCGAGGCCAAGA | |
| | TCAACGACGAGAGCTACCAGAGCATCATGTACAT | |
| | CGTCGTGGTCGTGGGCTACCGGATCTACGACGTG | |
| | GTGCTGAGCCCTAGCCACGGCATCGAGCTGTCTG | |
| | TGGGCGAGAAGCTGGTGCTGAACTGCACCGCCA | |
| | GAACCGAGCTGAACGTGGGCATCGACTTCAACTG | |
| | GGAGTACCCCAGCAGCAAGCACCAGCACAAGAA | |
| | ACTCGTGAACCGGGACCTGAAAACCCAGTCCGGC | |
| | TCCGAGATGAAGAAGTTCCTGAGCACACTGACCA | |
| | TCGACGGCGTGACCAGAAGCGACCAGGGCCTGT | |
| | ACACATGTGCCGCCAGCAGCGGCCTGATGACCAA | |
| | GAAAAACAGCACCTTCGTGCGGGTGCACGAGAA | |
| | GATCGAGGGCGCCCAGGAAAAGACCAATCTGGA | |
| | AAGGGTGGAGACCGCCACCGAGACCGCCTGGAT | |
| | CTCCTTGGTGACCGCTCTGCATCTAGTGCTGGGC | |
| | CTCAGCGCCGTCCTGGGCCTGCTGCTGCTGAGGT | |
| | GGCAGTTTCCTGCACACTACAGGAGACTGAGGCA | |
| | TGCCCTGTGGCCCTCACTTCCAGACCTGCACCGG | |
| | GTCCTAGGCCAGTACCTTAGGGACACTGCAGCCC | |
| | TGAGCCCGCCCAAGGCCACAGTCTCAGATACCTG | |
| | TGAAGAAGTGGAACCCAGCCTCCTTGAAATCCTC | |
| | CCCAAGTCCTCAGAGAGGACTCCTTTGCCCCTGT | |
| | GTTCCTCCCAGGCCCAGATGGACTACCGAAGATT | |
| | GCAGCCTTCTTGCCTGGGGACCATGCCCCTGTCT | |
| | GTGTGCCCACCCATGGCTGAGTCAGGGTCCTGCT | |
| | GTACCACCCACATTGCCAACCATTCCTACCTACC | |
| | ACTAAGCTATTGGCAGCAGCCTTAA | |
| 1 | MEFGLSWLFLVAILKGVQC | CMR_{FL} |
| | | Signal peptide (derived |
| | | from an |
| | | immunoglobulin IgG1) |
| 15 | IPVIEPSVPELVVKPGATVTLRCVGNGSVEWDGPPS | CMR_{FL} |
| | PHWTLYSDGSSSILSTNNATFQNTGTYRCTEPGDPL | CSF1R extracellular |
| | GGSAAIHLYVKDPARPWNVLAQEVVVFEDQDALL | domain (Uniprot |
| | PCLLTDPVLEAGVSLVRVRGRPLMRHTNYSFSPWH | P07333, aa 20-517) |
| | GFTIHRAKFIQSQDYQCSALMGGRKVMSISIRLKVQ | |
| | KVIPGPPALTLVPAELVRIRGEAAQIVCSASSVDVNF | |
| | DVFLQHNNTKLAIPQQSDFHNNRYQKVLTLNLDQV | |
| | DFQHAGNYSCVASNVQGKHSTSMFFRVVESAYLN | |
| | LSSEQNLIQEVTVGEGLNLKVMVEAYPGLQGFNWT | |
| | YLGPFSDHQPEPKLANATTKDTYRHTFTLSLPRLKP | |
| | SEAGRYSFLARNPGGWRALTFELTLRYPPEVSVIWT | |
| | FINGSGTLLCAASGYPQPNVTWLQCSGHTDRCDEA | |
| | QVLQVWDDPYPEVLSQEPFHKVTVQSLLTVETLEH | |
| | NQTYECRAHNSVGSGSWAFIPISAGAHTHPPDEFLF | |
| | TP | |
| 4 | RVETATETAW | CMR_{FL} |
| | | c-MPL hinge (Uniprot |
| | | P40238-1, aa 482-491) |
| 5 | ISLVTALHLVLGLSAVLGLLLL | CMR_{FL} |
| | | c-MPL transmembrane |
| | | (Uniprot P40238-1, aa |
| | | 492-513) |
| 6 | RWQFPAHYRRLRHALWPSLPDLHRVLGQYLRDTA | CMR_{FL} |
| | ALSPPKATVSDTCEEVEPSLLEILPKSSERTPLPLCSS | c-MPL juxtamembrane |
| | QAQMDYRRLQPSCLGTMPLSVCPPMAESGSCCTTH | and cytoplasmic domain |
| | IANHSYLPLSYWQQP | (Uniprot P40238-1, aa |
| | | 514-635) |
| 16 | MEFGLSWLFLVAILKGVQCIPVIEPSVPELVVKPGA | CMR_{FL} |
| | TVTLRCVGNGSVEWDGPPSPHWTLYSDGSSSILSTN | (full construct) |
| | NATFQNTGTYRCTEPGDPLGGSAAIHLYVKDPARP | |
| | WNVLAQEVVVFEDQDALLPCLLTDPVLEAGVSLV | |
| | RVRGRPLMRHTNYSFSPWHGFTIHRAKFIQSQDYQ | |
| | CSALMGGRKVMSISIRLKVQKVIPGPPALTLVPAEL | |
| | VRIRGEAAQIVCSASSVDVNFDVFLQHNNTKLAIPQ | |
| | QSDFHNNRYQKVLTLNLDQVDFQHAGNYSCVASN | |
| | VQGKHSTSMFFRVVESAYLNLSSEQNLIQEVTVGE | |
| | GLNLKVMVEAYPGLQGFNWTYLGPFSDHQPEPKL | |
| | ANATTKDTYRHTFTLSLPRLKPSEAGRYSFLARNPG | |
| | GWRALTFELTLRYPPEVSVIWTFINGSGTLLCAASG | |
| | YPQPNVTWLQCSGHTDRCDEAQVLQVWDDPYPEV | |
| | LSQEPFHKVTVQSLLTVETLEHNQTYECRAHNSVG | |
| | SGSWAFIPISAGAHTHPPDEFLFTPRVETATETAWIS | |
| | LVTALHLVLGLSAVLGLLLLRWQFPAHYRRLRHAL | |
| | WPSLPDLHRVLGQYLRDTAALSPPKATVSDTCEEV | |
| | EPSLLEILPKSSERTPLPLCSSQAQMDYRRLQPSCLG | |
| | TMPLSVCPPMAESGSCCTTHIANHSYLPLSYWQQP | |
| 1 | MEFGLSWLFLVAILKGVQC | CMR_{INT} |
| | | Signal peptide (derived |
| | | from an |
| | | immunoglobulin IgG1) |
| 17 | IPVIEPSVPELVVKPGATVTLRCVGNGSVEWDGPPS | CMR_{INT} |
| | PHWTLYSDGSSSILSTNNATFQNTGTYRCTEPGDPL | CSF1R extracellular |
| | GGSAAIHLYVKDPARPWNVLAQEVVVFEDQDALL | domain CMR_{INT} |
| | PCLLTDPVLEAGVSLVRVRGRPLMRHTNYSFSPWH | (Uniprot P07333, aa 20- |
| | GFTIHRAKFIQSQDYQCSALMGGRKVMSISIRLKVQ | 502, 510-517) |
| | KVIPGPPALTLVPAELVRIRGEAAQIVCSASSVDVNF | |
| | DVFLQHNNTKLAIPQQSDFHNNRYQKVLTLNLDQV | |
| | DFQHAGNYSCVASNVQGKHSTSMFFRVVESAYLN | |
| | LSSEQNLIQEVTVGEGLNLKVMVEAYPGLQGFNWT | |
| | YLGPFSDHQPEPKLANATTKDTYRHTFTLSLPRLKP | |
| | SEAGRYSFLARNPGGWRALTFELTLRYPPEVSVIWT | |
| | FINGSGTLLCAASGYPQPNVTWLQCSGHTDRCDEA | |
| | QVLQVWDDPYPEVLSQEPFHKVTVQSLLTVETLEH | |
| | NQTYECRAHNSVGSGSWAFIPISPDEFLFTP | |
| 4 | RVETATETAW | CMR_{INT} |
| | | c-MPL hinge (Uniprot |
| | | P40238-1, aa 482-491) |
| 5 | ISLVTALHLVLGLSAVLGLLLL | CMR_{INT} |
| | | c-MPL transmembrane |
| | | (Uniprot P40238-1, aa |
| | | 492-513) |
| 6 | RWQFPAHYRRLRHALWPSLPDLHRVLGQYLRDTA | CMR_{INT} |
| | ALSPPKATVSDTCEEVEPSLLEILPKSSERTPLPLCSS | c-MPL juxtamembrane |
| | QAQMDYRRLQPSCLGTMPLSVCPPMAESGSCCTTH | and cytoplasmic domain |
| | IANHSYLPLSYWQQP | (Uniprot P40238-1, aa |
| | | 514-635) |
| 18 | MEFGLSWLFLVAILKGVQCIPVIEPSVPELVVKPGA | CMR_{INT} |
| | TVTLRCVGNGSVEWDGPPSPHWTLYSDGSSSILSTN | (full construct) |
| | NATFQNTGTYRCTEPGDPLGGSAAIHLYVKDPARP | |
| | WNVLAQEVVVFEDQDALLPCLLTDPVLEAGVSLV | |
| | RVRGRPLMRHTNYSFSPWHGFTIHRAKFIQSQDYQ | |
| | CSALMGGRKVMSISIRLKVQKVIPGPPALTLVPAEL | |
| | VRIRGEAAQIVCSASSVDVNFDVFLQHNNTKLAIPQ | |
| | QSDFHNNRYQKVLTLNLDQVDFQHAGNYSCVASN | |
| | VQGKHSTSMFFRVVESAYLNLSSEQNLIQEVTVGE | |
| | GLNLKVMVEAYPGLQGFNWTYLGPFSDHQPEPKL | |
| | ANATTKDTYRHTFTLSLPRLKPSEAGRYSFLARNPG | |
| | GWRALTFELTLRYPPEVSVIWTFINGSGTLLCAASG | |
| | YPQPNVTWLQCSGHTDRCDEAQVLQVWDDPYPEV | |
| | LSQEPFHKVTVQSLLTVETLEHNQTYECRAHNSVG | |
| | SGSWAFIPISPDEFLFTPRVETATETAWISLVTALHL | |
| | VLGLSAVLGLLLLRWQFPAHYRRLRHALWPSLPDL | |
| | HRVLGQYLRDTAALSPPKATVSDTCEEVEPSLLEIL | |
| | PKSSERTPLPLCSSQAQMDYRRLQPSCLGTMPLSVC | |
| | PPMAESGSCCTTHIANHSYLPLSYWQQP | |
| 1 | MEFGLSWLFLVAILKGVQC | CMR_{SHORT} |
| | | Signal peptide (derived |
| | | from an |
| | | immunoglobulin IgG1) |
| 19 | IPVIEPSVPELVVKPGATVTLRCVGNGSVEWDGPPS | CMR_{SHORT} |
| | PHWTLYSDGSSSILSTNNATFQNTGTYRCTEPGDPL | CSF1R extracellular |
| | GGSAAIHLYVKDPARPWNVLAQEVVVFEDQDALL | domain (Uniprot |
| | PCLLTDPVLEAGVSLVRVRGRPLMRHTNYSFSPWH | P07333, aa 20-502) |
| | GFTIHRAKFIQSQDYQCSALMGGRKVMSISIRLKVQ | |
| | KVIPGPPALTLVPAELVRIRGEAAQIVCSASSVDVNF | |
| | DVFLQHNNTKLAIPQQSDFHNNRYQKVLTLNLDQV | |
| | DFQHAGNYSCVASNVQGKHSTSMFFRVVESAYLN | |
| | LSSEQNLIQEVTVGEGLNLKVMVEAYPGLQGFNWT | |
| | YLGPFSDHQPEPKLANATTKDTYRHTFTLSLPRLKP | |
| | SEAGRYSFLARNPGGWRALTFELTLRYPPEVSVIWT | |
| | FINGSGTLLCAASGYPQPNVTWLQCSGHTDRCDEA | |
| | QVLQVWDDPYPEVLSQEPFHKVTVQSLLTVETLEH | |
| | NQTYECRAHNSVGSGSWAFIPIS | |
| 4 | RVETATETAW | CMR_{SHORT} |
| | | c-MPL hinge (Uniprot |
| | | P40238-1, aa 482-491) |
| 5 | ISLVTALHLVLGLSAVLGLLLL | CMR_{SHORT} |
| | | c-MPL transmembrane |
| | | (Uniprot P40238-1, aa |
| | | 492-513) |
| 6 | RWQFPAHYRRLRHALWPSLPDLHRVLGQYLRDTA | CMR_{SHORT} |
| | ALSPPKATVSDTCEEVEPSLLEILPKSSERTPLPLCSS | c-MPL juxtamembrane |
| | QAQMDYRRLQPSCLGTMPLSVCPPMAESGSCCTTH | and cytoplasmic domain |
| | IANHSYLPLSYWQQP | (Uniprot P40238-1, aa |
| | | 514-635) |
| 20 | MEFGLSWLFLVAILKGVQCIPVIEPSVPELVVKPGA | CMR_{SHORT} |
| | TVTLRCVGNGSVEWDGPPSPHWTLYSDGSSSILSTN | |
| | NATFQNTGTYRCTEPGDPLGGSAAIHLYVKDPARP | |
| | WNVLAQEVVVFEDQDALLPCLLTDPVLEAGVSLV | |
| | RVRGRPLMRHTNYSFSPWHGFTIHRAKFIQSQDYQ | |
| | CSALMGGRKVMSISIRLKVQKVIPGPPALTLVPAEL | |
| | VRIRGEAAQIVCSASSVDVNFDVFLQHNNTKLAIPQ | |
| | QSDFHNNRYQKVLTLNLDQVDFQHAGNYSCVASN | |
| | VQGKHSTSMFFRVVESAYLNLSSEQNLIQEVTVGE | |
| | GLNLKVMVEAYPGLQGFNWTYLGPFSDHQPEPKL | |
| | ANATTKDTYRHTFTLSLPRLKPSEAGRYSFLARNPG | |
| | GWRALTFELTLRYPPEVSVIWTFINGSGTLLCAASG | |
| | YPQPNVTWLQCSGHTDRCDEAQVLQVWDDPYPEV | |
| | LSQEPFHKVTVQSLLTVETLEHNQTYECRAHNSVG | |
| | SGSWAFIPISRVETATETAWISLVTALHLVLGLSAVL | |
| | GLLLLRWQFPAHYRRLRHALWPSLPDLHRVLGQY | |
| | LRDTAALSPPKATVSDTCEEVEPSLLEILPKSSERTP | |
| | LPLCSSQAQMDYRRLQPSCLGTMPLSVCPPMAESG | |
| | SCCTTHIANHSYLPLSYWQQP | |
| 21 | ATGGAATTTGGCCTGAGCTGGCTGTTCCTGGTGG | CMR_{FL} Construct |
| | CCATCCTGAAGGGCGTGCAGTGCATCCCAGTGAT | |
| | AGAGCCCAGTGTCCCTGAGCTGGTCGTGAAGCCA | |
| | GGAGCAACGGTGACCTTGCGATGTGTGGGCAATG | |
| | GCAGCGTGGAATGGGATGGCCCCCCATCACCTCA | |
| | CTGGACCCTGTACTCTGATGGCTCCAGCAGCATC | |
| | CTCAGCACCAACAACGCTACCTTCCAAAACACGG | |
| | GGACCTATCGCTGCACTGAGCCTGGAGACCCCCT | |
| | GGGAGGCAGCGCCGCCATCCACCTCTATGTCAAA | |
| | GACCCTGCCCGGCCCTGGAACGTGCTAGCACAGG | |
| | AGGTGGTCGTGTTCGAGGACCAGGACGCACTACT | |
| | GCCCTGTCTGCTCACAGACCCGGTGCTGGAAGCA | |
| | GGCGTCTCGCTGGTGCGTGTGCGTGGCCGGCCCC | |
| | TCATGCGCCACACCAACTACTCCTTCTCGCCCTG | |
| | GCATGGCTTCACCATCCACAGGGCCAAGTTCATT | |
| | CAGAGCCAGGACTATCAATGCAGTGCCCTGATGG | |
| | GTGGCAGGAAGGTGATGTCCATCAGCATCCGGCT | |
| | GAAAGTGCAGAAAGTCATCCCAGGGCCCCCAGC | |
| | CTTGACACTGGTGCCTGCAGAGCTGGTGCGGATT | |
| | CGAGGGGAGGCTGCCCAGATCGTGTGCTCAGCCA | |
| | GCAGCGTTGATGTTAACTTTGATGTCTTCCTCCAA | |
| | CACAACAACACTAAGCTOGCAATCCCTCAACAAT | |
| | CTGACTTTCATAATAACCGTTACCAAAAAGTCCT | |
| | GACCCTCAACCTCGATCAAGTAGATTTCCAACAT | |
| | GCCGGCAACTACTCCTGCGTGGCCAGCAACGTGC | |
| | AGGGCAAGCACTCCACCTCCATGTTCTTCCGGGT | |
| | GGTAGAGAGTGCCTACTTGAACTTGAGCTCTGAG | |
| | CAGAACCTCATCCAGGAGGTGACCGTGGGGGAG | |
| | GGGCTCAACCTCAAAGTCATGGTGGAGGCCTACC | |
| | CAGGCCTGCAAGGTTTTAACTGGACCTACCTGGG | |
| | ACCCTTTTCTGACCACCAGCCTGAGCCCAAGCTT | |
| | GCTAATGCTACCACCAAGGACACATACAGGCAC | |
| | ACCTTCACCCTCTCTCTGCCCCGCCTGAAGCCCTC | |
| | TGAGGCTGGCCGCTACTCCTTCCTGGCCAGAAAC | |
| | CCAGGAGGCTGGAGAGCTCTGACGTTTGAGCTCA | |
| | CCCTTCGATACCCCCCAGAGGTAAGCGTCATATG | |
| | GACATTCATCAACGGCTCTGGCACCCTTTTGTGT | |
| | GCTGCCTCTGGGTACCCCCAGCCCAACGTGACAT | |
| | GGCTGCAGTGCAGTGGCCACACTGATAGGTGTGA | |
| | TGAGGCCCAAGTGCTGCAGGTCTGGGATGACCCA | |
| | TACCCTGAGGTCCTGAGCCAGGAGCCCTTCCACA | |
| | AGGTGACGGTGCAGAGCCTGCTGACTGTTGAGAC | |
| | CTTAGAGCACAACCAAACCTACGAGTGCAGGGC | |
| | CCACAACAGCGTGGGGAGTGGCTCCTGGGCCTTC | |
| | ATACCCATCTCTGCAGGAGCCCACACGCATCCCC | |
| | CGGATGAGTTCCTCTTCACACCAAGGGTGGAGAC | |
| | CGCCACCGAGACCGCCTGGATCTCCTTGGTGACC | |
| | GCTCTGCATCTAGTGCTGGGCCTCAGCGCCGTCC | |
| | TGGGCCTGCTGCTGCTGAGGTGGCAGTTTCCTGC | |
| | ACACTACAGGAGACTGAGGCATGCCCTGTGGCCC | |
| | TCACTTCCAGACCTGCACCGGGTCCTAGGCCAGT | |
| | ACCTTAGGGACACTGCAGCCCTGAGCCCGCCCAA | |
| | GGCCACAGTCTCAGATACCTGTGAAGAAGTGGA | |
| | ACCCAGCCTCCTTGAAATCCTCCCCAAGTCCTCA | |
| | GAGAGGACTCCTTTGCCCCTGTGTTCCTCCCAGG | |
| | CCCAGATGGACTACCGAAGATTGCAGCCTTCTTG | |
| | CCTGGGGACCATGCCCCTGTCTGTGTGCCCACCC | |
| | ATGGCTGAGTCAGGGTCCTGCTGTACCACCCACA | |
| | TTGCCAACCATTCCTACCTACCACTAAGCTATTG | |
| | GCAGCAGCCTTAA | |
| 22 | ATGGAATTTGGCCTGAGCTGGCTGTTCCTGGTGG | CMR_{INT} Construct |
| | CCATCCTGAAGGGCGTGCAGTGCATCCCAGTGAT | |
| | AGAGCCCAGTGTCCCTGAGCTGGTCGTGAAGCCA | |
| | GGAGCAACGGTGACCTTGCGATGTGTGGGCAATG | |
| | GCAGCGTGGAATGGGATGGCCCCCCATCACCTCA | |
| | CTGGACCCTGTACTCTGATGGCTCCAGCAGCATC | |
| | CTCAGCACCAACAACGCTACCTTCCAAAACACGG | |
| | GGACCTATCGCTGCACTGAGCCTGGAGACCCCCT | |
| | GGGAGGCAGCGCCGCCATCCACCTCTATGTCAAA | |
| | GACCCTGCCCGGCCCTGGAACGTGCTAGCACAGG | |
| | AGGTGGTCGTGTTCGAGGACCAGGACGCACTACT | |
| | GCCCTGTCTGCTCACAGACCCGGTGCTGGAAGCA | |
| | GGCGTCTCGCTGGTGCGTGTGCGTGGCCGGCCCC | |
| | TCATGCGCCACACCAACTACTCCTTCTCGCCCTG | |
| | GCATGGCTTCACCATCCACAGGGCCAAGTTCATT | |
| | CAGAGCCAGGACTATCAATGCAGTGCCCTGATGG | |
| | GTGGCAGGAAGGTGATGTCCATCAGCATCCGGCT | |
| | GAAAGTGCAGAAAGTCATCCCAGGGCCCCCAGC | |
| | CTTGACACTGGTGCCTGCAGAGCTGGTGCGGATT | |
| | CGAGGGGAGGCTGCCCAGATCGTGTGCTCAGCCA | |
| | GCAGCGTTGATGTTAACTTTGATGTCTTCCTCCAA | |
| | CACAACAACACTAAGCTCGCAATCCCTCAACAAT | |
| | CTGACTTTCATAATAACCGTTACCAAAAAGTCCT | |
| | GACCCTCAACCTCGATCAAGTAGATTTCCAACAT | |
| | GCCGGCAACTACTCCTGCGTGGCCAGCAACGTGC | |
| | AGGGCAAGCACTCCACCTCCATGTTCTTCCGGGT | |
| | GGTAGAGAGTGCCTACTTGAACTTGAGCTCTGAG | |
| | CAGAACCTCATCCAGGAGGTGACCGTGGGGGAG | |
| | GGGCTCAACCTCAAAGTCATGGTGGAGGCCTACC | |
| | CAGGCCTGCAAGGTTTTAACTGGACCTACCTGGG | |
| | ACCCTTTTCTGACCACCAGCCTGAGCCCAAGCTT | |
| | GCTAATGCTACCACCAAGGACACATACAGGCAC | |
| | ACCTTCACCCTCTCTCTGCCCCGCCTGAAGCCCTC | |
| | TGAGGCTGGCCGCTACTCCTTCCTGGCCAGAAAC | |
| | CCAGGAGGCTGGAGAGCTCTGACGTTTGAGCTCA | |
| | CCCTTCGATACCCCCCAGAGGTAAGCGTCATATG | |
| | GACATTCATCAACGGCTCTGGCACCCTTTTGTGT | |
| | GCTGCCTCTGGGTACCCCCAGCCCAACGTGACAT | |
| | GGCTGCAGTGCAGTGGCCACACTGATAGGTGTGA | |
| | TGAGGCCCAAGTGCTGCAGGTCTGGGATGACCCA | |
| | TACCCTGAGGTCCTGAGCCAGGAGCCCTTCCACA | |
| | AGGTGACGGTGCAGAGCCTGCTGACTGTTGAGAC | |
| | CTTAGAGCACAACCAAACCTACGAGTGCAGGGC | |
| | CCACAACAGCGTGGGGAGTGGCTCCTGGGCCTTC | |
| | ATACCCATCTCTCCGGATGAGTTCCTCTTCACACC | |
| | AAGGGTGGAGACCGCCACCGAGACCGCCTGGAT | |
| | CTCCTTGGTGACCGCTCTGCATCTAGTGCTGGGC | |
| | CTCAGCGCCGTCCTGGGCCTGCTGCTGCTGAGGT | |
| | GGCAGTTTCCTGCACACTACAGGAGACTGAGGCA | |
| | TGCCCTGTGGCCCTCACTTCCAGACCTGCACCGG | |
| | GTCCTAGGCCAGTACCTTAGGGACACTGCAGCCC | |
| | TGAGCCCGCCCAAGGCCACAGTCTCAGATACCTG | |
| | TGAAGAAGTGGAACCCAGCCTCCTTGAAATCCTC | |
| | CCCAAGTCCTCAGAGAGGACTCCTTTGCCCCTGT | |
| | GTTCCTCCCAGGCCCAGATGGACTACCGAAGATT | |
| | GCAGCCTTCTTGCCTGGGGACCATGCCCCTGTCT | |
| | GTGTGCCCACCCATGGCTGAGTCAGGGTCCTGCT | |
| | GTACCACCCACATTGCCAACCATTCCTACCTACC | |
| | ACTAAGCTATTGGCAGCAGCCTTAA | |
| 23 | ATGGAATTTGGCCTGAGCTGGCTGTTCCTGGTGG | CMR_{SHORT} Construct |
| | CCATCCTGAAGGGCGTGCAGTGCATCCCAGTGAT | |
| | AGAGCCCAGTGTCCCTGAGCTGGTCGTGAAGCCA | |
| | GGAGCAACGGTGACCTTGCGATGTGTGGGCAATG | |
| | GCAGCGTGGAATGGGATGGCCCCCCATCACCTCA | |
| | CTGGACCCTGTACTCTGATGGCTCCAGCAGCATC | |
| | CTCAGCACCAACAACGCTACCTTCCAAAACACGG | |
| | GGACCTATCGCTGCACTGAGCCTGGAGACCCCCT | |
| | GGGAGGCAGCGCCGCCATCCACCTCTATGTCAAA | |
| | GACCCTGCCCGGCCCTGGAACGTGCTAGCACAGG | |
| | AGGTGGTCGTGTTCGAGGACCAGGACGCACTACT | |
| | GCCCTGTCTGCTCACAGACCCGGTGCTGGAAGCA | |
| | GGCGTCTCGCTGGTGCGTGTGCGTGGCCGGCCCC | |
| | TCATGCGCCACACCAACTACTCCTTCTCGCCCTG | |
| | GCATGGCTTCACCATCCACAGGGCCAAGTTCATT | |
| | CAGAGCCAGGACTATCAATGCAGTGCCCTGATGG | |
| | GTGGCAGGAAGGTGATGTCCATCAGCATCCGGCT | |
| | GAAAGTGCAGAAAGTCATCCCAGGGCCCCCAGC | |
| | CTTGACACTGGTGCCTGCAGAGCTGGTGCGGATT | |
| | CGAGGGGAGGCTGCCCAGATCGTGTGCTCAGCCA | |
| | GCAGCGTTGATGTTAACTTTGATGTCTTCCTCCAA | |
| | CACAACAACACTAAGCTCGCAATCCCTCAACAAT | |
| | CTGACTTTCATAATAACCGTTACCAAAAAGTCCT | |
| | GACCCTCAACCTCGATCAAGTAGATTTCCAACAT | |
| | GCCGGCAACTACTCCTGCGTGGCCAGCAACGTGC | |
| | AGGGCAAGCACTCCACCTCCATGTTCTTCCGGGT | |
| | GGTAGAGAGTGCCTACTTGAACTTGAGCTCTGAG | |
| | CAGAACCTCATCCAGGAGGTGACCGTGGGGGAG | |
| | GGGCTCAACCTCAAAGTCATGGTGGAGGCCTACC | |
| | CAGGCCTGCAAGGTTTTAACTGGACCTACCTGGG | |
| | ACCCTTTTCTGACCACCAGCCTGAGCCCAAGCTT | |
| | GCTAATGCTACCACCAAGGACACATACAGGCAC | |
| | ACCTTCACCCTCTCTCTGCCCCGCCTGAAGCCCTC | |
| | TGAGGCTGGCCGCTACTCCTTCCTGGCCAGAAAC | |
| | CCAGGAGGCTGGAGAGCTCTGACGTTTGAGCTCA | |
| | CCCTTCGATACCCCCCAGAGGTAAGCGTCATATG | |
| | GACATTCATCAACGGCTCTGGCACCCTTTTGTGT | |
| | GCTGCCTCTGGGTACCCCCAGCCCAACGTGACAT | |
| | GGCTGCAGTGCAGTGGCCACACTGATAGGTGTGA | |
| | TGAGGCCCAAGTGCTGCAGGTCTGGGATGACCCA | |
| | TACCCTGAGGTCCTGAGCCAGGAGCCCTTCCACA | |
| | AGGTGACGGTGCAGAGCCTGCTGACTGTTGAGAC | |
| | CTTAGAGCACAACCAAACCTACGAGTGCAGGGC | |
| | CCACAACAGCGTGGGGAGTGGCTCCTGGGCCTTC | |
| | ATACCCATCTCTAGGGTGGAGACCGCCACCGAGA | |
| | CCGCCTGGATCTCCTTGGTGACCGCTCTGCATCTA | |
| | GTGCTGGGCCTCAGCGCCGTCCTGGGCCTGCTGC | |
| | TGCTGAGGTGGCAGTTTCCTGCACACTACAGGAG | |
| | ACTGAGGCATGCCCTGTGGCCCTCACTTCCAGAC | |
| | CTGCACCGGGTCCTAGGCCAGTACCTTAGGGACA | |
| | CTGCAGCCCTGAGCCCGCCCAAGGCCACAGTCTC | |
| | AGATACCTGTGAAGAAGTGGAACCCAGCCTCCTT | |
| | GAAATCCTCCCCAAGTCCTCAGAGAGGACTCCTT | |
| | TGCCCCTGTGTTCCTCCCAGGCCCAGATGGACTA | |
| | CCGAAGATTGCAGCCTTCTTGCCTGGGGACCATG | |
| | CCCCTGTCTGTGTGCCCACCCATGGCTGAGTCAG | |
| | GGTCCTGCTGTACCACCCACATTGCCAACCATTC | |
| | CTACCTACCACTAAGCTATTGGCAGCAGCCTTAA | |

In some embodiments, the transmembrane domain comprises a transmembrane domain from a protein selected from VEGFR2, VEGFR1, FGFR1, PDGFR, HGFR, IGFR, IL-81R (IL-8Ra/IL-8Rb), TGFβR1, IL-10R, CSF1R, SCFR/KIT/cKIT/CD117, IL-9R, IL-4R, IL-6R, IL-10R, Tie2/CD202, C-MPL/TPOR, CD44, RANK, FGFR1, FGFR2, FGFR3, and FGFR4, VLA-4, NOTCH1, NOTCH2, NOTCH3, and NOTCH4, GM-SCFR/CSF2R, G-CSFR/CSF3R, IL-1R, immune checkpoints (such as PD-1, PDL1, PDL2, CTLA-4, CD200R TIM3, LAG-3, 214, BTLA, CTLA4, TIM3, LAG3, PD1, TIGIT, LAIR1, FASLG, ID2, CD80, CD83, KLRG1, KLRD1, KLRC1, B7-1, B7-H1, CD160), prostaglandin receptors (such as EP1, EP2, EP3, EP4, IP, TP, DP1, DP2, FP, adenosine receptors (such as A1, A2A, A2B, and A3), BMB receptors (such as BMPR1A, BMPR1B, BMPR2, ActR-1A, ActR-2A, ActR-2B), and a variant or fragment thereof.

In some embodiments, the transmembrane domain comprises a transmembrane domain from a protein selected from a cytokine receptor, a receptor tyrosine kinase (RTK), a c-MPL receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226 TNF receptor family receptors (such as TRAF, TRAF2, TRAF3/CD40BP, TRAF3IP1, TRAF4, TRAF7, TRAP1, TNFR1/TNFRSF1A/CD120a, TRAP100/MED24, TNFR2/TNFRSF1B/CD120b, LTBR/TNFRSF3, OX40/TNFRSF4/CD134, CD40/TNFRSF5, DcR3/TNFRSF6B, CD27/TNFRSF7, CD30/TNFRSF8, 4-1BB/TNFRSF9/CD137, TRAIL R1/CD261/TNFRSF10A, TRAIL R2/CD262/TNFRSF10B, TRAILR3/TNFRSF10C, TRAIL R4/CD264/TNFRSF10D, TNFRSF11A, Osteoprotegerin/TNFRSF11B, TNFRSF12A/FN14/TWEAKR, TACI/TNFRSF13B(CD267), BAFFR/TNFRSF13C/CD268, HVEM/TNFRSF14/CD270, BCMA/TNFRSF17/CD269, GITR/TNFRSF18/CD357, RELT/TNFRSF19L, TNFRSF19/TROY, TNFRSF21/DR6, TNFRSF25/DR3/TNFRSF12), and a variant or fragment thereof.

In some embodiments, the transmembrane domain comprises a transmembrane domain of a c-MPL receptor.

In some embodiments, the transmembrane domain comprises an amino acid sequence having at least 80% (e.g., 80%, 85%, 90%, 91%, 92%, 93%, 94%, 95%, 96%, 97%, 98%, 99%) sequence identity to SEQ ID NO: 5 or comprises the amino acid sequence of SEQ ID NO: 5 (see Table 1).

In some embodiments, the intracellular signaling domain comprises an intracellular domain of a protein selected from a cytokine receptor, a receptor tyrosine kinase (RTK), a c-MPL receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226 TNF receptor family receptors (such as TRAF1, TRAF2, TRAF3/CD40BP, TRAF3IP1, TRAF4, TRAF7, TRAP1, TNFR1/TNFRSF1A/CD120a, TRAP100/MED24, TNFR2/TNFRSF1B/CD120b, LTBR/TNFRSF3, OX40/TNFRSF4/CD134, CD40/TNFRSF5, DcR3/TNFRSF6B, CD27/TNFRSF7, CD30/TNFRSF8, 4-1BB/TNFRSF9/CD137, TRAIL R1/CD261/TNFRSF10A, TRAILR2/CD262/TNFRSF10B, TRAILR3/TNFRSF10C, TRAIL R4/CD264/TNFRSF10D, TNFRSF11A, Osteoprotegerin/TNFRSF11B, TNFRSF12A/FN14/TWEAKR, TACI/TNFRSF13B(CD267), BAFFR/TNFRSF13C/CD268, HVEM/TNFRSF14/CD270, BCMA/TNFRSF17/CD269, GITR/TNFRSF18/CD357, RELT/TNFRSF19L, TNFRSF19/TROY, TNFRSF21/DR6, TNFRSF25/DR3/TNFRSF12), and a variant or fragment thereof.

In some embodiments, the intracellular signaling domain comprises an intracellular domain of a cytokine receptor. In some embodiments, the intracellular signaling domain comprises an intracellular domain of a c-MPL receptor.

In some embodiments, the intracellular signaling domain comprises an amino acid sequence having at least 80% (e.g., 80%, 85%, 90%, 91%, 92%, 93%, 94%, 95%, 96%, 97%, 98%, 99%) sequence identity to SEQ ID NO: 6 or comprises the amino acid sequence of SEQ ID NO: 6.

In some embodiments, the chimeric polypeptide further comprises a signaling peptide linked to the N-terminus of the extracellular ligand-binding domain. In some embodiments, the signaling peptide comprises an Igκ signaling peptide.

In some embodiments, the signaling peptide comprises an amino acid sequence having at least 80% (e.g., 80%, 85%, 90%, 91%, 92%, 93%, 94%, 95%, 96%, 97%, 98%, 99%) sequence identity to SEQ ID NO: 1 or comprises the amino acid sequence of SEQ ID NO: 1.

In some embodiments, the transmembrane domain is linked to the extracellular ligand-binding domain via a linker (e.g., a peptide linker), or the transmembrane domain is linked to the intracellular signaling domain via a linker (e.g., a peptide linker). In some embodiments, the linker connecting the transmembrane domain and the intracellular signaling domain comprises a juxtamembrane (JM) region.

In some embodiments, the linker comprises a linker domain from a protein selected from VEGFR2, VEGFR1, FGFR1, PDGFR, HGFR, IGFR, IL-8R (IL-8Ra/IL-8Rb), TGFβR1, IL-10R, CSF1R, SCFR/KIT/cKIT/CD117, IL-9R, IL-4R, IL-6R, IL-10R, Tie2/CD202, C-MPL/TPOR, CD44, RANK, FGFR1, FGFR2, FGFR3, FGFR4, VLA-4, NOTCH1, NOTCH2, NOTCH3, NOTCH4, GM-SCFR/CSF2R, G-CSFR/CSF3R, IL-1R, immune checkpoints (such as PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160), prostaglandin receptors (such as EP1, EP2, EP3, EP4, IP, TP, DP1, DP2, FP, adenosine receptors (such as A1, A2A, A2B, and A3), or BMB receptors (such as BMPR1A, BMPR1B, BMPR2, ActR-1A, ActR-2A, ActR-2B), and a variant or fragment thereof.

In some embodiments, the linker comprises a linker domain from a protein selected from a cytokine receptor, a receptor tyrosine kinase (RTK), a c-MPL receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2, IL-7, IL-10, IL-12, IL-15, IL-18, IL-23, Epo, CD27, CD28, ICOS, HVEM, LIGHT, CD40L, CD27, OX40, DR3, GITR, CD30, SLAM, CD2, 2B4, CD226 TNF receptor family receptors (such as TRAF1, TRAF2, TRAF3/CD40BP, TRAF3IP1, TRAF4, TRAF7, TRAP1, TNFR1/TNFRSF1A/CD120a, TRAP100/MED24, TNFR2/TNFRSF1B/CD120b, LTBR/TNFRSF3, OX40/TNFRSF4/CD134, CD40/TNFRSF5, DcR3/TNFRSF6B, CD27/TNFRSF7, CD30/TNFRSF8, 4-1BB/TNFRSF9/CD137, TRAIL R1/CD261/TNFRSF10A, TRAIL R2/CD262/TNFRSF10B, TRAILR3/TNFRSF10C, TRAIL R4/CD264/TNFRSF10D, TNFRSF11A, Osteoprotegerin/TNFRSF11B, TNFRSF12A/FN14/TWEAKR, TACIT/TNFRSF13B(CD267), BAFFR/TNFRSF13C/CD268, HVEM/TNFRSF14/CD270, BCMA/TNFRSF17/CD269, GITR/TNFRSF18/CD357, RELT/TNFRSF19L, TNFRSF19/TROY, TNFRSF21/DR6, or TNFRSF25/DR3/TNFRSF12), and a variant or fragment thereof.

The term “juxtamembrane domain” or “juxtamembrane region,” as used herein, refers to the region that connects the transmembrane domain (e.g., transmembrane helix) to the catalytic domain (e.g., tyrosine kinase domain).

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present disclosure. In some embodiments, linker moieties include, but are not limited to, chemical linker moieties, or for example, a peptide linker moiety (a linker sequence).

A peptide linker can range from 2 amino acids to 60 or more amino acids, and in some embodiments, a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length. In some embodiments, a peptide linker is at least 5 amino acids, at least 6 amino acids or at least 7 amino acids in length and optionally is up to 30 amino acids, up to 40 amino acids, up to 50 amino acids or up to 60 amino acids in length. In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker may be generated by de novo design and/or optimized in silico.

In some embodiments, the linker comprises polar (e.g., serine (S)) or charged (e.g., lysine (K)) residues. In some embodiments, the linker is a flexible linker, e.g., comprising one or more glycine (G) or serine (S) residues.

Examples of flexible linkers that can be used in the fusion protein of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., represented by [Ser]m[Gly]n, where m or n is an integer from 1 to 20 (e.g., 1, 2, 3, 4, 5, 6, 7, 8, 9, 10, 11, 12, 13, 14, 15, 16, 17, 18, 19, 20). Polyglycine linkers can suitably be used in the fusion protein of the disclosure. In some embodiments, a peptide linker comprises two or more consecutive glycines, represented by [Gly]n where n is an integer from 1 to 20 (e.g., 1, 2, 3, 4, 5, 6, 7, 8, 9, 10, 11, 12, 13, 14, 15, 16, 17, 18, 19, 20).

In some embodiments, the chimeric polypeptide comprises an amino acid sequence having at least 80% (e.g., 80%, 85%, 90%, 91%, 92%, 93%, 94%, 95%, 96%, 97%, 98%, 99%) sequence identity to SEQ ID NO: 7, 9, 11, 16, 18, or 20 or comprises the amino acid sequence of SEQ ID NO: 7, 9, 11, 16, 18, or 20.

As used herein, the term “variant” refers to a first molecule that is related to a second molecule (also termed a “parent” molecule). The variant molecule can be derived from, isolated from, based on or homologous to the parent molecule.

A variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99%, or more identical in amino acid sequence compared to the original amino acid sequence. Polypeptide variants include polypeptides comprising the entire parent polypeptide and further comprising additional fused amino acid sequences. Polypeptide variants also include polypeptides that are portions or subsequences of the parent polypeptide. For example, unique subsequences (e.g., as determined by standard sequence comparison and alignment techniques) of the polypeptides disclosed hemin are also encompassed by this disclosure. Polypeptide variants may include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide and yield functionally identical polypeptides, including additions of a non-functional peptide sequence. In other aspects, the variant polypeptides change the biological activity of the parent molecule. One skilled in the art will appreciate that many variants of the disclosed polypeptides are encompassed by this disclosure. A polynucleotide or polypeptide variant can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids, or less than about 250 amino acids in length.

In some embodiments, a variant of the chimeric polypeptide may include one or more conservative modifications. The variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications.

The term “homolog” or “homologous,” when used in reference to a polypeptide, refers to a high degree of sequence identity between two polypeptides, or to a high degree of similarity between the three-dimensional structure or to a high degree of similarity between the active site and the mechanism of action. In some embodiments, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

Also within the scope of this disclosure are the variants, mutants, and homologs with significant identity to the disclosed chimeric polypeptides. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of chimeric polypeptides described herein.

In some embodiments, the chimeric polypeptide may be conjugated or linked to a detectable tag or a detectable marker (e.g., a radionuclide, a fluorescent dye). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, MBP-tag (MBP: maltose-binding protein), Alfa-tag, Sun-tag, and Moon-tag. Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of the chimeric polypeptide. The detectable tag and the affinity tag may also be separated by one or more amino acids. In some embodiments, the detectable tag can be conjugated or linked to the chimeric polypeptide via a cleavable element. In the context of the present disclosure, the term “cleavable element” relates to peptide sequences that are susceptible to cleavage by chemical agents or enzymatic means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate,” “conjugation,” or “linked” as used herein refers to the attachment of two or more entities to form one entity. A conjugate encompasses both peptide-small molecule conjugates as well as peptide-protein/peptide conjugates.

b. Polynucleotides and Vectors

In another aspect, this disclosure provides a polynucleotide encoding a polypeptide described above. In some embodiments, the polypeptide can be encoded by a single nucleic acid or by a plurality (e.g., two, three, four, or more) nucleic acids. The nucleic acids of the disclosure can be DNA or RNA (e.g., mRNA).

In another aspect, the disclosure provides vectors comprising nucleic acids encoding chimeric polypeptides as described above. The nucleic acids may be present in a single vector or separate vectors that are present in the same host cell or separate host cell.

The term “vector” or “expression vector” is synonymous with “expression construct” and refers to a nucleic acid molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector may comprise an expression cassette. Expression vectors allow transcription of large amounts of stable mRNA. Once the expression vector is inside the target cell, the ribonucleic acid molecule or protein that is encoded by the gene is produced by the cellular transcription and/or translation machinery.

The vectors may comprise a polynucleotide that encodes an RNA (e.g., RNAi, ribozymes, miRNA, siRNA) that, when transcribed from the polynucleotides of the vector, will result in the accumulation of chimeric proteins on the plasma membranes of target cells. Vectors that may be used include, without limitation, lentiviral, HSV, and adenoviral vectors. Lentiviruses include, but are not limited to, HIV-1, HIV-2, SIV, FIV, and EIAV. Lentiviruses may be pseudotyped with the envelope proteins of other viruses, including, but not limited to VSV, rabies, Mo-MLV, baculovirus, and Ebola. Such vectors may be prepared using standard methods in the art.

In some embodiments, the vector is a recombinant AAV vector. AAV vectors we DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced, and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, which contains the rep gene involved in viral replication and expression of the viral genes; and the right-hand part of the genome, which contains the cap gene encoding the capsid proteins of the virus.

The application of AAV, for example, as a vector for gene therapy, has been rapidly developed in recent years. Wild-type AAV can infect, with a comparatively high titer, dividing or non-dividing cells, or tissues of a mammal, including human, and also can integrate into human cells at a specific site (on the long arm of chromosome 19) (Kotin, R. M., et al., Proc. Natl. Acad. Sci. USA 87: 2211-2215, 1990) (Samulski, R. J, et al., EMBO J. 10: 3941-3950, 1991 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in the literature, respectively named AAV1, AAV2, AAV3, AAV4, AAV5, AAV6, AAV7, AAV8, AAV9, AAV10, AAV11, AAV12, AAV13, AAV14, AAV15, AAV16, and recombinant variants thereof, wherein AAV5 is originally isolated from humans (Bantel-Schaal and H. zur Hausen. 1984. Virology 134: 52-63), while AAV1-4 and AAV6 are all found in the study of adenovirus (Ursula Bantel-Schaal, Hajo Delius and Harald zur Hausen. J. Virol. 1999, 73: 939-947).

AAV vectors may be prepared using standard methods in the art. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors,” the disclosures of which are herein incorporated by reference in their entireties. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See, e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entireties). These publications describe various AAV-derived constructs in which the rep and/or cap genes are deleted and replaced by a gene of interest and the use of these constructs for transferring the gene of interest in vitro (into cultured cells) or in vivo (directly into an organism). The replication-defective recombinant AAVs can be prepared by co-transfecting a plasmid containing the nucleic acid sequence of interest flanked by two AAV inverted terminal repeat (ITR) regions and a plasmid carrying the AAV encapsidation genes (rep and cap genes) into a cell line that is infected with a human helper virus (for example an adenovirus). The AAV recombinants that are produced are then purified by standard techniques.

In some embodiments, the vector(s) can be encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, also provided is a recombinant virus particle (recombinant because it contains a recombinant polynucleotide) comprising any of the vectors described herein. Methods of producing such particles are known in the art and are described in U.S. Pat. No. 6,596,535.

In some embodiments, the viral vector comprises an AAV vector, lentiviral vector, adenoviral vector, or a non-viral plasmid vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

In some embodiments, vectors can be derived from retroviruses, including avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)). Retroviral genomes have been modified for use as a vector (Cone & Mulligan, Proc. Natl. Acad. Sci., USA, 81:6349-6353, (1984)). Non-limiting examples of retroviruses include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T cell lymphotropic virus (HTLV), and simian T cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

In some embodiments, the vector comprises a retroviral vector or a lentiviral vector. In some embodiments, lentiviral and retroviral vectors may be packaged using their native envelope proteins or may be modified to be encapsulated with heterologous envelope proteins. Examples of envelope proteins include, but are not limited to, an amphotropic envelope, an ecotropic envelope, or a xenotropic envelope, or may be an envelope including amphotropic and ecotropic portions. The protein also may be that of any of the above-mentioned retroviruses and lentiviruses. Alternatively, the env proteins may be modified, synthetic or chimeric env constructs, or may be obtained from non-retro viruses, such as vesicular stomatitis virus and HVJ virus. Specific non-limiting examples include the envelope of Moloney Murine Leukemia Virus (MMLV), Rous Sarcoma Virus, Baculovirus, Jaagsiekte Sheep Retrovirus (JSRV) envelope protein, and the feline endogenous virus RD114; gibbon ape leukemia virus (GALV) envelope; baboon endogenous virus (BaEV) envelope; simian sarcoma-associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to, the HVJ virus envelope.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

c. Cells

The disclosure also provides host cells comprising a nucleic acid or a vector of the disclosure. In one embodiment, the host cells are genetically engineered to comprise one or more nucleic acids or vectors, as described herein. In one embodiment, the host cells are genetically engineered by using an expression cassette. The phrase “expression cassette” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The cell can be, but is not limited to, a eukaryotic cell, an insect cell, or a mammalian cell (e.g., a human cell). Suitable eukaryotic cells include, but are not limited to, Vero cells, HeLa cells, COS cells, CHO cells, HEK293 cells, BHK cells, and MDCKII cells. Suitable insect cells include, but are not limited to, Sf9 cells.

In some embodiments, the cell comprises an immune cell. In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell. In some embodiments, the T cell comprises a human T cell.

Lymphocytes are one subtype of white blood cells in the immune system. In some embodiments, lymphocytes may include tumor-infiltrating immune cells. Tumor-infiltrating immune cells consist of both mononuclear and polymorphonuclear immune cells (i.e., T cells, B cells, natural killer cells, macrophages, neutrophils, dendritic cells, mast cells, eosinophils, basophils, etc.) in variable proportions. In some embodiments, lymphocytes may include tumor-infiltrating lymphocytes (TILs). TILs are white blood cells that have left the bloodstream and migrated towards a tumor. TILs can often be found in the tumor stroma and within the tumor itself. In some embodiments, TILs are “young” T cells or minimally cultured T cells. In some embodiments, the young cells have a reduced culturing time (e.g., between about 22 to about 32 days in total). In some embodiments, the lymphocytes express CD27.

In some embodiments, lymphocytes may include peripheral blood lymphocytes (PBLs). In some embodiments, lymphocytes include T lymphocytes (T cells) and/or natural killer cells (NK cells).

In some embodiments, the lymphocytes may be autologous, allogeneic, syngeneic, or xenogeneic with respect to the subject. In some embodiments, the lymphocytes are autologous in order to reduce an immunoreactive response against the lymphocyte when reintroduced into the subject for immunotherapy treatment.

In some embodiments, the T cells are CD8+ T cells. In some embodiments, the T cells are CD4+ cells. In some embodiments, the NK cells are CD 16+ CD56+ and/or CD57+NK cells. NKs are characterized by their ability to bind and to kill cells that fail to express self MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

In some embodiments, the immune cell expresses a TCR(e.g., a recombinant TCR, natural TCR) or a chimeric antigen receptor (CAR).

As used herein, the term “T cell receptor” or “TCR” refers to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an a chain and a β chain. Approximately 5% of T cells have TCRs made up of γ/δ chains. TCRs are membrane-anchored heterodimers that are found as part of a complex with a CD3 chain molecule. Each chain comprises two extracellular domains: a variable (V) region and a constant (C) region, the latter of which is membrane-proximal. The variable domains of α chains and β chains consist of three hypervariable regions that are also referred to as the complementarity determining regions (CDRs). The CDRs, in particular CDR3, are primarily responsible for contacting antigens and thus define the specificity of the TCR, although CDR1 of the α chain can interact with the N-terminal part of the antigen. CDR1 of the β chain interacts with the C-terminal part of the peptide. TCRs are also characterized by a series of highly conserved disulfide bonds that link the two chains.

As used herein, a “chimeric antigen receptor” or “CAR” refers to an engineered receptor that confers or grafts specificity for an antigen onto an immune effector cell (e.g., a human T cell). A chimeric antigen receptor typically comprises an extracellular ligand-binding domain or moiety and an intracellular domain that comprises one or more stimulatory domains that transduce the signals necessary for T cell activation. In some embodiments, the extracellular ligand-binding domain or moiety can be in the form of single-chain variable fragments (scFvs) derived from a monoclonal antibody, which provide specificity for a particular epitope or antigen (e.g., an epitope or antigen preferentially present on the surface of a cancer cell or other disease-causing cell or particle). The extracellular ligand-binding domain can be specific for any antigen or epitope of interest.

In some embodiments, the TCR or CAR may bind to a tumor-associated antigen. As used herein, the term “antigen” is a molecule and/or substance that can generate peptide fragments that are recognized by a TCR and/or induce an immune response. An antigen may contain one or more “epitopes.” In some embodiments, the antigen has several epitopes. An epitope is recognized by a TCR, an antibody, or a lymphocyte in the context of an MHC molecule.

The terms “tumor-associated antigen” and “cancer antigen,” as used herein, refer to any molecule (e.g., protein, peptide, lipid, carbohydrate, etc.) solely or predominantly expressed or over-expressed by a tumor cell and/or a cancer cell, such that the antigen is associated with the tumor and/or the cancer. The TAA/cancer antigen can also be expressed by normal, non-tumor, or non-cancerous cells. However, in such a situation, the expression of the TAA/cancer antigen by normal, non-tumor, or non-cancerous cells is in some embodiments not as robust as the expression of the TAA/cancer antigen by tumor and/or cancer cells. Thus, in some embodiments, the tumor and/or cancer cells overexpress the TAA and/or express the TAA at a significantly higher level as compared to the expression of the TAA by normal, non-tumor, and/or non-cancerous cells. In some embodiments, the phosphopeptides are fragments of TAAs or TAAs themselves.

The TAA can be an antigen expressed by any cell of any cancer or tumor, including the cancers and tumors described herein. The TAA can be a TAA of only one type of cancer or tumor, such that the TAA is associated with or characteristic of only one type of cancer or tumor. Alternatively, the TAA can be characteristic of more than one type of cancer or tumor. For example, the TAA can be expressed by both breast and prostate cancer cells and not expressed at all by normal, non-tumor, or non-cancer cells.

Non-limiting examples of tumor-associated proteins from which tumor antigens (including neoantigens) can be identified include, e.g., 13HCG, 43-9F, 5T4, 791Tgp72, adipophilin, AIM-2, ALDH1A1, alpha-actinin-4, alpha-fetoprotein (“AFP”), ARTC1, B-RAF, BAGE-1, BCA225, BCLX (L), BCR-ABL fusion protein b3a2, beta-catenin, BING-4, brain glycogen phosphorylase, BTAA, c-met, CA-125, CA-15-3 (CA 27.29/BCAA), CA-19-9, CA-242, CA-50, CA-72-4, CALCA, CAM 17.1, CAM43, carcinoembryonic antigen (“CEA”), CASP-5, CASP-8, CD274, CD45, CD68\KP1, Cdc27, CDK12, CDK4, CDK 2A, CEA, CLPP, CO-029, COA-1, CPSF, CSNK1A1, CT-7, CT9/BRDT, CTAG1, CTAG2, CTpl 1, cyclin D1, Cyclin-A1, dek-can fusion protein, DK 1, E2A-PRL, EBNA, EF2, EFTUD2, Elongation factor 2, ENAH (hMena), Ep-CAM, EpCAM, EphA3, epithelial tumor antigen (“ETA”), Epstein Barr virus antigens, ETV6-AML1 fusion protein, EZH2, FGF5, FLT3-ITD, FN1, G250, G250/MN/CALX, Ga733 (EpCAM), GAGE-1,2,8, GAGE-3,4,5,6,7, GAS7, glypican-3, GnTV, gplOO, gplOO/Pmel 17, GPNMB, H-ras, H4-RET, HAUS3, Hepsin, HER-2/neu, HERV-K-MEL, HLA-A11, HLA-A2, HLA-DOB, HOM-MD-21, HOM-MD-397, Horn/Me 1-40, Horn/Mel-55, HPV E2, HPV E6, HPV E7, hsp70-2, HTgp-175, IDO1, IGF2B3, IGH-IGK, IL13Ralpha2, Intestinal carboxyl esterase, K-ras, Kallikrein 4, KIAAO205, KIF20A, KK-LC-1, KKLC1, KM-HN-1, KMHN1 also known as CCDC110, LAGE-1, LAGE-2, LB33/MUM-1, LDLR-fucosyltransferaseAS fusion protein, Lengsin, M-CSF, M344, MA-50, MAGE-1, MAGE-2, MAGE-3, MAGE-4, MAGE-5, MAGE-6, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, MAGE-B (MAGE-B 1-MAGE-B24), MAGE-C(MAGE-C1/CT7, CT10), MAGE-Cl, MAGE-C2, MAGE-Xp2 (MAGE-B2), MAGE-Xp3 (MAGE-B3), MAGE-Xp4 (MAGE-B4), malic enzyme, mammaglobin-A, MAPE, MART-1, MART-2, MATN, MC1R, MCSP, mdm-2, ME1, Melan-A/MART-1, Meloe, MG7-Ag, Midkine, MMP-2, MMP-7, MOV 18, MUC1, MUC5AC, mucin, MUM-1, MUM-2, MUM-3, MYL-RAR, Myosin, Myosin class I, N-ras, N-raw, NA88-A, NAG, NBU70K, neo-PAP, NFYC, nm-23H1, NuMa, NY-BR-1, NY-CO-1, NY-CO-2, NY-ESO1, NY-ESO-1/LAGE-2, OA1, OGT, OS-9, P polypeptide, p15(58), p16, p185erbB2, p180erbB-3, p53, PAP, PAX5, PBF, pml-RARalpha fusion protein, polymorphic epithelial mucin (“PEM”), PPP1R3B, PRAME, PRDX5, PSA, PSCA, PSMA, PTPRK, RAB38/NY-MEL-1, RAGE-1, RBAF600, RCAS1, RGSS, R oC, RNF43, RU2AS, SAGE, SART-1, SART-3, SCP-1, SDCCAG16, secernin 1, SIRT2, SNRPD1, SOX10, Spl7, SPA17, SSX-1, SSX-2, SSX-4, SSX-5, STEAP, survivin, SYT-SSX1 or -SSX2 fusion protein, TA-90 (Mac-2 binding protein\cyclophilin C-associated protein), TAAL6, TAG-1, TAG-2, TAG-72-4, TAGE, Telomerase, TERT, TGF-betaRII, TLP, TPBG, TPS TRAG-3, Triosephosphate isomerase, TRP-1, TRP-2, TRP-1/gp75, TRP-2, TRP2-INT2, TSP-180, TSP50, tyrosinase, tyrosinase (“TYR”), VEGF, WT1, XAGE-1b/GAGED2a, Kras, WT-1 antigen (in lymphoma and other solid tumors), ErbB receptors, Melan A (MARTI), gp 100, tyrosinase, TRP-V/gp 75, and TRP-2 (in melanoma); MAGE-1 and MAGE-3 (in bladder, head and neck, and non-small cell carcinoma); HPV EG and E7 proteins (in cervical cancer); Mucin (MUC-1) (in breast, pancreas, colon, and prostate cancers); prostate-specific antigen (PSA) (in prostate cancer); carcinoembryonic antigen (CEA) (in colon, breast, and gastrointestinal cancers), and such shared tumor-specific antigens as MAGE-2, MAGE4, MAGE-6, MAGE-10, MAGE-12, BAGE-1, CAGE-1,2,8, CAGE-3 TO 7, LAGE-1, NY-ESO-1/LAGE-2, NA-88, GnTV, TRP2-INT2. For example, antigenic peptides characteristic of tumors include those listed in Cancer Vaccines and Immunotherapy (2000) Eds Stern, Beverley and Carroll, Cambridge University Press, Cambridge, Cancer Immunology (2001), Kluwer Academic Publishers, The Netherlands, International Patent Application Publication No. WO 20000/020581 and U.S. Patent Application Publication No. 2010/0284965, and www.cancerimmunity.org/peptidedatabase/Tcellepitopes, which are each incorporated herein by reference in their entirety.

Additional examples of tumor-associated proteins may include BCMA, C-Met, CA9, CAIX, CD123, CD133, CD138, CD171, CD174, CD19, CD20, CD22, CD23, CD274, CD276, CD33, CD38, CD44, CD5, CD70, CEA, CEACAM5, CSPG4, EGFR, EGFRvIII, EPCAM, EphA2, ERBB2, FAP, FOLH1, FOLR1, GD2, GPC3, GPNMB, HER2, HPV16-E6, Ig a chain, IL13Ra2, ILIRAP, KDR, L1CAM, LeY, MAGEA3, MAGEA4, MARTI, Mesothelin, MET, MS4A1, MSLN, Muc1, MUC1, MUC16, NCAM1, NKG2D ligand, PD-L1, PDCD1, PR1, PROM1, PSCA, PSCA, PSMA, ROR1, ROR1, ROR1, SLAMF7, TEM1, TNFRSF8, ULBP1, ULBP2, VEGFR2, WT1, or Public neoantigens.

As used herein, the term “neoantigen” refers to a newly formed antigenic determinant that arises from a somatic mutation(s) and is recognized as “non-self.” A neoantigen can include a polypeptide sequence or a nucleotide sequence. A mutation can include a frameshift or non-frameshift indel, missense or nonsense substitution, splice site alteration (e.g., alternatively spliced transcripts), genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen (see, e.g., Liepe et al., Science; 354(6310):354-358 (2006), incorporated herein by reference in its entirety). A neoantigen can include a canonical antigen. A neoantigen can also include non-canonical antigen. Neoantigen can be tumor-specific.

In another aspect, this disclosure provides a method of preparing a modified immune cell. The method comprises: (a) obtaining a plurality of immune cells; (b) introducing a vector disclosed herein into the plurality of immune cells; (c) introducing a second vector comprising a nucleic acid sequence encoding a recombinant TCR or a CAR to obtain a plurality of modified immune cells; and (d) optionally expanding the plurality of modified immune cells in a cell culture medium.

In some embodiments, the method includes culturing or expanding modified immune cells, e.g., to allow for increased immunogenic activity (e.g., greater and/or longer activity). The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

Conditions appropriate for lymphocyte culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion). Examples of other additives for lymphocyte expansion include, but are not limited to, surfactant, piasmanate, pH buffers such as HEPES, and reducing agents such as N-cetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

Expansion of the immune cells may be carried out using the methods and conditions known in the art. In some embodiments, expansion of the immune cells is carried out according to the methods described in the International Application No. PCT/EP2018/080343.

As stated above, additional receptor(s) may be engineered into the modified immune cells expressing a CAR or a TCR, for example, to enhance a cell function (e.g., anti-tumor function).

In some embodiments, the additional receptor is a CAR. CARs typically have an antigen-binding domain that is fused to an intracellular signaling domain that is capable of activating or stimulating an immune cell. A CAR's antigen-binding domain can be derived from the sequence of a naturally occurring ligand (e.g., as in natural ligand-derived CARs). Alternatively, the CAR's antigen-binding domain may be composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). The scFv may be fused to a transmembrane domain and then to an intracellular signaling domain. The CAR can be a first-generation, second generation, or third-generation CAR. “First-generation” CARs include those that solely provide CD3z signals upon antigen binding. “Second-generation” CARs include those that provide both costimulation (e.g., CD28 or CD137) and activation (CD3z). “Third-generation” CARs include those that provide multiple costimulation (e.g., CD28 and CD137) and activation (CD3z). The CAR may specifically recognize a cancer antigen. In some embodiments, the cancer antigen may be selected from B cell maturation antigen (BCMA), CD5, CD7, CD19, CD20, CD22, CD33, CD70, CD74, CD79b, CD123, C-type lectin 1 (CLL-1), CD5, CEA, EGP-2, EGP-40, EpCAM, EphA2, erb-B2,3,4, FBP, Fetal acetylcholine receptor, folate receptor-a, GD2, GD3, HER2, hTERT, IL-13R-a2, KDR, K-light chain, LeY, LI cell, MAGE-A1, Mesothelin, MUC1, MUC16, mutant RAS specific TCRs, mutant TP53 specific TCRs, NKG2D ligands, NY-ESO-1, oncofetal antigen (h5T4), PSCA, PSMA, ROR1, TACI, TAG-72, VEGF-R2, and WT1.

In some embodiments, a neoantigen is a tumor-associated antigen that is subject-specific and is referred herein to as a “private neoantigen.” In other embodiments, a neoantigen appears across a patient population, and is referred to herein as a “public neoantigen.”

Modified immune cells expressing a TCR may be engineered to express and secrete a soluble protein or multiple soluble proteins. Soluble proteins may include, but are not limited to, cytokines, chemokines, growth factors, soluble receptors, ligands, antibodies, antibody fragments, and antigen binding domains and functional variants thereof.

Cytokines that may be expressed and/or secreted by the modified immune cells described herein include, but are not limited to, interleukin-2 (IL-2), interleukin-3 (IL-3), interleukin-6 (IL-6), interleukin-7 (IL-7), interleukin-9 (IL-9), interleukin-11 (IL-11), interleukin-12 (IL-12), interleukin-15 (IL-15), interleukin-17 (IL-17), interleukin-18 (IL-18), interleukin-21 (IL-21), interleukin-33 (IL-33), granulocyte macrophage colony stimulating factor (GM-CSF), interferon alpha (IFN-alpha or IFN-a), interferon beta (IFN-beta or IFN-b), interferon gamma (IFN-gamma or IFN-g), transforming growth factor-beta (TGF-b), CCL19 and erythropoietin. In some embodiments, the cytokine expressed by the modified immune cell is IL-2. In some embodiments, the cytokine expressed by the genetically engineered lymphoid cell is IFN-gamma.

Chemokines that may be expressed and/or secreted by the modified immune cells described herein include, but are not limited to, CXC-chemokines such as interleukin-8 (IL-8), neutrophil-activating protein-1 (NAP-1), neutrophil-activating protein-2 (NAP-2), GRO, GROp, GROγ, ENA-78, GCP-2, IP-10, MIG, CXCL1, CXCL12, CXCL16, CXCL19, and PF4; and CC chemokines, RANTES, MIR-1a, MIR-2b, monocyte chemotactic protein-1 (MCP-1), MCP-2, MCP-3, CCL5, and cotaxin. Suitable chemokines described in the International Publication No. WO2000078334A1 (e.g., Table 1), which is incorporated herein by reference in its entirety, are also contemplated by this disclosure.

Growth factors that may be expressed and/or secreted by the modified immune cells described herein include, but are not limited to, granulocyte macrophage-colony stimulating factor (GM-CSF), granulocyte-colony stimulating factor, macrophage-colony stimulating factor, tumor necrosis factor, transforming growth factors, epidermal growth factors, stem cell factor, platelet-derived growth factors, nerve growth factors, fibroblast growth factors, insulin-like growth factor, growth hormone, interleukin-1 (IL-1), interleukin-2 (IL-2), keratinocyte growth factor, ciliary neurotrophic growth factor, Schwann cell-derived growth factor, vaccinia virus growth factor, bombyxin, neu differentiation factor, v-Sis, and glial growth factor.

Soluble receptors that may be expressed and/or secreted by the modified immune cells described herein include, but are not limited to, soluble cytokine receptors such as IL-R1, IL-1RII, TNFR1, TNFRII, IFN-a/pR, IL-4 receptor, IL-6 receptor, IL-10 receptor, IL-11 receptor, IL-13 receptor, IL-18 binding protein, and TGF-b receptor; and soluble growth factor receptors such as soluble epidermal growth factor receptors (sEGFRs), soluble vascular endothelial growth factor receptors and PD-1 ectodomain, and soluble VEGFR-1 and SIRP-alpha molecules. Soluble receptors that may be expressed and/or secreted by the modified immune cells described herein may further be fused to CD28 endodomain or 4-1BB endodomain or any other co-stimulatory endodomains known in the art.

The cells can be engineered to express the peptide(s) and/or immunomodulators by any means known in the art, including, but not limited to, transfection, viral delivery (i.e., transduction), liposomal delivery, electroporation, cell squeeze (e.g., cells are first disrupted (e.g., squeezed, deformed, or compressed) followed by exposure to an applied energy field, e.g., an electric, magnetic, or acoustic field), injection, cationic polymer, a cationic lipid, calcium phosphate, and endocytosis.

In some embodiments, genetic engineering of immune cells may be accomplished by at least one of transfection, transduction, and temporary cell membrane disruption (i.e., cell squeeze) to introduce at least one polynucleotide encoding the chimeric polypeptide disclosed herein or a TCR or CAR into an immune cell. In some embodiments, the polynucleotide(s) are introduced into the immune cells by transducing a substantially homogeneous cell population with a recombinant expression vector. Such vectors may be a viral vector or a non-viral vector. Exemplary viral vectors for use in the invention include, but are not limited to, a retroviral vector (including lentiviral vectors), an adenoviral vector, an adeno-associated viral (AAV) vector, a herpes viral vector, or a baculoviral vector. In one embodiment, the viral vector for use in the invention is a lentiviral vector.

In some embodiments, electroporation can be used to permeabilize the cells by the application of electrostatic potential to the cell of interest. Cells subjected to an external electric field in this manner are subsequently predisposed to the uptake of exogenous nucleic acids. Electroporation of mammalian cells is described in detail, e.g., in Chu et al., Nucleic Acids Research 15: 131 1 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distder et al, Experimental Dermatology 14:315 (2005), as well as in US 2010/031714, the disclosures of each of which are incorporated herein by reference in their entirety. Additional techniques useful for the transfection of cells include the cell squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Cell squeeze-poration is described in detail, e.g., in Sharei et al., Journal of Visualized Experiments 81:e50980 (2013), the disclosure of which is incorporated herein by reference in its entirety.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig J., Topics in Current Chemistry 228:227 (2003), the disclosure of which is incorporated herein by reference) and diethylamino ethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406. The disclosure of this reference discussed above is incorporated herein by reference in its entirety.

Another useful tool for inducing the uptake of exogenous nucleic acids by the cell is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference in its entirety.

Microvesicles represent another potential vehicle that can be used to modify the genome of a cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as gesicles, for the genetic modification of eukaryotic cells is described in detail, e.g., in Quinn et al., Proceedings of the 18th Annual Meeting of the American Society of Gene and Cell Therapy; 2015 May 13, Abstract No. 122, the disclosure of which is incorporated herein by reference in its entirety.

Various methods may be used to transduce cells. In some embodiments, a cell is transduced with a vector or plasmid, i.e., a nucleic acid molecule capable of transporting a nucleic acid sequence between different cellular or genetic environments. Different cellular environments include different cell types of the same organism, while different genetic environments include cells of different organisms or other situations of cells with different genetic material and/or genomes. Non-limiting vectors of this disclosure include those capable of autonomous replication and expression of nucleic acid sequences (for delivery into the cell) present therein. Vectors may also be inducible for expression in a way that is responsive to factors specific for a cell type. Non-limiting examples include inducible by addition of an exogenous modulator in vitro or systemic delivery of vector inducing drugs in vivo. Vectors may also optionally comprise selectable markers that are compatible with the cellular system used. One type of vector for use in this disclosure is maintained as an episome, which is a nucleic acid capable of extra-chromosomal replication. Another type is a vector which is stably integrated into the genome of the cell in which it is introduced.

d. Compostions and Kits

In another aspect, the above-described chimeric polypeptide, nucleic acid, vector, or cell can be incorporated into compositions, e.g., pharmaceutical compositions suitable for administration.

The composition may further include a pharmaceutically acceptable carrier. The pharmaceutical compositions are generally formulated in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials that are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable and includes excipients that are acceptable for veterinary use as well as for human pharmaceutical use.

The term “pharmaceutically acceptable carrier” includes a pharmaceutically acceptable salt, pharmaceutically acceptable material, composition or carrier, such as a liquid or solid filler, diluent, excipient, solvent, or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water, isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer, and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion, and by the use of surfactants.

In some embodiments, the pharmaceutical compositions may include a population of modified immune cells produced by the methods described herein and a pharmaceutically acceptable carrier and/or excipient. In some embodiments, the pharmaceutical compositions may comprise substantially isolated/purified immune cells and a pharmaceutically acceptable carrier in a form suitable for administration to a subject. Pharmaceutically acceptable carriers are determined in part by the particular composition being administered, as well as by the particular method used to administer the composition. The pharmaceutical compositions we generally formulated in full compliance with all Good Manufacturing Practice (GMP) regulations of the U.S. Food and Drug Administration.

In some embodiments, the pharmaceutical composition further comprises a therapeutic agent. In some embodiments, the therapeutic agent comprises an anti-tumor or anti-cancer agent. In some embodiments, the additional agent comprises a pathway-targeted therapy, an immune-modulating therapy, or a tumor microenvironment-modulating therapy.

In some embodiments, the anti-tumor or anti-cancer agent is selected from the group consisting of taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof. In some embodiments, the therapeutic agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

In some embodiments, the disclosed pharmaceutical compositions can also include adjuvants such as aluminum salts and other mineral adjuvants, tensioactive agents, bacterial derivatives, vehicles, and cytokines. Adjuvants can also have antagonizing immunomodulating properties. For example, adjuvants can stimulate Th1 or Th2 immunity. Compositions and methods as disclosed herein can also include adjuvant therapy.

In some embodiments, the pharmaceutical compositions can be formulated in any conventional manner using one or more physiologically acceptable carriers and/or excipients. The lymphocytes may be formulated for administration by, for example, injection, parenteral, vaginal, rectal administration, or by administration directly to a tumor.

In some embodiments, the pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. In some embodiments, the pharmaceutical compositions can further be formulated as suspensions, solutions, or emulsions in oily or aqueous vehicles and may contain other agents, including suspending, stabilizing and/or dispersing agents.

In some embodiments, the pharmaceutical forms suitable for injectable use can include sterile aqueous solutions or dispersions; formulations including sesame oil, peanut oil, or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g., refrigeration and freezing) and must be preserved against the contaminating action of microorganisms, such as bacteria and fungi.

If formulations disclosed herein are used as a therapeutic to boost immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A carrier can also be a solvent or dispersion medium containing, for example, water, saline, polyol (for example, glycerol, propylene glycol, and liquid polyethylene glycol, and the like), suitable mixtures thereof, and vegetable oils. The prevention of the action of microorganisms can be brought about by various antibacterial and antifungal agents known in the art. In many cases, it will be preferable to include isotonic agents, for example, sugars or sodium chloride.

In some embodiments, the above-described chimeric polypeptides, nucleic acids, vectors, cells, or the composition (e.g., the pharmaceutical composition) can be provided in a kit. In one embodiment, the kit includes a container that contains an agent comprising the above-described chimeric polypeptides, nucleic acids, vectors, cells or the composition, and optionally informational material. The informational material can be descriptive, instructional, marketing, or other material that relates to the methods described herein and/or the use of the agents for therapeutic benefit. For example, kits may include instruction for the manufacturing, for the therapeutic regimen to be used, and periods of administration. In an embodiment, the kit includes also includes an additional therapeutic agent (e.g., a checkpoint modulator, a chemotherapeutic compound). The kit may comprise one or more containers, each with a different reagent. For example, the kit includes a first container that contains the above-described chimeric polypeptides, nucleic acids, vectors, cells, or the composition and a second container for the additional therapeutic agent. In some embodiments, the containers can include a unit dosage of the pharmaceutical composition. In addition to the composition, the kit can include other ingredients, such as a solvent or buffer, an adjuvant, a stabilizer, or a preservative.

In some embodiments, the kit optionally includes a device suitable for administration of the composition, e.g., a syringe or other suitable delivery device. The device can be provided pre-loaded with one or both of the agents or can be empty but suitable for loading.

### Methods of Treatment and T Cell Therapy

In another aspect, this disclosure additionally provides a method of treating cancer in a subject in need thereof. The method comprises administering to the subject a therapeutically effective amount of a cell or a composition, as disclosed herein.

In another aspect, this disclosure provides a method of treating cancer in a subject in need thereof, comprising: (a) obtaining a plurality of immune cells; (b) introducing a vector disclosed herein into the plurality of immune cells; (c) introducing a second vector comprising a nucleic acid sequence encoding a recombinant TCR or a CAR to obtain a plurality of modified immune cells; and (d) administering to the subject a therapeutically effective amount of the plurality of modified immune cells.

In some embodiments, the vector as disclosed herein may be introduced to an endogenous repertoire of polyclonally expanded tumor directed T cells (e.g., TCR T cells, CAR T cells, TILs, or tumor associated antigen specific T cells generated in cultures with peptide pulsed antigen presenting cells, etc.)

In some embodiments, the method further comprises administering to the patient an additional agent or therapy. In some embodiments, the additional agent comprises an anti-tumor or anti-cancer agent. In some embodiments, the additional agent comprises a pathway targeted therapy, an immune modulating therapy, or a tumor microenvironment modulating therapy.

In some embodiments, the additional agent or therapy comprises an autologous or allogeneic hematopoietic stem cell transplantation, for example, as used in treating hematologic malignancies, e.g., multiple myeloma, leukemia. In some embodiments, the additional agent or therapy comprises bispecific T cell engager (BiTEs) targeting, for example, BCMA, GPRC5D, etc for treating, e.g., myeloma.

In some embodiments, the additional agent or therapy comprises one or more cytokines, such as IL2, IL7, IL15, IL21, etc.

In some embodiments, the additional agent or therapy is administered before, after, or concurrently with the composition.

In some embodiments, the anti-tumor or anti-cancer agent is selected from taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin, an oncolytic virus, and combinations thereof. In some embodiments, the anti-tumor or anti-cancer agent comprises a chemotherapeutic agent selected from the group consisting of asparaginase, busulfan, carboplatin, cisplatin, daunorubicin, doxorubicin, fluorouracil, gemcitabine, hydroxyurea, methotrexate, paclitaxel, rituximab, vinblastine, and vincristine.

The immune cells (e.g., T cells) used in T cell therapy (e.g., adoptive therapy) can be harvested from a variety of sites, including peripheral blood, malignant effusions, resected lymph nodes, and tumor biopsies. Although T cells harvested from the peripheral blood are easier to obtain technically, TILs obtained from biopsies may contain a higher frequency of tumor-reactive cells. Once harvested, T cells can be transfected with a vector as described above.

In some embodiments, the immune cells are autologous or allogeneic. In some embodiments, immune cells can be harvested from one or more healthy donors, when generating an allogeneic immune cell product.

As used herein, the term “subject” may be interchangeably used with the term “patient.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

In some embodiments, the subject is a human. In some embodiments, the subject has cancer.

As used herein, the terms “treating,” “treat,” or the like mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject.

As used herein, “cancer,” “tumor,” and “malignancy,” all relate equivalently to hyperplasia of a tissue or organ. If the tissue is a part of the lymphatic or immune system, malignant cells may include non-solid tumors of circulating cells. Malignancies of other tissues or organs may produce solid tumors. The methods described herein can be used in the treatment of lymphatic cells, circulating immune cells, and solid tumors.

Cancers that can be treated include tumors that are not vascularized or are not substantially vascularized, as well as vascularized tumors. Cancers may comprise non-solid tumors (such as hematologic tumors, e.g., multiple myeloma, leukemias, and lymphomas) or may comprise solid tumors. The types of cancers to be treated with the disclosed compositions include, but are not limited to, carcinoma, blastoma and sarcoma, and certain leukemias or malignant lymphoid tumors, benign and malignant tumors and malignancies, e.g., sarcomas, carcinomas, and melanomas. Also included are adult tumors/cancers and pediatric tumors/cancers.

Hematologic cancers are cancers of the blood or bone marrow. Examples of hematologic (or hematogenous) cancers include leukemias, including acute leukemias (such as acute lymphocytic leukemia, acute myelocytic leukemia, acute myelogenous leukemia, promyelocytic, myelomonocytic, monocytic, and erythroleukemia), chronic leukemias (such as chronic myelocytic (granulocytic) leukemia, chronic myelogenous leukemia, and chronic lymphocytic leukemia), myeloproliferative neoplasms, polycythemia vera, essential thrombocythemia, chronic idiopathic myelofibrosis, lymphoma, Hodgkin's disease, non-Hodgkin's lymphoma (indolent and high-grade forms, of B or T cell lineage), multiple myeloma, Waldenstrom's macroglobulinemia, heavy chain disease, myelodysplastic syndromes, hairy cell leukemia.

Solid tumors are abnormal masses of tissue that usually do not contain cysts or liquid areas. Solid tumors can be benign or malignant. The different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma and other sarcomas, synovium, mesothelioma, Ewing tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, carcinoma of the sweat gland, medullary thyroid carcinoma, papillary thyroid carcinoma, sebaceous gland carcinoma of pheochromocytomas, carcinoma papillary, papillary adenocarcinomas, medullary carcinoma, bronchogenic carcinoma, renal cell carcinoma, hepatoma, bile duct carcinoma, choriocarcinoma, Wilms tumor, cervical cancer, testicular tumor, seminoma, bladder carcinoma, melanoma, and CNS tumors (such as glioma) (such as brainstem glioma and mixed gliomas), glioblastoma (also astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastasis).

Non-limiting examples of tumors can be treated by the methods described herein include, for example, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endothelio sarcoma, lymphangiosarcoma, lymphangioendotheliosarcoma, synovioma, testicular cancer, uterine cancer, cervical cancer, gastrointestinal cancer, mesothelioma, cancers associated with viral infection (such as but not limited to human papilloma virus (IHPV) associated tumors (e.g., cancer cervix, vagina, vulva, head and neck, anal, and penile carcinomas)), Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma.

Cancers that may be treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lympho epithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor, nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglio neuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythro leukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, lymphoma, melanoma, meningioma, multiple neuroendocrine type I and type II tumors, multiple myeloma, myelodysplastic syndromes, myeloproliferative diseases, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

The anti-tumor responses after treatment by the methods disclosed herein may be determined in xenograft tumor models. Tumors may be established using any human cancer cell line expressing the TAAs presented by the viral particles. In order to establish xenograft tumor models, about 5×10⁶ viable cells, may be injected, e.g., s.c, into NSG mice (NOD-SCID-gamma chain knockout) using, for example, Matrigel (Becton Dickinson). The endpoint of the xenograft tumor models can be determined based on the size of the tumors, weight of animals, survival time, and histochemical and histopathological examination of the cancer, using methods known to one skilled in the art.

In some embodiments, the subject is immunodepleted prior to treatment with the composition. For example, the subject can be pro-treated with non-myeloablative chemotherapy prior to an infusion of lymphocytes generated by the methods described herein. In one embodiment, a population of immune cells can be administered by infusion. In one embodiment, the non-myeloablative chemotherapy can be cyclophosphamide 60 mg/kg/d for 2 days (days 27 and 26 prior to antigen-specific lymphocyte infusion) and fludarabine 25 mg/m2/d for 5 days (days 27 to 23 prior to antigen-specific lymphocyte infusion). In one embodiment, after non-myeloablative chemotherapy and antigen-specific lymphocyte infusion (at day 0), according to the present disclosure, the subject can receive an intravenous infusion of IL-2 intravenously at 720,000 IU/kg every 8 hours to physiologic tolerance. In some embodiments, the population of immune cells can be used for treating cancer in combination with IL-2, wherein the IL-2 is administered after the population of immune cells.

In some embodiments, the composition, e.g., a population of immune cells, is administered with an additional therapeutic agent or therapy. In some embodiments, the composition can be administered to a subject either simultaneously with, before (e.g., 1-30 days before) or after (e.g., 1-30 days after) the additional therapeutic (including but not limited to small molecules, antibodies, or cellular reagents) that acts to elicit an immune response (e.g., to treat cancer) in the subject. When co-administered with an additional therapeutic, the composition and the additional therapeutic agent may be administered simultaneously or sequentially (in any order). Suitable therapeutically effective dosages for each agent may be lowered due to the additive action or synergy.

In some embodiments, the methods described herein can be combined with additional immunotherapies and therapies. For example, when used for treating cancer, the composition can be used in combination with conventional cancer therapies, such as, e.g., surgery, radiotherapy, chemotherapy or combinations thereof, depending on type of the tumor, patient condition, other health issues, and a variety of factors. In some embodiments, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof(e.g., anti-hVEGF antibody A4.6.1, bevacizumab or ranibizumab).

Non-limiting examples of chemotherapeutic compounds which can be used in combination treatments include, for example, aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-aspargine which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker, nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In some embodiments, the composition can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can also be combined with other treatments that possess the ability to modulate NKT function or stability, including but not limited to CD 1d, CD 1d-fusion proteins, CD 1d dimers or larger polymers of CD id either unloaded or loaded with antigens, CD1d-chimeric antigen receptors (CD1d-CAR), or any other of the five known CD1 isomers existing in humans (CD 1a, CD 1b, CD1c, CD1e), in any of the aforementioned forms or formulations, alone or in combination with each other or other agents.

The pharmaceutical compositions, as described, can be administered in a manner appropriate to the disease to be treated or prevented. The amount and frequency of administration will be determined by factors such as the condition of the patient, and the type and severity of the patient's disease, although appropriate dosages can be determined by clinical trials.

When “a therapeutically effective amount,” “an immunologically effective amount,” “an effective antitumor quantity,” or “an effective tumor-inhibiting amount” is indicated, the precise amount of the compositions of the present disclosure to be administered can be determined by a physician having account for individual differences in age, weight, tumor size, extent of infection or metastasis, and patient's condition. It can generally be stated that a pharmaceutical composition comprising the lymphocytes described herein can be administered at a dose of 10⁴ to 10⁹ cells/kg body weight, e.g., 10⁵ to 10⁶ cells/kg body weight, including all values integers within these intervals. The lymphocyte compositions can also be administered several times at these dosages. The cells can be administered using infusion techniques that are commonly known in immunotherapy (see, for example, Rosenberg et al., New Eng. J. of Med. 319: 1676, 1988). The optimal dose and treatment regimen for a particular patient can be readily determined by one skilled in the art of medicine by monitoring the patient for signs of the disease and adjusting the treatment accordingly.

In some embodiments, the composition can be administered to the subject in a manner compatible with the dosage formulation and in such amount as is therapeutically effective. Dose ranges and frequency of administration can vary depending on, e.g., the nature of the population of cells (e.g., immune cells) produced by the methods described herein and the medical condition as well as parameters of a specific patient and the route of administration used.

In some embodiments, the population of cells, e.g., immune cells, produced by the methods described herein can be administered to a subject at a dose ranging from about 10⁷ to about 10¹¹. A more accurate dose can also depend on the subject in which it is being administered. For example, a lower dose may be required if the subject is juvenile, and a higher dose may be required if the subject is an adult human subject. In some embodiments, a more accurate dose can depend on the weight of the subject.

The administration of the compositions as disclosed can be carried out in any convenient way, including infusion or injection (i.e., intravenous, intrathecal, intramuscular, intraluminal, intratracheal, intraperitoneal, or subcutaneous), tranadermal administration, or other methods known in the art. Administration can be once every two weeks, once a week, or more often, but the frequency may be decreased during a maintenance phase of the disease or disorder. In some embodiments, the composition is administered by intravenous infusion.

In some embodiments, the cells, e.g., immune cells, are activated and expanded using the methods described herein or other methods known in the art, wherein the cells are expanded to therapeutic levels, before administering to a patient together with (e.g., before, simultaneously or after) any number of relevant treatment modalities.

Also described herein, the compositions can be used in combination with chemotherapy, radiation, immunosuppressive agents, such as cyclosporin, azathioprine, methotrexate, mycophenolate, and FK506, antibodies, or other immunoablating agents such as CAMPATH, anti-cancer antibodies. CD3 or other antibody therapies, cytoxine, fludarabine, cyclosporine, FK506, rapamycin, mycophenolic acid, steroids, FR901228, cytokines, and irradiation.

In some embodiments, the compositions can also be administered to a patient together with (e.g., before, simultaneously, or after) bone marrow transplantation, therapy with T lymphocyte ablation using chemotherapy agents such as fludarabine, radiation therapy external beam (XRT), cyclophosphamide, or antibodies such as OKT3 or CAMPATH. Also described herein, the compositions can be administered after ablative therapy of B lymphocytes, such as agents that react with CD20, for example, Rituxan. For example, subjects may undergo standard treatment with high-dose chemotherapy, followed by transplantation of peripheral blood stem cells. In some embodiments, after transplantation, the subjects receive an infusion of the expanded lymphocytes, or the expanded lymphocytes are administered before or after surgery.

### Methods of Constructing Allosteric Biosensors

In yet another aspect, this disclosure provides a method of constructing a chimeric receptor. The method comprises: (a) selecting an extracellular ligand-binding domain and an intracellular signaling domain for the chimeric receptor from a plurality of extracellular ligand-binding domains and intracellular signaling domains based on a predetermined input signal and output function to be coupled by the chimeric receptor; (b) selecting a transmembrane domain from a plurality of transmembrane domains for linking the extracellular ligand-binding and the intracellular signaling domain; (c) self-associating three-dimensional structures of the extracellular ligand-binding domain, the intracellular signaling domain, and the transmembrane domain by docking; (d) linking and assembling subunit structures into an ensemble of chimeric receptor structures; (e) energy minimizing the oligomeric receptor models and selecting a subset of energy-minimized oligomeric receptor models having low energy by clustering the energy-minimized oligomeric receptor models based on an energy function; (f) calculating stability of the subset of the energy-minimized oligomeric receptor models upon binding to the input signal, wherein a level of stability corresponds to a degree of ligand-induced oligomerization of the chimeric receptor, (g) calculating a level of long-range mechanical dynamic coupling between the extracellular ligand-binding domain and the intracellular signaling domain of the subset of the energy-minimized oligomeric receptor models; (h) ranking the subset of the energy-minimized oligomeric receptor models based on the level of stability thereof and the level of long-range mechanical dynamic coupling between the extracellular ligand-binding domain and the intracellular signaling domain; and (i) selecting an optimal oligomeric receptor model with specific levels of stability and/or levels of long-range mechanical dynamic coupling between the extracellular ligand-binding domain and the intracellular signaling domain.

In some embodiments, the method further comprises repeating step (a) to step (i) for a plurality of combinations of extracellular ligand-binding domains, intracellular signaling domains, and transmembrane domains; and selecting an optimal oligomeric receptor model from the plurality of combinations with specific levels of stability and/or levels of long-range mechanical dynamic coupling between the extracellular ligand-binding domain and the intracellular signaling domain.

As used herein, the term “docking” refers to manipulation or placement of a molecular entity (e.g., protein) in a binding pocket or in close proximity to another molecular entity to understand molecular interactions. For example, docking can include orienting, rotating, translating a molecular entity in a binding pocket, domain, molecule or molecular complex or portion thereof based on distance geometry and/or energy. In some embodiments, docking can be performed by distance geometry methods that find sets of atoms of a molecular entity that match sets of sphere centers of the binding pocket, domain, molecule or molecular complex or portion thereof. See Meng et al. J. Comp. Chem. 4: 505-524 (1992). Sphere centers are generated by providing an extra radius of given length from the atoms (excluding hydrogen atoms) in the binding pocket, domain, molecule or molecular complex or portion thereof. Real-time interaction energy calculations, energy minimizations, or rigid-body minimizations (Gschwend et al., J. Mol. Recognition 9:175-186 (1996)) can be performed while orienting the chemical entity to facilitate docking. Docking can also be performed by combining a Monte Carlo search technique with rapid energy evaluation using molecular affinity potentials. See Goodsell and Olson, Proteins: Structure, Function and Genetics 8:195-202 (1990). Software programs that carry out docking functions include but are not limited to MATCHMOL (Cory et al., J. Mol. Graphics 2: 39 (1984); MOLFIT (Redington, Comput. Chem. 16 217 (1992)) and DOCK (Meng et al., supra).

In some embodiments, the method further comprises, prior to step (e), de novo designing an inter-domain linker connecting the extracellular ligand-binding domain and the transmembrane domain or an inter-domain linker connecting the transmembrane domain and the intracellular signaling domain. “De novo design,” as used herein, refers to construction of one or more linkers entirely through computer simulation.

In some embodiments, the method further comprises, prior to step (e), de novo designing the transmembrane domain.

In some embodiments, step (e) comprises energy minimizing the oligomeric receptor models by a Monte Carlo Minimization with simulated annealing. “Monte Carlo Minimization methods” or “Monte Carlo” methods are a broad class of computational algorithms that rely on repeated random sampling to obtain numerical results. The underlying concept is to use randomness to solve problems that might be deterministic in principle. They are often used in physical and mathematical problems and are most useful when it is difficult or impossible to use other approaches. Monte Carlo methods are mainly used in three problem classes: optimization, numerical integration, and generating draws from a probability distribution (Kroese, D. P., et al. WIREs Comput Stat. 6 (6): 386-392(2014)).

Simulated annealing (SA) is a probabilistic technique for approximating the global optimum of a given function (Kirkpatrick, S., et al. Science. 220 (4598): 671-680.). Specifically, it is a metaheuristic to approximate global optimization in a large search space for an optimization problem. It is often used when the search space is discrete (for example, the traveling salesman problem, the boolean satisfiability problem, protein structure prediction, and job-shop scheduling). For problems where finding an approximate global optimum is more important than finding a precise local optimum in a fixed amount of time, simulated annealing may be preferable to exact algorithms such as gradient descent or branch and bound.

In some embodiments, step (g) comprises calculating the level of long-range mechanical dynamic coupling between the extracellular ligand-binding domain and the intracellular signaling domain using a Rosetta force field and an Elastic Network model. The Rosetta force field models protein structures at the atomic level and enables the calculation of binding stabilities between protein chains. Elastic Network models approximate and enable very fast calculation of protein structural dynamics.

In some embodiments, step (g) comprises self-associating the transmembrane domain using EFDOCK-TM (Wang, Y., et al. Nat Commun 6, 7196(2015)). EFDOCK-TM is a prediction method of TMH oligomeric structures that is guided by sequence coevolutionary information. It can model TM structure either from sequence information alone or starting from the structure of a homolog.

In some embodiments, three-dimensional structures of extracellular ligand-binding domains, transmembrane domains, and intracellular signaling domains are generated by homology modeling. The term “generating a three-dimensional structure” or “generating a three-dimensional representation” refers to converting the lists of structure coordinates into structural models or graphical representations in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations. The term “homology model” refers to a structural model derived from known three-dimensional structure(s). Generation of the homology model, termed “homology modeling,” can include sequence alignment, residue replacement, residue conformation adjustment through energy minimization, or a combination thereof.

In some embodiments, the input signal is present in a tumor microenvironment associated with cancer. In some embodiments, the cancer comprises a solid tumor. In some embodiments, the cancer comprises a hematologic malignancy.

In some embodiments, the output function comprises an anti-tumor function. In some embodiments, the anti-tumor function comprises: (i) secretion of one or more cytokines from an immune cell, (ii) co-stimulation of the immune cell, (iii) cell survival of the immune cell, (iv) proliferation of the immune cell, (v) migration of the immune cell, (vi) modified metabolism of the immune cell, (vii) altered differentiation status of the immune cell, (viii) functionality of the immune cell, or a combination thereof.

In some embodiments, the input signal comprises a soluble factor that is enriched in a tumor microenvironment. In some embodiments, the input signal comprises a growth factor, cytokine, or interleukin. In some embodiments, the input signal comprises vascular endothelial growth factor (VEGF), basic fibroblast growth factor (bFGF), platelet-derived growth factor (PDGF), hepatocyte growth factor (HGF), insulin-like growth factor (IGF), interleukin-8 (IL-8), TGFβ, IL-10, or colony stimulating factor 1 (CSF-1), interleukin-34 (IL-34), stem cell factor (SCF), interleukin-9 (IL-9), interleukin-4 (IL-4), interleukin-6 (IL4), interleukin-10 (IL-10), Angiopoietin-1 (Ang1) (receptor is Tie2, or CD202), Thrombopoietin (TPO), Osteopontin (OPN), Receptor activator of nuclear factor kappa beta (NFkB) ligand, RANK ligand (RANKL), Fibroblast growth factor (FGF-1, -2), Vascular cell adhesion protein 1 (VCAM-1), Notch ligands: Jagged1, Jagged2, Delta-like1, Delta-like3, Delta-like4, GM-SCF/CSF2, G-CSF/CSF3, IL-1b, MMPs (such as MMP-1, MMP-2, MMP-3, MMP-7, MMP-8, MMP-9, MMP-13, MMP-14, TIMP-1, TIMP-2, TIMP-3, or TIMP-4), prostaglandin ligands (such as PGD2, PGE2, PGF2α, PGI2, TXA2, or PGH2), BMPs (such as: BMP1, BMP2, BMP3, BMP4, BMP5, BMP6, BMP7, BMP8, BMP9, BMP10, BMP11, BMP12, BMP13, BMP14, or BMP15), or adenosine receptors agonists (such as N6-Cyclopentyladenosine, N6-3-methoxyl-4-hydroxybenzyl adenine riboside (B2), adenosine, CCPA, Certain Benzodiazepines and Barbiturates, 2′-MeCCPA, GR 79236, SDZ WAG 994, Benzyloxy-cyclopentyladenosine (BnOCPA), N6-3-methoxyl-4-hydroxybenzyl adenine riboside (B2), ATL-146e, CGS-21680, Regadenoson, adenosine, 5′-N-ethylcarboxamidoadenosine, BAY 60-6583, adenosine, LUF-5835, LUF-5845, 2-(1-Hexynyl)-N-methyladenosine, CF-101 (IB-MECA), Adenosine, 2-Cl-IB-MECA, CP-532,903, or MRS-3558).

In some embodiments, the extracellular ligand-binding domain comprises one or more extracellular domains of VEGFR2, VEGFR1, FGFR1, PDGFR, HGFR, IGFR, IL-8R (IL-8Ra/IL-8Rb), TGFβR1, IL-10R, CSF1R, SCFR/KIT/cKIT/CD117, IL-9R, IL-4R, IL-6R, IL-10R, Tie2/CD202, C-MPL/TPOR, CD44, RANK, FGFR1, FGFR2, FGFR3, and FGFR4, VLA-4, NOTCH1, NOTCH2, NOTCH3, and NOTCH4, GM-SCFR/CSF2R, G-CSFR/CSF3R, IL-1R, immune checkpoints (such as PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160), prostaglandin receptors (such as EP1, EP2, EP3, EP4, IP, TP, DP1, DP2, FP, adenosine receptors (such as A1, A2A, A2B, and A3), BMB receptors (such as BMPR1A, BMPR1B, BMPR2, ActR-IA, ActR-2A, ActR-2B), or a variant or fragment thereof.

In some embodiments, the extracellular ligand-binding domain comprises one or more extracellular domains of VEGFR2 or variants thereof. In some embodiments, the extracellular ligand-binding domain comprises one or more extracellular domains of a CSF-1 receptor (CSF-1R) or variants thereof or specific linker sequences.

In some embodiments, the extracellular ligand-binding domain comprises D1-7; D1-4 and D7; or D1-3 extracellular domains of the VEGFR2.

In some embodiments, the transmembrane domain comprises a transmembrane domain from a protein selected from a cytokine receptor, a receptor tyrosine kinase (RTK), a c-MPL receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226 TNF receptor family receptors (such as TRAF1, TRAF2, TRAF3/CD40BP, TRAF3IP1, TRAF4, TRAF7, TRAP1, TNFR1/TNFRSF1A/CD120a, TRAP100/MED24, TNFR2/TNFRSF1B/CD120b, LTBR/TNFRSF3, OX40/TNFRSF4/CD134, CD40/TNFRSF5, DcR3/TNFRSF6B, CD27/TNFRSF7, CD30/TNFRSF8, 4-1BB/TNFRSF9/CD137, TRAIL R1/CD261/TNFRSF10A, TRAIL R2/CD262/TNFRSF10B, TRAILR3/TNFRSF10C, TRAIL R4/CD264/TNFRSF10D, TNFRSF11A, Osteoprotegerin/TNFRSF11B, TNFRSF12A/FN14/TWEAKR, TACI/TNFRSF13B(CD267), BAFFR/TNFRSF13C/CD268, HVEM/TNFRSF14/CD270, BCMA/TNFRSF17/CD269, GITR/TNFRSF18/CD357, RELT/TNFRSF19L, TNFRSF19/TROY, TNFRSF21/DR6, TNFRSF25/DR3/TNFRSF12), and a variant or fragment thereof.

In some embodiments, the transmembrane domain comprises a transmembrane domain of a c-MPL receptor.

In some embodiments, the intracellular signaling domain comprises an intracellular domain of a protein selected from a cytokine receptor, a receptor tyrosine kinase (RTK), a c-MPL receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226 TNF receptor family receptors (such as TRAF1, TRAF2, TRAF3/CD40BP, TRAF3IP1, TRAF4, TRAF7, TRAP1, TNFRI/TNFRSF1A/CD120a, TRAP100/MED24, TNFR2/TNFRSF1B/CD120b, LTBR/TNFRSF3, OX40/TNFRSF4/CD134, CD40/TNFRSF5, DcR3/TNFRSF6B, CD27/TNFRSF7, CD30/TNFRSF8, 4-1BB/TNFRSF9/CD137, TRAIL R1/CD261/TNFRSF10A, TRAILR2/CD262/TNFRSF10B, TRAILR3/TNFRSF10C, TRAIL R4/CD264/TNFRSF10D, TNFRSF11A, Osteoprotegerin/TNFRSF11B, TNFRSF12A/FN14/TWEAKR, TACI/TNFRSF13B(CD267), BAFFR/TNFRSF13C/CD268, HVEM/TNFRSF14/CD270, BCMA/TNFRSF17/CD269, GITR/TNFRSF18/CD357, RELT/TNFRSF19L, TNFRSF19/TROY, TNFRSF21/DR6, TNFRSF25/DR3/TNFRSF12), and a variant or fragment thereof.

In some embodiments, the intracellular signaling domain comprises an intracellular domain of a cytokine receptor. In some embodiments, the intracellular signaling domain comprises an intracellular domain of a c-MPL receptor.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “chimeric” or “heterologous” refers to two components that are defined by structures derived from different sources or progenitor sequences. For example, where “heterologous” is used in the context of a chimeric polypeptide, the chimeric polypeptide can include operably linked amino acid sequences that can be derived from different polypeptides of different phylogenic groupings.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

In some embodiments, the polynucleotide may include a codon-optimized sequence. For example, the nucleotide sequence encoding the chimeric polypeptide variant/fragment may be codon-optimized for expression in a eukaryote or eukaryotic cell. In some embodiments, the codon-optimized chimeric polypeptide variant/fragment is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et at. Nucl. Acids Res. 28:292 (2000). Computer algorithms for codon optimizing a particular sequence for expression in a particular host cell are also available, such as Gene Forge (Aptagen; Jacobus, Pa.). As to codon usage in yeast, reference is made to the online Yeast Genome database available at http//www.yeastgenome.org/community/codonusage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al., Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below. The percent identity between two amino acid sequences can be determined using the algorithm of E. Meyers and W. Miller (Comput. Appl. Biosci., 4:11-17 (1988)) which has been incorporated into the ALIGN program, using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” if the polynucleotide is derived from genomic DNA, expression may include splicing of the mRNA in a eukaryotic cell.

In some embodiments, the nucleotide sequence is operably linked to a promoter. The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions in the same reading frame.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule. Or other functional disruption of, or functional addition to, the genetic material of the cell. Exemplary modifications include modifications in the coding region of a heterologous or homologous polypeptide derived from the reference or parent molecule or a functional fragment thereof.

As used herein, the phrase “specific binding” refers to binding between a TCR, TCR-like molecule, or antigen-binding fragment thereof and an antigen and/or an epitope thereof (including but not limited to a peptide, optionally in complex with an MHC molecule) that is indicative of the presence of the antigen and/or the epitope thereof. As such, a TCR, TCR-like molecule, or antigen-binding fragment thereof is said to “specifically” bind an antigen and/or an epitope thereof when the dissociation constant (Kd) is less than about 1 μM, less than about 100 nM, or less than about 10 nM. Interactions between a TCR, TCR-like molecule, or antigen-binding fragment thereof and an epitope can also be characterized by an affinity constant (Kₐ). In some embodiments, a Kₐ of less than about 10⁷/M is considered “high affinity.”

The terms “T cell” and “T lymphocyte” are interchangeable and used synonymously herein. As used herein, T-cell includes thymocytes, naïve T lymphocytes, immature T lymphocytes, mature T lymphocytes, resting T lymphocytes, or activated T lymphocytes. A T-cell can be a T helper (Th) cell, for example, a T helper 1 (Th1) or a T helper 2 (Th2) cell. The T-cell can be a helper T-cell (HTL; CD4⁺ T-cell) CD4⁺ T-cell, a cytotoxic T-cell (CTL; CD8⁺ T-cell), a tumor-infiltrating cytotoxic T-cell (TIL; CD8⁺ T-cell), CD4⁺CD8⁺ T-cell, or any other subset of T-cells. Other illustrative populations of T-cells suitable for use in particular embodiments include naïve T-cells and memory T-cells. Also included are “NKT cells,” which refer to a specialized population of T-cells that express a semi-invariant ab T-cell receptor, but also express a variety of molecular markers that are typically associated with NK cells, such as NK1.1. NKT cells include NK1.1⁺ and NK1. G, as well as CD4⁺, CD4, CD8⁺, and CD8 cells. The TCR on NKT cells is unique in that it recognizes glycolipid antigens presented by the MHC I-like molecule CD 1d. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated a- and b-TCR chains, the TCR in γδ T-cells is made up of a g- chain and a d-chain. γδ T-cells can play a role in immunosurveillance and immunoregulation and were found to be an important source of IL-17 and to induce robust CD8⁺ cytotoxic T-cell response. Also included are “regulatory T-cells” or “Tregs” which refer to T-cells that suppress an abnormal or excessive immune response and play a role in immune tolerance. Tregs cells are typically transcription factor Foxp3-positive CD4⁺ T cells and can also include transcription factor Foxp3-negative regulatory T-cells that are IL-10-producing CD4C T cells.

The terms “natural killer cell” and “NK cell” are used interchangeably and used synonymously herein. As used herein, NK cell refers to a differentiated lymphocyte with a CD 16+CD56+ and/or CD57+TCR− phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician. Thus, the term “treatment” includes preventing a condition from occurring in a patient, particularly when the patient is predisposed to acquiring the condition; reducing and/or inhibiting the condition and/or its development and/or progression; and/or ameliorating and/or reversing the condition. Insofar as some embodiments of the methods of the presently disclosed subject matter are directed to preventing conditions, it is understood that the term “prevent” does not require that the condition be completely thwarted. Rather, as used herein, the term “preventing” refers to the ability of one of ordinary skill in the art to identify a population that is susceptible to the condition, such that administration of the compositions of the presently disclosed subject matter might occur prior to the onset of the condition. The term does not imply that the condition must be completely avoided.

The term “inhibiting cell growth” or “inhibiting proliferation of cells” refers to reducing or halting the growth rate of cells. For example, by inhibiting the growth of tumor cells, the rate of increase in size of the tumor may slow. In other embodiments, the tumor may stay the same size or decrease in size, i.e., regress. In particular embodiments, the rate of cell growth or cell proliferation is inhibited by at least 20%, at least 30%, at least 40%, at least 50%, at least 60%, at least 70%, at least 80%, or at least 90%.

The terms “transformation” and “transfection” refer to the directed modification of the genome of a cell by the external application of purified recombinant DNA from another cell of different genotype, leading to its uptake and integration into the subject cell's genome. In bacteria, the recombinant DNA is not typically integrated into the bacterial chromosome, but instead replicates autonomously as a plasmid. The terms “transformed” and “transfected” are used interchangeably herein. For example, a T cell may be transfected with a DNA sequence encoding a modified or high affinity TCR described herein prior to adoptive T cell treatment.

The term “eliciting” or “enhancing” in the context of an immune response refers to triggering or increasing an immune response, such as an increase in the ability of immune cells to target and/or kill cancer cells or to target and/or kill pathogens and pathogen-infected cells (e.g., EBV-positive cancer cells).

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit]“per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder or pathological condition; reducing or preventing the onset of a disease, symptom, disorder or condition; and generally counteracting a disease, symptom, disorder or pathological condition.

As used herein, “administering” refers to the physical introduction of a composition comprising a therapeutic agent to a subject, using any of the various methods and delivery systems known to those skilled in the art. Example routes of administration for antibodies described herein include intravenous, intraperitoneal, intramuscular, subcutaneous, intratumoral, intravesical, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

“Combination” therapy, as used herein, unless otherwise clear from the context, is meant to encompass administration of two or more therapeutic agents in a coordinated fashion and to includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

“Parenteral” administration of a composition includes, e.g., subcutaneous (s.c.), intravenous (i.v.), intramuscular (i.m.), or intrasternal injection, or infusion techniques.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Chimeric Receptor Modeling and Design.

An automated computational approach was developed to rationally engineer customized chimeric cell surface receptors that artificially couple extracellular ligand sensing to unrelated intracellular signaling outputs. In principle, prospective users require only their domains of choice as PDB inputs, in addition to a local Rosetta and Python 3.x installation to be used in conjunction with the purpose-built command line program and Python module. The Python module relies on Biobox (Lucas S P Rudden, et al., Bioinformatics, Volume 38, Issue 4, February 2022, Pages 1149-1151) for manipulating and preparing PDB metadata under the hood. All tools described here are available as part of the Rosetta software. Pre- and post-processing for all assembly steps can be completed with the command line scripts and associated Python module available with documentation on GitHub (https://github.com/barth-lab/domain_assembly_constraints). A detailed tutorial/manual detailing how users can construct their own chimeras is also provided.

The method first models and assembles combinations of protein domains to build oligomeric receptor structures that span the extracellular (EC), transmembrane (TM), and intracellular (IC) regions of the cell. It then ranks chimeric receptor structures based on: (a) the level of receptor oligomer stabilization upon ligand binding, and (b) the mechanical coupling between the ligand binding site and the signaling domains that correlate with allosteric signal transduction capabilities. The approach combines different modeling tools such as EFDOCK-TM to model de novo oligomeric TM domain structures, as well as comparative, loop modeling and docking techniques from the Rosetta software. To enable proper assembly of multi-domain receptor oligomeric structures, Rosetta's mp_domain_assembly method was further developed as described below.

### 1. Development of a Domain Assembly Protocol for Modeling Oligomeric Receptor Structures.

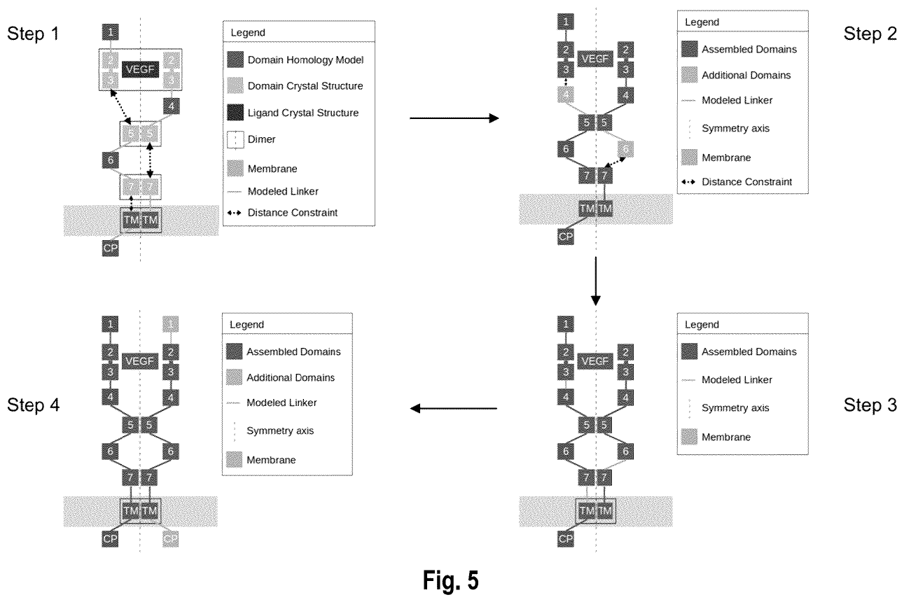

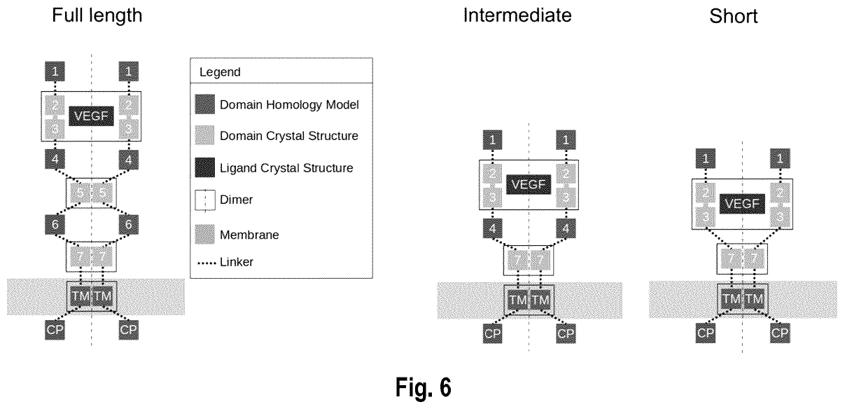

Rosetta's original mp_domain_assembly was developed to assemble multi-domain receptor monomeric structures. However, receptor monomeric units are usually not functional and membrane receptors often assemble into oligomeric structures to bind ligands and signals. To build receptor dimer or higher oligomeric structures, a number of operations were implemented, which enable the assembly and docking of oligomeric domain structures into the receptor scaffold. The protocol also allows the modeling to be guided by a wide variety of experimental constraints ranging from homolog structures to sparse conserved interactions between domains (see examples for a dimer in FIGS. 5 and 6).

### 2. Engineering and Selection of Optimal Receptor Chimeras.

The number of assembly steps is dictated by the combination of domains to dock and assemble and by the topology of the receptor scaffold. A detailed example of the protocol for building the most complex chimera, VEGFR2-c-MPL dimer receptor with 14 EC, 2 TM, and 2 IC domains is described below. In general, what is described herein also applies to any dimeric receptor design case. A typical assembly step for linking domains together requires 10,000 independent simulation trajectories where globular domains are represented and move as rigid bodies while protein regions linking the domains are constructed de novo using peptide fragment insertion techniques followed by energy minimization over all protein conformational degrees of freedom performed by a Monte Carlo Minimization with the Simulated Annealing protocol. Assembled receptor structures were refined using a relaxation protocol, and the lowest 10% energy models were clustered to identify the most representative conformations. In the above-mentioned modeling steps, receptor conformations were scored using an energy function developed for membrane protein structure prediction and design.

The best scoring structures of the 20 largest clusters were then selected for further analysis. First, the stabilization of the receptor dimeric structure upon ligand binding was calculated for each model. A high level of stabilization can be interpreted as a strong ligand-induced activation of the receptor. Second, the information transfer between the ligand binding EC and the TM regions that reflect the potential for signal transduction between the ligand and the signaling domains were approximated by the coupled motions between pairs of residues from each domain and were calculated using normal mode analysis implemented in the Rosetta protocol. Briefly, conformational dynamic fluctuations of all receptor residues were extracted from the 20 lowest frequency calculated normal modes of motion. Cross-correlation matrices were then calculated to extract correlation in conformational fluctuations (i.e., coupled movements) between pairs of residues located in the ligand binding or the TM domains. The correlations have no unit, and their magnitude can vary between 0 (no correlation in residue movement) to +1.0 (completely correlated movements). A high level of dynamic correlation can be interpreted as a strong potential for signal transduction mediated by the chimeric receptor across the cell surface.

### 3. Modeling and Assembly of VEGF-c-MPL Chimera.

Chimeras were created by assembling the TM and IC regions of the native c-MPL receptor with distinct combinations of IgG domains connecting the TM domain with the native VEGF binding domain of the VEGFR2 receptor. Since VEGFR2 and c-MPL function as ligand-bound dimers, the chimera was modeled in a dimeric form.

Individual domain structures were modeled as follows:

### TM Domain:

In the absence of experimental structure, the TM domain was modeled as a symmetric dimer starting from the homolog monomeric TM structure of EpoR using the software EFDOCK-TM (Wang, Y.& Barth. Nat Commun 6, 7196(2015)). 10,000 models were generated, and the top 10% lowest energy structures were clustered. The lowest energy conformations from the two largest clusters were analyzed to identify the residues mediating critical contacts at the dimer interface. The active-like conformation that is the most consistent with experimental mutagenesis studies of the c-MPL dimerization was selected for subsequent receptor modeling starting from the assembly with the cytoplasmic region.

### Cytoplasmic Domain:

The cytoplasmic signaling domain recruits and activates JAK kinases. It is composed of 2 structured switch motifs connected by a partially disordered region and was modeled by homology to that of the related EpoR receptor bound to JAK2 (pdb code: 6E2Q). 10′000 models were generated and clustered. A large number of clusters were obtained that reflect the high level of conformational flexibility of that region. The lowest energy conformation from the largest cluster was selected for assembly with the TM domain.

### Extracellular Domains:

Several IgG domains of VEGF receptors were considered to design the extracellular regions of the chimera. The N-terminal domain D1 was modeled by homology to the D1 structure of VEGFR3 (4bsk). The dimeric form of the domains 2 and 3 bound to VEGF was taken from the corresponding X-ray structure (2×1w). Missing loops were rebuilt onto the X-ray structure of VEGFR2 domains 4 and 5 (5oyj) to generate the complete monomeric assembled structure of these domains. Since domain 5 is known to form stable homodimers, domain 5 monomers were docked to obtain homodimeric conformations guided by the homodimer structure of VEGFR3 D5. Domain 6 was modeled by homology to IgG domain structure 3mj6. Domain 7 was modeled starting from the corresponding VEGFR2 D7 homodimer X-ray structure (3kvq).

### Assembly of the Chimeric Receptors:

The first assembly step consisted in linking the TM dimer with the cytoplasmic regions to create an entire dimeric signaling TM+CT domain from the c-MPL receptor. Subsequent assembly steps depended on the combination of domains that were engineered in the EC region of the distinct chimera. The protocol developed to fold the largest chimera is described in detail below:

The homodimer domain 7 structure was assembled to the signaling domain by first engineering the linker between the TM and D7 domains of the first (N-terminal) monomer using the domain assembly protocol. Low energy models were then clustered and selected to build the region connecting the TM and D7 of the second (C-terminal) monomer using loop modeling. The lowest energy D7-TM-CT dimer structure was selected to assemble the remainder of the receptor.

Selected models of monomeric D6, dimeric D5,monomeric D4, dimeric VEGF bound D2-3, and monomeric D1 domains were then assembled to the D7-TM-CT dimer structure to generate a partial receptor dimer model (FIG. 5 step 1). The rationale for this intermediate assembly step was to build a starting scaffold incorporating all known dimeric domain interfaces and enabling sufficient conformational flexibility to assemble the domains 4 and 6, for which the binding interface in the dimeric receptor remains unknown. To account for the missing domains 4 and 6 and enable enough conformational diversity for the final insertion of these domains, distance constraints with significant tolerance were applied during the assembly. These distance constraints are defined by: (a) missing domains, based on the Euclidean distance between the N- and C-termini, and (b) missing linkers (Choi Y, et al. PeerJ. 2013 Feb. 12; 1:e1). All structures with significant violation of the distance constraints were filtered out. The remaining structures were clustered, and the best-scoring structures of the 20 largest clusters were used as input conformations for the final assembly step.

In the following step, the missing domains 4 and 6 were added (FIG. 5 step 2). Domain 4 was attached to the end of domain 3 in monomer A, and domain 6 to the start of domain 7 in monomer B. Two sets of constraints for the linkers were defined between domains 4 and 5, and between domains 5 and 6, as the assembly protocol attaches domains at the termini of a polypeptide chain instead of inserting them between 2 chains. All structures with significant violation of the distance constraints were filtered out, and the remaining structures were used as input templates for building the missing linkers and domains (FIG. 5 steps 3 & 4). The output structures were clustered, and the best-scoring model of each family were relaxed.

Since the other receptor chimera involved different subsets of domains, the assembly protocol was adapted to each specific topology, as described in FIG. 6.

### 4. Modeling and Assembly of CSF1R-Based Chimeras

Since CSF1R and c-MPL function as ligand-bound dimers, the chimera was modeled in a dimeric form. Three possible constructs for CSF1R-based chimeras were explored. For CSF1R, all three feature D4-5 of CSF1R and the active TM of c-MPL, the linker length between D5 and the TM defines the difference between them (CMR_{FL}: 27 residues, CMR_{INT}: 18 residues, CMR_{SHORT}: 10 residues), with both CMR_{FL} and CMR_{INT} including a predicted α-helical pseudo-domain owing to the linker length. A similar procedure as above was applied to assemble the chimeras. Further details on the preparation of the domain library are provided below.

### TM Domain:

The c-MPL TM active states modeled with EFDOCK-TM were used for the CSF1R constructs. The CSF1R constructs did not include the CT domain nor any pre-processed generation of a TM-EC complex.

Domains D4 and D5 were extracted from a deposited monomeric structure of CSF bound to CSF1R (4wrm). Missing loops were rebuilt to generate a complete monomeric structure of D4-5. D4 participates directly in forming the CSF1R dimer, but no dimeric active bound state of CSF1R is available. However, the D4-5 domains of CSF1R share 47% sequence similarity with SCFR D4-5, for which an active dimeric bound state structure has been determined (PDB code: 2e9w). Both receptors also share a conserved salt bridge known to stabilize the D4 dimer (Felix J, et al. Structure. 2015 Sep. 1; 23(9):1621-1631; Reshetnyak AV, et al. Mol Cell. 2015 Jan. 8; 57(1):191-201). Therefore, the D4-5 monomeric structure was docked, guided by the SCFR bound state. In the case of the SCFR constructs, the available bound state (2e9w) was used to rebuild missing loops to obtain a complete D1-5 dimeric active state. All models were idealized and relaxed in Rosetta to convergence prior to further modeling.

### Linker Domain: CSF1R:

CMR_{FL} and CMR_{INT} featured pseudo-domains predicted to be α-helical to obtain a reliable orientation of the flanking domains in the simulations and calculate mechanical couplings. These domains were predicted by inserting the sequence into both AlphaFold2 (Jumper, J., Evans, R., Pritzel, A. et al. Nature 596, 583-589 (2021)) and RoseTTAfold (Minkyung Baek et al. Science Aug. 19, 2021, Vol 373, Issue 6557, pp. 871476) given contextual information of the D4 domain and the TM monomeric sequence. The dimeric structure was also predicted with AF2 multimer (Richard Evans, et al bioRxiv 2021.10.04.463034), though this yielded a low-confidence unstructured model. For CMR_{FL}, AlphaFold2 returned a 16 residue α-helical motif flanked by 10 and 1 residues from the N- to C-termini direction and a 16 residue α-helical motif flanked by 3 and 1 residues for CMR_{INT}. The average pLDDT for both AlphaFold2 α-helix motifs was 83. While the RoseTTAfold returned a similar result, its self-confidence score was 26 and 21, respectively.

Further confirmation of these α-helical motifs was provided by running the sequence through three secondary structure prediction programs: PSIPRED (Daniel W A Buchan, et al., Nucleic Acids Research, Vol. 47, Issue W1, Jul. 2, 2019, W402-W407), predictprotein (Michael Bernhofer, et al., Nucleic Acids Research, Vol 49, Issue W1, Jul. 2, 2021, W535-W540), and jpred (Alexey Drozdetskiy, et al., Nucleic Acids Research, Vol 43, Issue W1, Jul. 1, 2015, W389-W394). All three returned with high confidence an α-helical region over the same span as the AlphaFold2 models. They differed in their confidence and prediction of the immediate flanking regions; for example, PSIPRED predicted, with low confidence, an additional 3 helical residues on the N-termini of the structural models. Since no consensus exists in these regions, the helical region was not extended. Thus, the pseudo-domain used for the assembly above is taken from the high-confidence AlphaFold2 model, with the respective linkers left to rebuild via the assembly protocol.

### Example 2

This example describes the materials and methods used in EXAMPLE 3 below.

### Cell Lines.

A549 (ATCC CCL-185) and HEK 293T/17(ATCC CRL-11268) cell lines were purchased from American Type Culture Collection (ATCC). Cell lines were maintained in complete DMEM or IMDM medium (Hyclone, Thermo Scientific, Gibco) with 10% fetal bovine serum (FBS) (Hyclone or Gibco), 1% glutamax (Gibco) and 1% penicillin/streptomycin (Gibco). A549.GFP-FFLuc cells were obtained from Dr. Stephen Gottschalk, Baylor College of Medicine, Houston, USA, and purity was verified by flow cytometry. A549.GFP-FFLuc VEGF-A¹⁶⁵ knock-out (VEGF-KO) cells were generated using CRISPR/Cas9. Guide RNAs were designed with the CRISPRscan algorithm (CRISPRscan.com), and four guide RNA sequences were selected to generate the KO cell line. Guide RNAs were synthesized by in vino transcription (NEB, HiScribe T7 High Yield RNA Synthesis Kit, E2040S). A total of 1 μg of four VEGF-A single-guide RNAs (sgRNA, 5′-GCCCCTGATGCGATGCGG-3′ (SEQ ID NO: 24), 5′-GAGCCGTGGTCCGCGCGG-3′ (SEQ ID NO: 25), 5′-CGGGCTCATGGACGGGTG-3′ (SEQ ID NO: 26), and 5′-GATGTTGGACTCCTCAGT-3′ (SEQ ID NO: 27)) were mixed with 1 μg recombinant Cas9 protein (CP01, PNA Bio) at room temperature and used to electroporate 0.25×10⁶ A549 cells (2 pulses of 1200V for 30 ms, Neon Transfection System, Invitrogen). Electroporated cells were expanded in antibiotic-free DMEM and 10% FBS. Single cell clones were generated by limiting dilution. Each clone was analyzed for VEGF-A¹⁶⁵ secretion by ELISA (R&D Systems, DY293B) and a clone with lowest VEGF-A¹⁶⁵ production was selected for the experiments. A549.GFP-FFLuc cells with overexpression of human VEGF-A¹⁶⁵ were generated by retroviral transduction with a vector encoding for human VEGF-A¹⁶⁵ and truncated human CD271 (DCD271) for selection. Transduced A549 cells were stained for CD271-PE (clone C40-1457, BD Biosciences) and CD271 positive cells were FACS sorted (SH800, Sony Biotechnology) to over 98% purity.

Peripheral Blood Mononuclear Cells from Heathy Donors.

Buffy coats were obtained from de-identified healthy volunteers at the Gulf Coast Regional Blood Center (Houston, TX, USA) or at the Center of Interregional Blood Transfusion SRK Bern (Bern, Switzerland).

### Generation of Retroviral Vectors and Supernatant.

To generate a retroviral vector for human VEGF-A¹⁶⁵ overexpression, human VEGF-A¹⁶⁵ cDNA (HG11066-G, Sino Biological) was cloned into the SFG retroviral vector backbone followed by an IRES sequence and a truncated human CD271 (ΔCD271) selectable marker gene (In-Fusion HD Cloning Kit, Clontech). To generate the ΔVEGFR2-AMPL receptor (VMR) constructs, the protein sequence of the human vascular endothelial growth factor receptor 2 (VEGFR2) was obtained from the Uniprot database (P35968.1). The natural signal peptide (SP) sequence was predicted using the Signal-IP4.1 server (http://www.cbs.dtu.dk/services/SignalP/) and replaced with a SP sequence derived from a human immunoglobulin as previously described. A myc-tag sequence (EQKLISEEDL; SEQ ID NO: 28)) was added at the VEGFR2 protein N-terminus for detection and selection. Codon optimization for expression in human cells and gene synthesis were performed by Geneart (Thermofisher). Three different VMR versions were generated by PCR cloning (In-Fusion HD Cloning Kit, Clontech) using the VEGFR2 and c-MPL plasmids as templates. The different chimeras were inserted into the SFG retroviral vector backbone. CAR constructs targeting the Ephrin Type A Receptor 2 (EphA2) antigen were obtained from Dr. Stephen Gottschalk, Baylor College of Medicine, Houston, USA, and their generation and functional validation were previously described. Transient retroviral supernatant for all constructs was prepared by transfection of 293T cells as described.

### Generation of Transgenic T Cells.

Peripheral blood mononuclear cells (PBMCs) were harvested from healthy donor buffy coats using density gradient centrifugation by Lymphoprep (Accurate Chemical and Scientific Corporation). Total PBMCs were activated in non-tissue culture treated 24-well plates (Corning) coated with OKT3 1 ug/ml (purified from hybridoma CRL-8001, ATCC or purchased from Biolegend) and anti-CD28 antibody 1 μg/ml (BD Biosciences or Biolegend), and IL2 (100 U/ml) or IL7 and IL15 (10 ng/mL each, Miltenyi Biotec) for 3 days, and transduced on retronectin (Takara Bio) coated non-tissue culture treated 24-well plates. After 48-72 hours, T cells were harvested and further expanded in T cell media (1:1 mixture of RPMI 1640 and Click's media, Hyclone, or RPMI 1640 alone) supplemented with 10% FBS, 1% penicillin-streptomycin, 1% glutamax, and IL2 (50 U/mL) or IL7 and IL15 (10 ng/mL each). To generate T cells transduced with both CAR and VMR vectors, activated PBMCs were first transduced with CAR supernatant for 24 hours, followed by a separate transduction with VMR supernatant for an additional 24-48 hours. After the second transduction, T cells were harvested and expanded for 7-10 days in fresh T cell media with IL2 (50 U/mL) or IL7 and IL15 (10 ng/mL each) and were fed every 3-4 days with fresh media and cytokines.

### Immunophenotyping.

To assess transduction efficiencies, T cells were stained with VEGFR2-PE (R&D Systems), CD19-APC and 7AAD (BD Biosciences). To assess T cell lineage and naïve/memory subset distributions, cells were stained with 7AAD, CD4-Krome Orange, CD8-Pacific Blue, CD45RA-APC, CD45RO-PE, CD62L-ECD and CCR7-V450 (BD Biosciences or Beckman Coulter). For analysis of STAT5 phosphorylation in response to VEGF, T cells were rested in cytokine free medium overnight and then stimulated with 25 ng/ml rhVEGF-A¹⁶⁵ (or indicated concentration) or IL15 10 ng/ml as positive control, for 1 hour. Cells were fixed with Cytofix (BD Biosciences), permeabilized with Perm Buffer III (BD Biosciences), washed thoroughly and stained with STAT5-PE (pY694) (clone 47/Stat5, BD Biosciences) for 1 hour.

Data acquisition was performed on a Beckman Coulter Gallios using Kaluza software, or on a FACSCanto or SORP-LSRII with BD FACSDiva software. Data analysis was performed with FlowJo software (Tree Star Inc.).

### Sequential Co-Culture Assay.

A549.GFP-FFluc cells were co-cultured with T cells in 6 replicates at an E:T ratio of 1:1 (75′000 cells each in a 48-well plate) in the presence or absence of 25 ng/ml rhVEGF-A⁶⁵. For cytokine analysis, culture supernatants were harvested 24 hours after initial plating or rechallenge of the co-culture. Tumor cells and T cells in co-culture were quantified every 3-4 days by flow cytometry. T cells were stained with CD3-APC (BD Biosciences), tumor cells were identified by GFP expression, and 7AAD negative live cells were quantified with counting beads (CountBright Beads, Life Technologies). Residual T cells were challenged with fresh A549.GFP-FFluc tumor cells (75′000 cells/well) in each replicate well when >90% of tumor cells were killed at the analysis timepoint, otherwise the killing was considered as incomplete and T cells were not rechallenged.

### Multiplex Cytokine Detection.

Co-culture supernatants were analyzed with the MILLIPLEX human CD8+ T-cell magnetic bead panel (EMD Millipore) and the Luminex 200 instrument (Luminex) to determine the concentration of human cytokines and cytotoxic granules in co-culture supernatants or in MSD mesoscale.

### Detection of VEGF-A¹⁶⁵.

Human VEGF-A¹⁶⁵ levels were analyzed in culture supernatant by ELISA (Human VEGF Duoset ELISA Kit DY293B, R&D Systems) or MSD Mesoscale (U-Plex human VEGF-A assay kit), and in mouse serum and lung tumor tissue lysates by MSD Mesoscale. To determine VEGF production in culture, A549 cells were plated in 48-well plates at a density of 2×10⁵ cells/well/ml. Culture supernatants were harvested 6, 24, and 48 hours later to quantify VEGF production. Mouse serum samples were analyzed at a 1:5 dilution. Protein concentration of 50 mg tissue lysate was determined with the Pierce BCA Protein assay Kit (ThermoFisher Scientific). Data were analyzed on the Mesoscale Discovery software and graphed with GraphPad Prism.

Gene Expression by Nanostring nCounter.

Activated PBMCs from 5 healthy donors were transduced with 41BBz IRES-CAR alone or double transduced with 41BBz IRES-CAR and VMR. Non-transduced T cells from each donor were expanded as controls. Transgenic T cells were purified by FACS or magnetic bead sorting. 41BBz IRES-CAR+VMR+ T cells were stained with VEGFR2-PE and CD19-APC, and double positive T cells were FACS sorted (SH800, Sony Biotechnologies) to >94% purity. 41BBz IRES-CAR+ T cells were selected with CD19 magnetic microbeads (Miltenyi Biotec) to >95% purity. Sorted T cells were co-cultured with A549.GFP-FFLuc-VEGF KO cells (E:T ratio 1:1) in 24-well plates in the presence or absence of rhVEGF-A¹⁶⁵ (25 ng/ml). After 3-4 days, live T cells and tumor cells were quantified by FACS. In cultures with complete tumor cell killing, live T cells were purified by Dead Cell removal Kit (Miltenyi Biotec), while T cells from cultures with residual tumor cells (>1%) were purified with a mixture of CD4 and CD8 magnetic beads and positive selection on LS columns (Miltenyi Biotec). From each sample, RNA was purified with the RNeasy Mini Kit (Qiagen) according to the manufacturer's instructions. Gene expression from a total of 36 RNA samples, from 9 experimental groups and four individual donors each were analyzed with the Immunology V2 Panel (NanoString). RNA quality control, Nanostring nCounter sample processing and data analysis were performed at the Genomic Technology Facility at the Center for Integrative Genomics at the University of Lausanne, Switzerland. Data normalization was performed in nSolver Analysis Software 4.0, using total counts per lane as normalization factor. Data were log 2 transformed, and hierarchical clustering and principal component plots were generated in R (version 3.4.4). Differential gene expression analysis was performed using the R Bioconductor package limma, using the limma trend approach. For pairwise comparisons of experimental conditions, moderated t-tests with paired samples were used, taking into account the sample donor. For each comparison, a separate linear model was fitted to the sub-set of data belonging to the two conditions. P-values were adjusted for multiple testing correction by the Benjamini-Hochberg method, separately for each comparison.

### Mouse Xenograft Experiments.

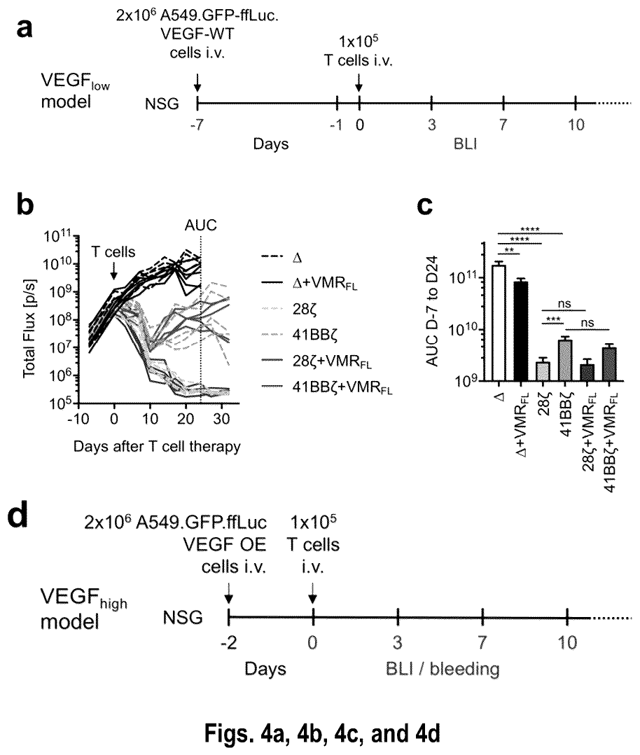

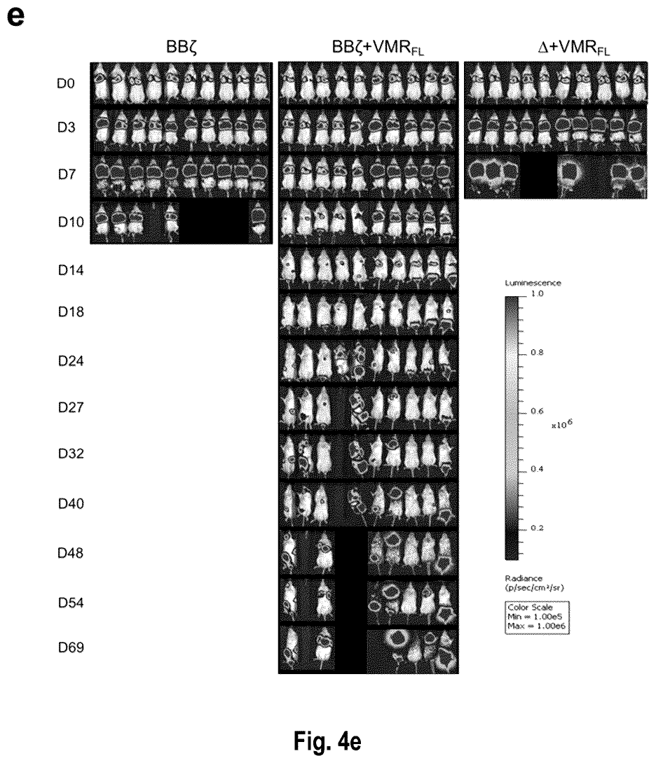

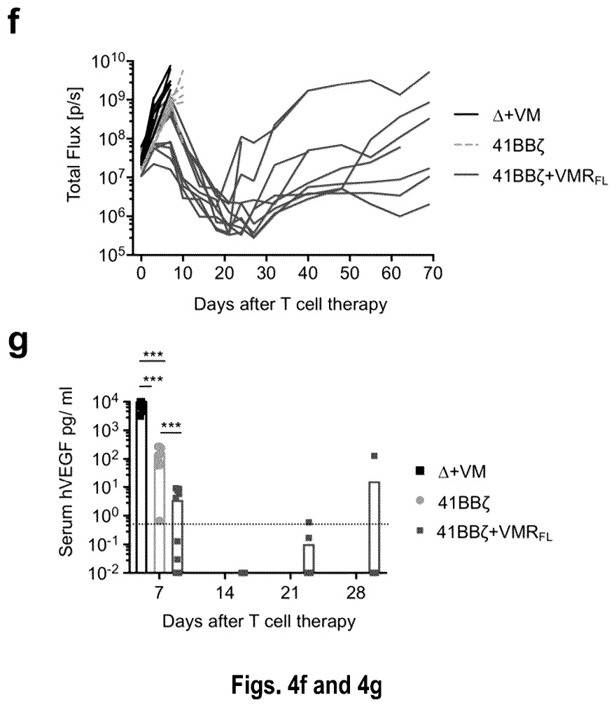

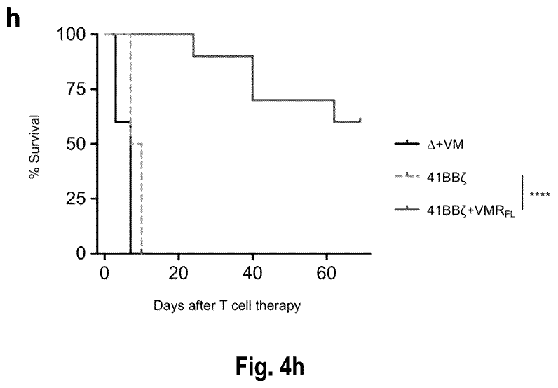

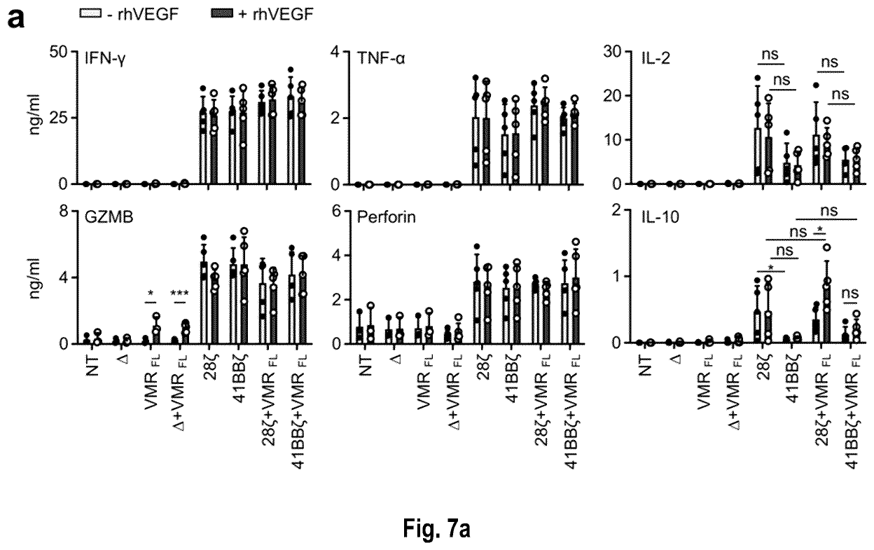

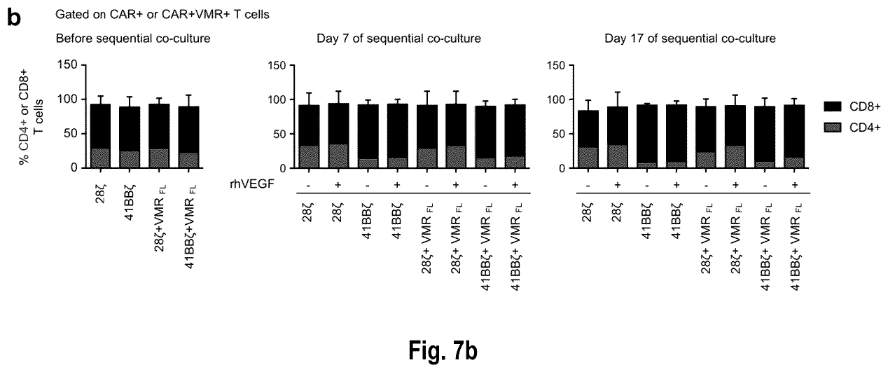

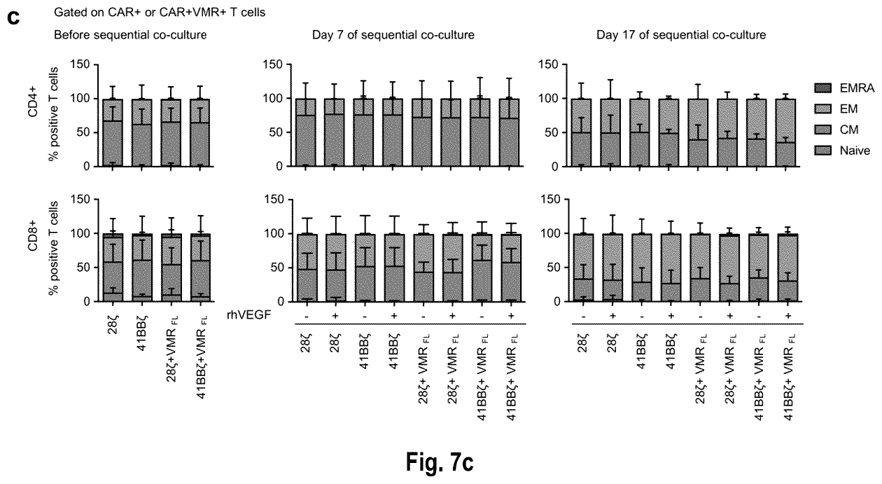

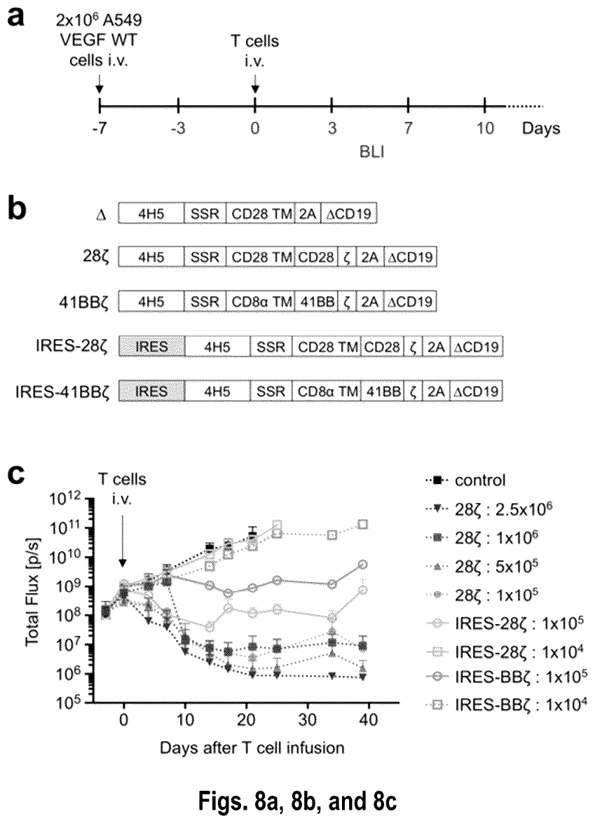

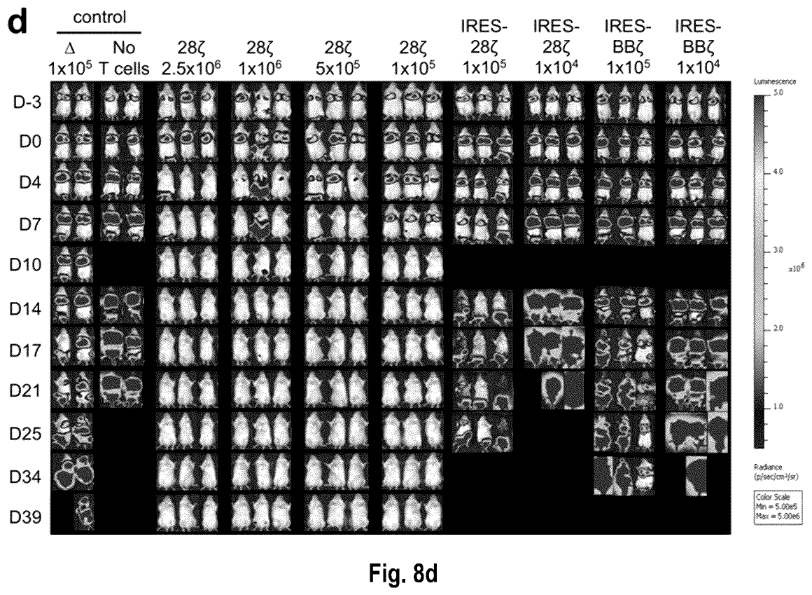

All animal studies were conducted in accordance with a protocol approved by Institutional Animal Care and Use Committee (IACUC) of Baylor College of Medicine. Female NOD-SCID-γc^{-/-} (NSG) mice (4-6 weeks old) were purchased from the Jackson Laboratory and housed at the Baylor College of Medicine Animal Facility. Animals were 6-8 weeks old at the start of the experiments. They were infused intravenously (tail vein) with 2×10⁶ A549.GFP-FFluc or A549.GFP-FFluc-VEGF-A¹⁶⁵ cells/mouse. Tumor burden was monitored once or twice weekly by bioluminescent imaging (BLI) using the Xenogen in vivo imaging system (IVIS) (Caliper Life Sciences). Transgenic T cells were intravenously (tail vein) infused at various doses (1×10⁴ to 2.5×10⁶/mouse) between 2-7 days after tumor challenge, depending on the model used (Day 7 for A549.GFP-FFLuc VEGF-A¹⁶⁵ WT (FIGS. 7 and 8), Day 2 for A549.GFP-FFLuc VEGF-A¹⁶⁵ OE (FIG. 4)). Mouse serum samples were collected for determination of hVEGF-A¹⁶⁵ levels at indicated timepoints. At the time of sacrifice, peripheral blood, serum, and organs were harvested for analysis. About 100 ul of blood was collected, let coagulate and serum was stored at −80° C. for further analysis. Lung tumor tissue fragments were collected in 1.5 ml Eppendorf tubes, snap-frozen in liquid nitrogen, and stored at −80° C. To prepare tissue lysates, frozen tumor fragments were crushed and dissolved in RIPA buffer (Cell Signaling, 9806S) containing a protease inhibitor cocktail (Roche, 78443). Lysates were incubated on ice for 30′, followed by centrifugation at 10′000×g for 10′ (4° C.) to remove extra tissue and debris. Lysates were stored at −80° C. for further analysis.

### Statistics.

Data were summarized using descriptive statistics. For continuous variables, comparisons were made by t-test. EC50 values were calculated upon log transformation of VEGF concentrations and normalization of dose response, followed by a non-linear fit. The area under the curve comparisons were analyzed with unpaired t-test and Welch's correction when appropriate. Survival of mice was analyzed by Kaplan Meier method, and significance was assessed with the log-rank test. Analyses were performed with GraphPad Prism version 8.1.2 or higher.

### Example 3

Engineered human adoptive T cell therapies for cancer are at the forefront of clinically applied synthetic biology. Direct cytotoxic targeting of tumor cells by adoptive transfer of autologous chimeric antigen receptor (CAR) T cells has changed clinical practice for patients with advanced B cell malignancies and multiple myeloma. In solid tumors, however, significant roadblocks limit broader success. To achieve sustained anti-tumor responses in vivo, T cells must recognize the tumor cells and receive distinct co-stimulation and cytokine signals from the environment. In most tumors, however, co-stimulation is dominated by co-inhibition, and cytokine signals required for sustained T cell expansion and memory formation are lacking or are overruled by immunosuppressive signals.

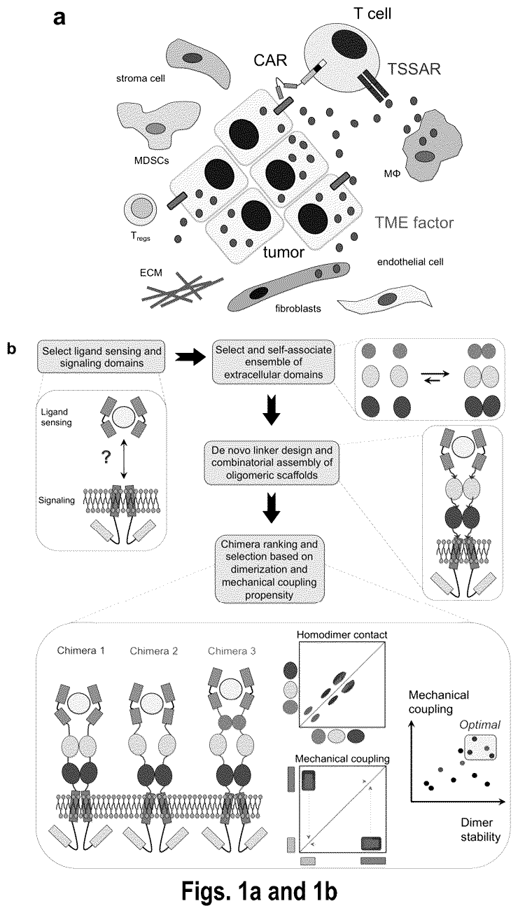

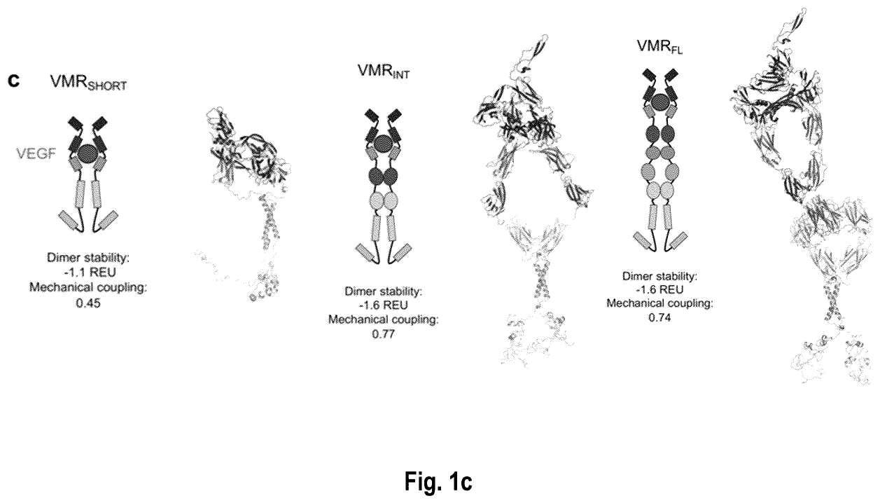

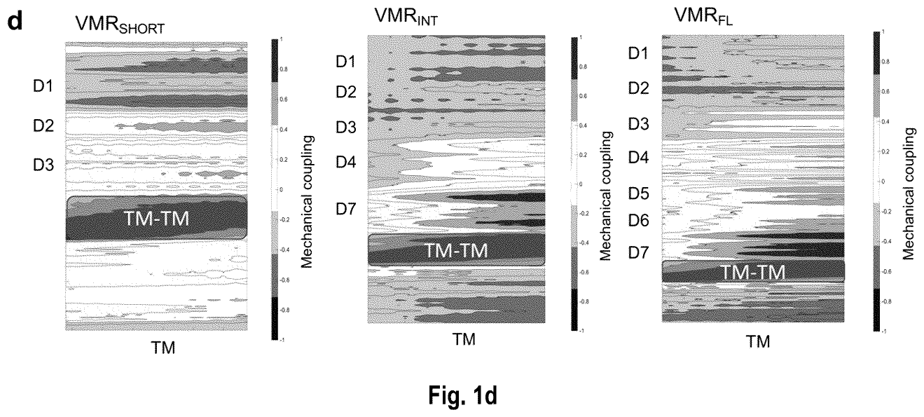

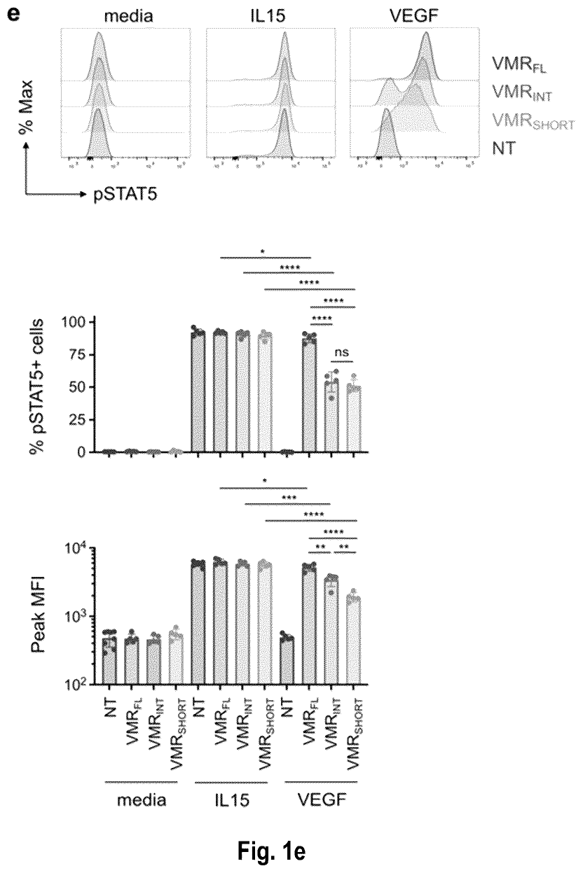

To address this challenge, a new class of allosteric biosensors called TME sensing receptors for sustained antitumor responses (TSSAR) were developed (FIG. 1a). TSSARs are designed to respond to soluble factors selectively abundant in TMEs and rewire ligand-induced signals to stimulate human T cells. It was hypothesized that building TME responsive advanced logic control with TSSARs can enhance both the potency and specificity of therapeutic T cells.

The design of TSSARs requires the wiring of specific pathways to arbitrary and unrelated extracellular cues. So far, receptors responding to soluble factors for human T cell therapy have been limited to either chimera assembled from related cytokine receptors or conventional CARs devoid of logic control. These chimeras were built using empirical approaches that lack generalizable engineering principles. While single pass receptor signaling usually involves receptor oligomerization, the underlying molecular mechanisms are often specific to each receptor family and can range from monomer to oligomer transition to structural rearrangement of pre-existing oligomers involving a wide range of protein motions. Receptor extracellular regions often consist of multiple domain repeats that interact and link the ligand binding to the signaling regions. How the precise number and association of the repeats modulates signal transduction remains poorly understood and has prevented the rational and efficient design of fully customized and optimized biosensor scaffolds to date. Accordingly, this disclosure provides a novel computational-experimental strategy for the de novo design and functional validation of TSSARs.

### Design and Selection of a Tumor Microenvironment Responsive Biosensor

The computational platform described here assembles de novo chimeric receptors from unrelated protein domains to create oligomeric structures that strongly signal upon sensing an orthogonal ligand that is abundant in the TME and involved in maintaining or promoting tumor fitness (FIG. 1b). This computational method is based on the direct optimization of features, such as dimerization propensity and long-range dynamic coupling that are strong predictors of signal transduction propensity (see Example 1). Therefore, the computational method can efficiently search through a diverse combination of domains, interactions, and inter-domain linker structures to rationally design functional receptor chimeric scaffolds. Through this approach, custom-built synthetic biosensors can be designed that can link binding of a user-defined chemical input signal to modular cellular responses through genetically encoded fusions of selected domains from diverse natural receptors.

The computational strategy may proceed in the following main steps (Example 1; FIG. 1b): (i) selection of the extracellular ligand-binding and intracellular signaling domains that define the desired input and output signals as well as the dimerizing extracellular and transmembrane (TM) domains for linking the ligand binding and signaling regions; (ii) self-association of individual domains into specific oligomers through docking; (iii) de novo inter-domain linker design and assembly of multi-domain oligomeric scaffolds; and (iv) ranking of receptor scaffold structures based on their propensity for dimerization and long-range mechanical dynamic coupling between ligand binding and signaling domains that are calculated using the Rosetta force field and Elastic Network models for investigating collective protein motions, respectively.

Vascular endothelial growth factor (VEGF) was selected as one exemplary input signal because it is highly abundant in TMEs and critical for tumor growth and metastasis in many cancers. c-MPL signaling was chosen as one exemplary output signal because it activates costimulatory, cytokine, and type I interferon pathways in T cells and produces enhanced anti-tumor function and memory T cell persistence when co-expressed with a tumor-targeted T cell receptor (TCR). Since it was an aim to design robust VEGF-MPL receptor (VMR) scaffolds that strongly signal through c-MPL upon VEGF sensing, the topology and individual domain structure of the native VEGFR2 and c-MPL receptors were first analyzed. While the structures of the full-length receptors remain elusive, several domain structures have been characterized (Yang, Y., et al. Proc Natl Acad Sci USA 107, 1906-1911 (2010); Leppanen, V. M. et al. Proc Natl Acad Sci USA 110, 12960-12965 (2013)). The VEGFR2 domains D2 and D3 strongly bound to VEGF and were selected as the input signaling region of the VMR. On the output signaling side, the structure and activation mechanism of cytokine receptor homologs of c-MPL indicate that strong ligand regulation and potent JAK/STAT signaling is achieved through the intricate coupling between the cytokine TM, juxtamembrane (JM) and the cytoplasmic (CT) regions. Hence, it was reasoned that an optimal TSSAR scaffold should couple the native TM region of c-MPL and not that of VEGFR2 to the c-MPL cytoplasmic domain. In the absence of structural information, the c-MPL TM domain in a dimer active signaling state was modeled from its sequence using EFDOCK-TM and then assembled into the entire signaling (TM+JM+CT) region using the assembly approach (Example 1).

Next, the extracellular (EC) linker regions were designed to connect the input to the output signaling parts. First, whether that region could be readily engineered by combining individual domains from native receptors was assessed. A library of all known native VEGFR and c-MPL extracellular (EC) domain structures were built and analyzed. Unlike c-MPL, all seven IgG-like VEGFR2 EC domains (D1-7) have been structurally characterized, and the isolated D4, D5, and D7 domains are known to homodimerize. A diverse set of chimeras was created to stringently test the ability of the disclosed computational approach in the rational design of full-length receptor scaffold structures with fine-tuned signal transduction propensity. The computational approach searched for combinations of VEGFR EC domains and connecting linkers that sample different density of contacts across the receptor dimerization interface and encode distinct levels of mechanical coupling, thereby modulating the signal transmission triggered by VEGF binding (Example 1; FIG. 1b). Three designed chimeras de novo assembled with different combinations of the D4 to D7 domains promoting distinct levels of ligand-induced homodimerization were selected for experimental validation (Example 1; FIGS. 1c, 5, and 6): VMR_{SHORT} with D1-3 linked to the TM; VMR_{INT} with D1-4+D7 linked to the TM; and VMR_{FL} with D1-7 linked to the TM. The calculations predicted that VMR_{SHORT} has a significant propensity to fold as a dimer and transmit signals to the TM domain, while VMR_{INT} and VMR_{FL} has stronger dimerization and signal transduction properties and provide superior VMR chimeric scaffolds for redirecting VEGF sensing into potent c-MPL signaling (FIGS. 1c and 1d; Example 1).

To experimentally validate the orthogonal signaling activity of the three designed VMR variants, VEGF-dependent STAT5 phosphorylation was assessed as a surrogate for c-MPL signaling in human T cells transduced with VMR_{SHORT}, VMR_{INT}, VMR_{FL}, or non-transduced (NT) control T cells. It was found that all three variants were capable of transmitting signals upon VEGF exposure, but to various degrees. VMR_{FL} produced the most efficient c-MPL pathway activation followed by VMR_{INT} and VMR_{SHORT} that were characterized by significantly lower levels of % pSTAT5+ cells and lower peak intensity of pSTAT5 staining when compared to VMR_{FL} or IL15 positive control (FIG. 1e). Importantly, no spontaneous pathway activation was detected in any of the three variants, indicating that the VMRs are fully ligand inducible. These results are consistent with the design calculations and indicate that the strength of dimeric contacts and inter-domain mechanical coupling are important determinants of signal transduction. Since VMR_{FL} displayed the highest ligand-induced signaling activity, VMR_{FL} was selected for functional evaluation in combination with CARs.

### Environment-Dependent Consequences of VMR_{FL} Activation in Human CAR T Cells

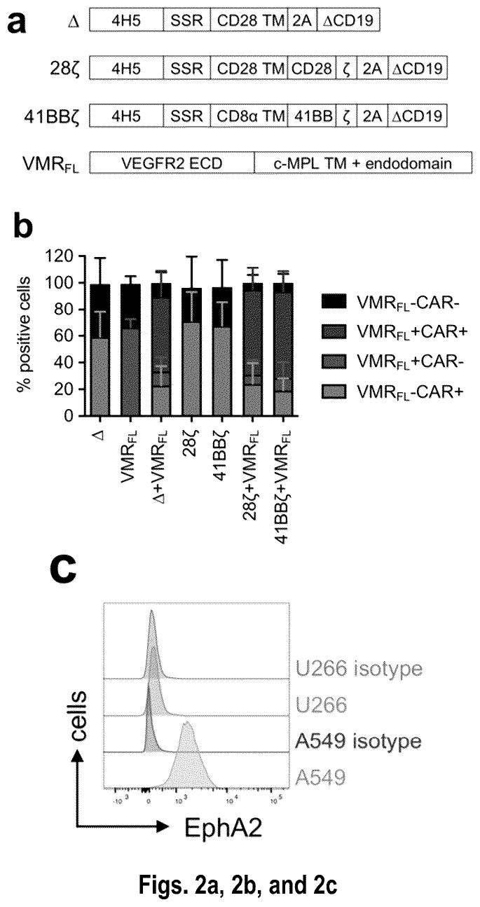

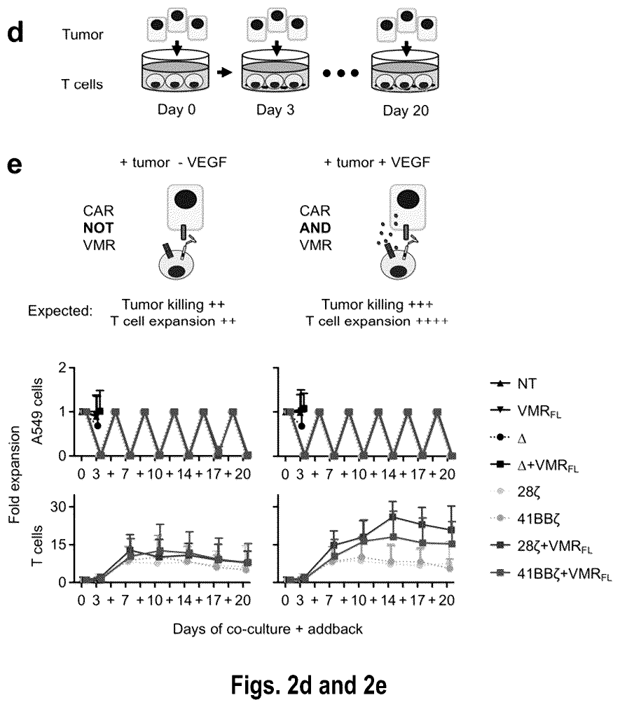

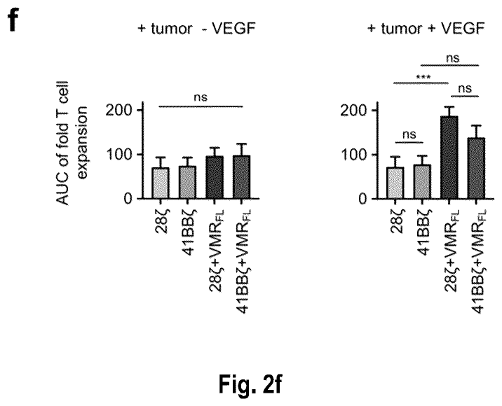

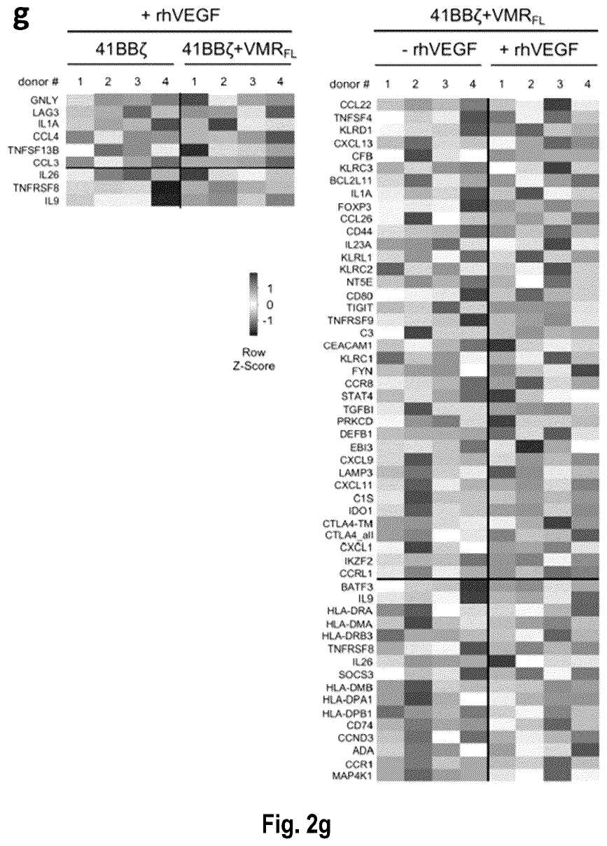

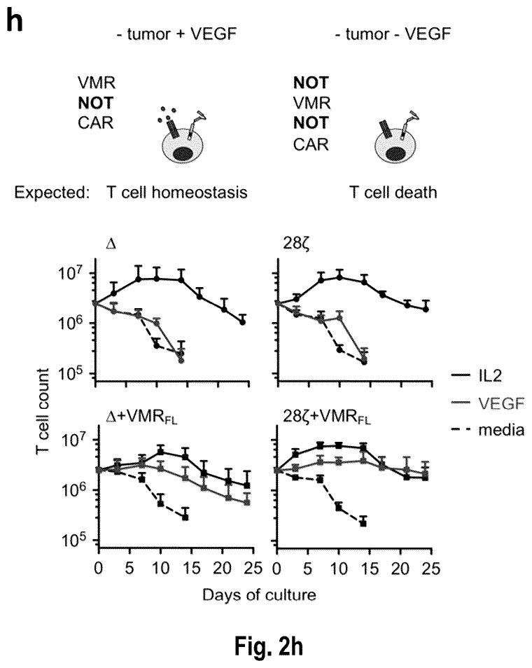

VMR_{FL} was co-expressed efficiently in activated human T cells with conventional second generation (28C, 41BBC) or non-signaling control (A) CARs targeting the antigen Ephrin A2 (EphA2) expressed on solid tumors (FIGS. 2a-c). The 41 single-chain variable fragment recognizes a conformational epitope that is exposed on a wide variety of malignant but not on normal cells, including A549 lung cancer cells (FIG. 2c). The impact of VMR_{FL} activation on tumor killing and T cell expansion was assessed in sequential co-cultures (FIG. 2d). As CAR and VMR_{FL} were expressed in a Boolean AND gate logic, full T cell activation with target killing and sustained T cell expansion only occurred in the presence of both tumor and VEGF (FIGS. 2e and 2f). While cytotoxicity was sustained in vitro even in T cells transduced with CAR alone, T cell expansion was significantly enhanced in the presence of VEGF and VMR_{FL} signaling. Co-expression of VMR_{FL} did not alter cytokine nor cytolytic granule production when compared to CAR alone and did not impact the T cell subset composition and differentiation status of T cells (FIG. 7). In the absence of tumor, VMR_{FL} activation by rhVEGF provided T cell homeostasis and survival similar to IL2, but at overall significantly lower levels (FIGS. 2g and 2h).

### Prediction of VMR_{FL} Engineered T Cell Response to Clinically Relevant VEGF Levels

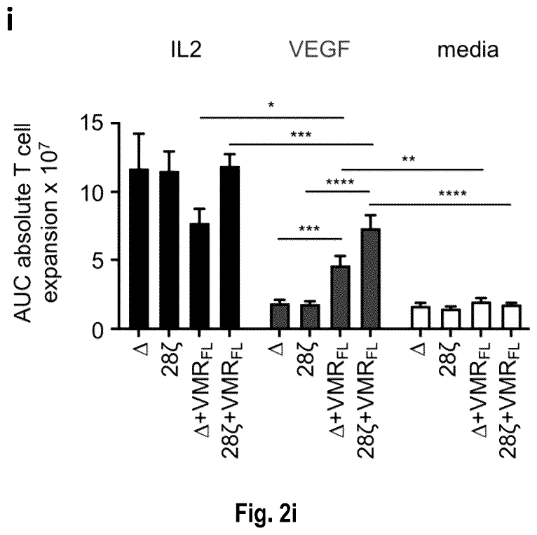

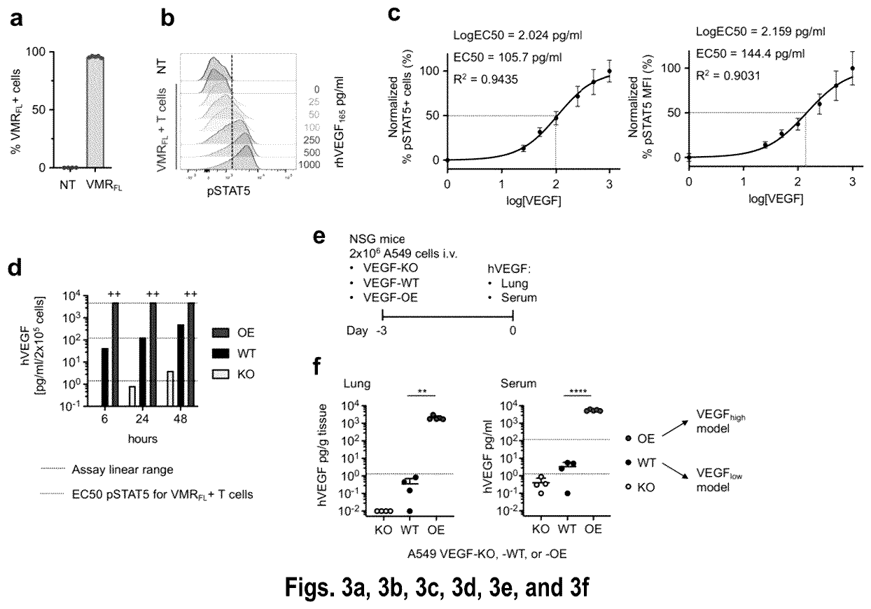

Serum VEGF levels have been intensively studied across tumor histologies and are significantly higher in cancer patients than in healthy individuals (Kut, C., et al. Br J Cancer 97, 978-985 (2007)). Body compartment distribution is altered in cancer, indicating that the biosensor will likely encounter higher VEGF levels in malignant than in normal tissue and enhance the specificity of the engineered T cells. The minimal VEGF concentration required to produce STAT5 phosphorylation in more than 50% of VMR_{FL}+ T cells was identified (FIGS. 3a-c), and the EC50 (% pSTAT5+ cells: EC50=106 pg/ml, pSTAT5 MFI: EC50=144 μg/ml) was determined (FIG. 3c).

To model the various VEGF levels in the mouse xenograft, A549 cell lines that produce different levels of human VEGF were generated, with wild-type (WT) and overexpression (OE) cell lines reaching levels above the VMR_{FL} activation threshold within 48 and 6 hours, respectively (FIG. 3d). VEGF_{low} (A549-WT) and VEGF_{high} (A549-OE) mouse xenograft models were also established (FIGS. 3e-f). In lung tissue lysates of VEGF_{high} mice, hVEGF levels reached a median of 1.775 ng/g protein (range 1.725-3.061, n=5), about 126-fold lower than the levels reported in lung cancer patients (median 224 ng/g protein, range 30-1870, n=71). In mouse serum, hVEGF reached concentrations comparable to those in cancer patients. Thus, the VEGF_{high} model is appropriate to evaluate VMR_{FL}+CAR T cell function in vivo.

### In Vivo Potency and Selectivity of VMR_{FL}+CAR T Cells in Metastatic Lung Cancer

Next, the in vivo impact and VEGF-dependence of VMR_{FL} function when combined with a CAR was evaluated. 1×10⁵ cells were determined as the minimal CAR T cell dose required for reaching a partial anti-tumor response in NSG mice engrafted with A549-VEGF-WT cells using IRES attenuated CAR constructs (FIG. 8). For the VEGF_{low} model, NSG mice were engrafted with A549.GFP-FFluc.VEGF-WT for 7 days before T cell therapy (FIG. 4a). Anti-tumor responses were quantified by bioluminescent imaging (BLI) (FIGS. 4b-c). At these limiting T cell doses, 41BBz IRES-CAR T cells consistently produced a partial anti-tumor response, while 28z IRES-CAR T cell function was variable and, in certain experiments, completely eradicated the tumors. Overall, 28z IRES-CAR T cells performed significantly better than 41BBz IRES-CAR T cells (FIGS. 4b-c) and, as expected, co-expression of VMR_{FL} did not further enhance the anti-tumor function of both CARs at low VEGF levels.

For the VEGF_{high} model reaching tissue VEGF levels at the lower end of those reported in lung cancer patients, A549.GFP-FFluc.VEGF-OE cells were engrafted for 2 days (more aggressive than the VEGF_{low} model) and treated with 1×10⁵ T cells (FIG. 4d). Mice treated with control A or 41BBz IRES-CAR T cells rapidly progressed and succumbed to the tumor. In contrast, mice treated with VMR_{FL}+41BBz IRES-CAR T cells were able to produce a potent anti-tumor response as measured by a rapid decrease in the BLI signal intensity (FIGS. 4e-f) and a significant reduction in human VEGF levels in mouse serum (FIG. 4g). Next, differential gene expression was assessed to analyze global immune response signatures in T cells. Several highly differentially expressed genes that are associated with enhanced T cell co-stimulation (e.g., CD80, TNFRSF8, HLA class II molecules) or enhanced effector function (e.g., GNLY) in cells with VMR_{FL} stimulation were identified. VMR_{FL} activation also led to a reduction in expression of genes associated with T cell exhaustion (e.g., CTLA4, LAG3, TIGIT) or factors associated with immune suppression (e.g., reduced transcription of NT5E, TGFB1, increased transcription of ADA) (FIG. 4h). Most importantly, the overall survival of mice treated with VMR_{FL}+41BBz IRES-CAR T cells was significantly enhanced compared to those treated with T cells expressing the CAR alone (FIG. 4i). Thus, VMR_{FL}+41BBz CAR T cells provide VEGF-dependent potent in vivo anti-tumor activity in a systemic non-small cell lung cancer xenograft model. It was demonstrated with the disclosed approach that tumor-relevant VEGF levels can be used to enhance the potency and specificity of engineered T cell therapies.

### Discussion

As demonstrated in this example, VEGF enriched in TMEs can be exploited as an orthogonal chemical stimulus for activating a synthetic signaling system through a designed biosensor in engineered T cells. The disclosed approach overcomes the lack of local co-stimulation and cytokine signaling required to enhance the potency and anti-tumor activity of limiting numbers of CAR T cells in the TME. A computational method was developed for the bottom-up assembly and design of biosensors with desired signaling activity. It demonstrates that biosensors for applications in engineered T cell therapy can be assembled from modular protein domains.

Except for protein-based materials built from protein subunits that assemble into specific supramolecular architectures through non-covalent interactions, computational approaches have mostly focused on designing single domain structures and binding. Past decades have witnessed the development of a large variety of protein binders ranging from antibody variants to de novo synthetic domains that can be grafted into existing protein scaffolds for enhanced target recognition and regulation of receptor activities. However, the existing approaches do not address the challenge of designing long-range communication functionality into multi-domain scaffolds. This property is essential for the development of biosensors providing precise ligand control of cellular activities but has been neglected so far. By leveraging fast protein structure and dynamics calculations, the computational platform provides a practical and efficient solution to the optimization of protein association and long-range mechanical coupling that governs signal transduction in single pass membrane receptors. Based on general biophysical principles and state-of-the-art modeling techniques, the approach is not limited to a particular scaffold architecture or molecular mechanism and can be applied to design a wide range of biosensor functions.

Splitting the T cell activation signals into separately expressed transgenic receptors allows for context-dependent tuning of T cell activation and anti-tumor activity and simultaneously enhances the tumor specificity of engineered T cells. TME-derived ILA or transforming growth factor b (TGF-b) have been used to elicit an immune-stimulatory signal with domains from classical co-stimulation (41BB) or cytokine receptors (IL7Ra). An engineered autocrine feedback loop based on GM-CSF, produced from T cells upon antigen recognition by the CAR, with a signaling output linked to IL18, has recently been explored as a strategy to enhance anti-tumor responses of CAR T cells. However, the autocrine T cell enhancement does not increase tumor specificity. A potential on-target off-tumor CAR activation would also lead to autocrine feedback loop activation. With the biosensor targeting VEGF that is a universal growth factor common to many tumor types, the unrelated c-MPL endodomain and its function in T cells in a TME specific manner were employed. c-MPL can activate signaling pathways related to both co-stimulation and cytokine signaling in T cells, and it is complementary to classical T cell co-stimulatory endo-domains such as 41BB. The disclosed strategy provides for the design of biosensors for cell engineering as in silico modeling predicts synthetic receptor signaling potency, and functional evaluation can focus on the most highly ranking chimeric assemblies. Thus, this disclosure provides a novel tool that can be combined with any CAR- or TCR-based cell therapy for cancer, activating pathways that favor long-term persistence and function of engineered T cells.

In summary, this disclosure provides a novel approach to designing biosensors for application in engineered human T cell therapies for cancer. The study indicates that targeting TME soluble factors widely shared among different tumor types results in potent and tumor-selective enhancement of anti-tumor responses that significantly improve treatment outcomes.

### Example 4

This example describes the materials and methods used in EXAMPLE 5 below.

### Cell Lines

MM.1S and HEK 293T/17 cells were purchased from the American Type Culture Collection (ATCC). NCI-H929 cells were purchased from the German Cell Culture Collection (DSMZ). MM.1S cells were maintained in RPMI 1640 (Gibco) with 10% % fetal bovine serum (FBS) (Gibco). NCI-H929 were maintained in RPMI 1640 with 20% FBS, 1% sodium pyruvate (Gibco) and 50 μM p2-mercaptoethanol (Gibco). 293T cells were maintained in Dulbecco's modified Eagle medium (DMEM) (Gibco) supplemented with 10% FBS. All cell culture medias contained 1% penicillin-streptomycin (Gibco), and 1% L-glutamine (Bioconcept). The PG-13 retroviral producer cell line for the generation of retroviral particles encoding Green Fluorescent Protein (GFP) and firefly luciferase (ffluc) (GFP.ffluc) was kindly provided by Dr. Stephen Gottschalk, Baylor College of Medicine. MM.1S and NCI-H929 cells were retrovirally transduced with GFP.ffLuc and FACS sorted (FACSAria IIu, BD Biosciences) for >98% purity. For the generation of 02-Microglobulin knock-out (B2M-KO) MM.1S cells were transduced with a lentiviral vector containing Cas9 and a single-guide RNA targeting B2M. Cells were selectively expanded in puromycin containing media and FACS sorted for HLA-A,B,C negative expression with HLA-A,B,C-PE (Biolegend) to over 98% purity. MM.1S.GFP.ffluc.B2MKO and NCI-H929.GFP.ffluc cells with overexpression of human CSF1 were generated by retroviral transduction with a vector encoding for human CSF1 and truncated human CD271 (ΔCD271) for selection. Transduced cells NGFR were positively selected with a CD271 selection kit (EasySep) to over 98% purity.

Peripheral Blood Mononuclear Cells from Healthy Human Donors

Buffy coats from de-identified healthy human volunteer blood donors were obtained from the Center of Interregional Blood Transfusion SRK Bern (Bern, Switzerland).

### Generation of Retroviral Vectors and Supernatant.

To generate a retroviral vector for human CSF1 overexpression, human CSF1 cDNA (#86797, addgene) was cloned into the SFG retroviral vector backbone followed by an IRES sequence and a truncated human CD271 (ΔCD271) selectable marker gene (In-Fusion HD Cloning Kit, Clontech). To generate the CSF1R-MPL receptor (CMR) constructs, the extracellular domain of the human colony stimulating factor 1 receptor (CSF1R) (HG10161-M, Sino Biological) was cloned with the TM and intracellular domain of c-MPL sequence into the SFG retroviral vector backbone followed by an IRES sequence and a truncated human CD271 (ΔCD271) selectable marker gene (In-Fusion HD Cloning Kit, Clontech). The natural signal peptide (SP) sequence was replaced with a SP sequence derived from a human immunoglobulin as previously described. Three different CMR versions were generated by PCR cloning (In-Fusion HD Cloning Kit, Takara Bio) using the CSF1R and c-MPL plasmids as templates. The different chimeras were inserted into the SFG retroviral vector backbone. A published fully human heavy-chain only (FHVH33) CAR (Lam et al. (2020) Nat Commun; 11(1):283) or an inhouse generated CAR based on a proliferation inducing ligand (APRIL) (Camviel et al. (2022) JITC 10:e005091) was used. APRIL-CARs target myeloma antigens BCMA and TACI and are designed based on a truncated monomeric APRIL unit (m) and varying signaling domains including ζ (mζ), 4-1BBζ (mBBζ) and CD28ζ (28ζ), or non-signaling truncated control (mΔ). Combinations of CMR and different APRIL-CARs (mζ.CMR, mBBζ.CMR and m28ζ.CMR) or FHVH33-CAR (FHVH33.BBζ) were cloned into a retroviral vector as a polycistronic construct separated by a 2A sequence. Transient retroviral supernatant for all constructs was prepared by transfection of 293T cells as described.

### Generation of Transgenic T Cells.

Peripheral blood mononuclear cells (PBMCs) were harvested from healthy donor buffy coats using density gradient centrifugation with Lymphoprep (Serumwerk). Total PBMCs were activated in non-tissue culture treated 24-well plates (Corning) coated with OKT3 1 ug/mil (Biolegend) and anti-CD28 antibody 1 ug/ml (Biolegend) and IL7 and IL15 (10 ng/mL each) (Miltenyi Biotec) for 3 days, and transduced on retronectin (Takara Bio) coated non-tissue culture treated 24-well plates. After 72 hours, T cells were harvested and further expanded in T cell media (RPMI 1640 supplemented with 10% FBS, 1% penicillin-streptomycin, 1% glutamax, and IL7 and IL15 (10 ng/mL each).

### Immunophenotyping.

To assess transduction efficiencies, T cells were stained with CD 115-PE (Biolegend), CD271-APC and DAPI (Biolegend). For analysis of STAT5 phosphorylation in response to CSF1, T cells were rested in cytokine free medium overnight and then stimulated with 10 ng/ml CSF1 (or indicated concentration) or IL15 10 ng/ml as positive control, for 30 minutes. Cells were fixed with Cytofix (BD Biosciences), permeabilized with Perm Buffer III (BD Biosciences), washed thoroughly and stained with STAT5-PE (pY694) or STAT5-AF647 (pY694) (clone 47/Stat5, BD Biosciences) for 1 hour.

Data acquisition was performed on BD FACS LSR II, SORP or Fortessa (BD Biosciences) using DIVA software. Data analysis was performed with FlowJo software (Tree Star Inc.).

### Sequential Co-Culture Assay.

NCI-H929.GFP.ffluc and MM.1S.GFP.ffluc cells were co-cultured with T cells in 6 replicates at an E:T ratio of 1:10 (50′000 T cells and 500′000 target cells per well in a 48-well plate) in the presence or absence of 10 ng/ml CSF1. For cytokine analysis, culture supernatants were harvested 24 hours after initial plating of the co-culture. Tumor cells and T cells in co-culture were quantified every 3-4 days by flow cytometry. T cells were stained with CD3-BV711 (Biolegend), tumor cells were identified by GFP expression, and DAPI negative live cells were quantified with counting beads (CountBright Beads, Life Technologies). Residual T cells were challenged with fresh target cells (500′000 cells/well) in each replicate well when >60% of tumor cells were killed at the analysis timepoint, otherwise the killing was considered as incomplete and T cells were not rechallenged.

### Single- and Multiplex Cytokine Detection.

Co-culture supernatants were analyzed with the U-plex human CSF1 or CAR-T cell combo 1 kit (Mesoscale Discovery) and analyzed on a MESO QuickPlex SQ 120 (Mesoscale Discovery). Data were analyzed on the Mesoscale Discovery software and graphed with GraphPad Prism.

### Mouse Xenograft Experiments.

All animal studies were conducted in accordance with a protocol approved by the Veterinary Authority of the Swiss Canton of Vaud and performed in accordance with Swiss ethical guidelines. NOD-SCID-γc−/− (NSG) mice were bred and maintained at the animal facility of the University of Lausanne. Animals were 7-11 weeks old at the start of the experiments, both males and females were used, and experimental groups were randomized based on animal weight at start of experiment. For the NCI-H929 and NCI-H929_{CSF1} model, NSG mice were injected with 3×10⁶ NCI-H929.GFP.ffluc or 6×10⁶ NCI_{CSF1}.H929.GFP.ffluc cells intravenously (Lv.) on day 0. Adoptive T cell transfer was performed with 5×10⁶ T cells i.v. on day 10 in the NCI-H929 model and on day 10 or day 21 in the NCI-H929_{CSF1} model. For the MM.1S and MM.1S_{CSF1} model, NSG mice were injected with 3×10⁶ MM.1S.GFP.ffluc.B2MKO cells or MM.1S_{CSF1}GFP.ffluc.B2MKO_{CSF1} i.v. on day 0. Adoptive T cell transfer was performed with on day 3 in the MM.1S model and on day 10 in the MM.1S_{CSF1} model with 5×10⁶ APRIL-CAR or 0.25×10⁶ FHVH33-CAR T cells i.v. Tumor growth was monitored twice a week or weekly by bioluminescent imaging (BLI) on a Xenogen IVIS Lumina II instrument using Living Image software. Data analysis was performed on Living Image software. Survival was determined according to humane endpoints as defined in the score sheet of the approved protocol.

### Statistics.

Data were summarized using descriptive statistics. Areas under the curves (AUCs) were calculated for T cell expansion data and tested for significant using t-test with Welch's correction. Normality assumption was examined, and log transformation was performed if necessary to achieve normality. Comparison of mouse bioluminescence was calculated with multiple ANOVA test. Survival analysis was carried out using the Kaplan-Meier method. The Wilcoxon test was used to assess statistically significant differences between groups of mice. GraphPad Prism 9 software was used. P values <0.05 were considered statistically significant.

### Example 5

In multiple myeloma patients, high levels of tumor-associated macrophages (TAMs) have been associated with poor prognostic outcomes independent of disease stage or tumor burden. Colony stimulating factor 1 (CSF1) is a key cytokine for driving the development of tumor-associated macrophages that harbor an immunosuppressive M2-like phenotype. Elevated levels of serum CSF1 have been reported in a subset of multiple myeloma patients and are associated with poor prognosis in multiple myeloma as well as in several other types of cancer. Targeting the CSF1-CSF1 receptor (CSF1R) axis has been explored in many cancer types with clinically approved drugs. In mouse models of multiple myeloma, CSF1R blockade was able to delay multiple myeloma growth which highlights the importance of the CSF1R-CSF1 axis for multiple myeloma survival and progression. To exploit the increased CSF1 levels in multiple myeloma patients, a novel chimeric receptor that binds soluble CSF1 from the environment and provides signals for enhanced T cell survival and function through a c-MPL-derived endodomain was developed. C-MPL harbors the advantage of providing signaling that is reminiscent of T cell costimulation and cytokine signals and has been previously described as a viable strategy to enhance T cell persistence when coexpressed in TCR-transgenic T cells. The novel CSF1R-MPL-receptor (CMR) has the capacity to provide additional stimuli to engineered T cells and support sustained T cell function and persistence in the immunosuppressive BMME. In this example, how coexpression of CMR can enhance functional performance of CAR T cells targeting MM in vitro and in vivo was investigated.

### Development and Characterization of a CSF1R-cMPL Chimeric Receptor (CMR)

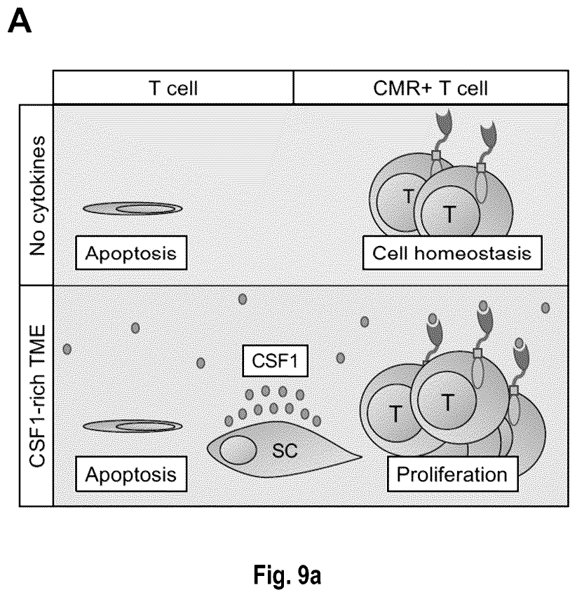

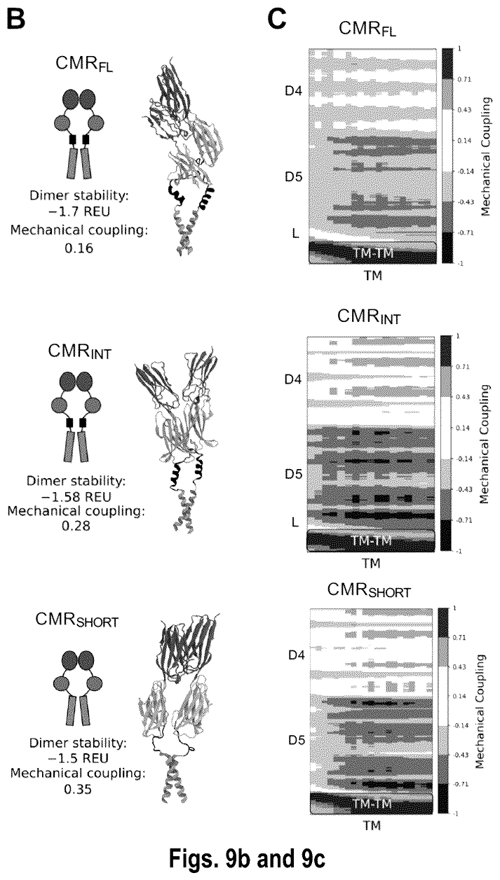

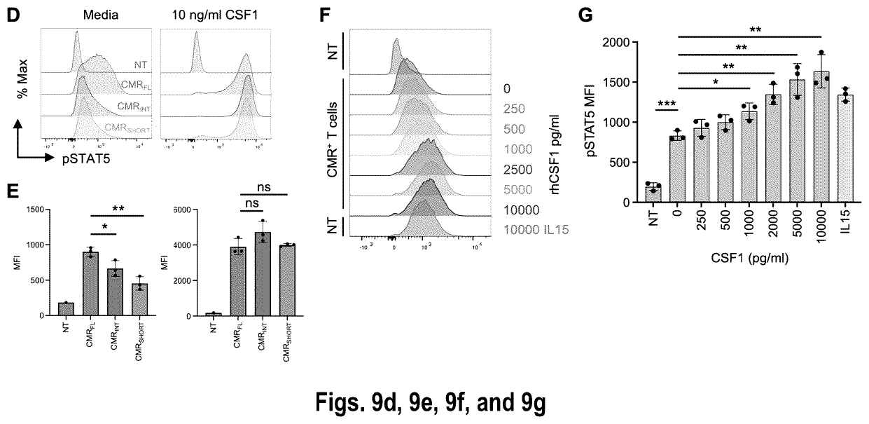

Using the computational pipeline, a chimeric receptor that binds soluble CSF1 in the tumor microenvironment via the CSF1R extracellular domain (ECD) and signals through the o-MPL transmembrane (TM) and intracellular domains (ICD) was developed. The CMR is designed for low-level baseline constitutive activity to enable T cell homeostasis in the absence of cytokines and a dose-dependent response to CSF1 for enhanced proliferation and persistence (FIG. 9a). As T cells naturally lack CSF1R, the CMR provides a selective orthogonal tumor microenvironment specific input to the engineered T cells, providing signals for T cell expansion and survival through c-MPL. Based on the predictive models, CMR variants with different lengths of linker sequence to modulate the level of mechanical coupling that determines ligand-induced responses and baseline receptor signaling was designed (FIGS. 9b-c). These variants include either the full-length CSF1R and c-MPL linker sequence (CMR_{FL}), a truncated CSF1R and c-MPL linker sequence (CMR_{INT}) or only the c-MPL linker sequence (CMR_{SHORT}). The predicted coupling was the highest for the CMR_{SHORT} construct. Therefore, the largest difference between constitutive and ligand-induced activities for that variant is expected.

To test the signaling capacity of the CMR variants, human T cells were transduced with CMR_{FL}, CMR_{INT}, and CMR_{SHORT} and measured phosphorylated STAT5 (pSTAT5) levels at baseline (media) and after exposure to 10 ng/ml of recombinant human CSF1 (rhCSF1) in cell culture. As predicted, CMR_{FL} provided intermediate constitutive pSTAT5 signaling in media only condition which was significantly reduced in CMR_{INT} (P=0.01) and CMR_{SHORT} (P=0.0026) (FIGS. 9d-e). All constructs provided similar maximum phosphorylated STAT5 (pSTAT5) upon addition of rhCSF1 while non-transduced (NT) T cells did not respond to rhCSF1. Consistent with the structure modeling predictions, the largest difference between constitutive and ligand-induced activities were measured for CMR_{SHORT}. To utilize the intermediate constitutive pSTAT5 levels CMR_{FL} (labelled as CMR) were chosen for all subsequent experiments. CMR+ T cells produced enhanced pSTAT5 signaling with increasing concentrations of CSF1 in a dose-dependent manner (FIGS. 9f-g). These data demonstrate that CMR is functional with baseline constitutive activity and responsiveness to CSF1 particularly in the clinically relevant range of >1000 pg/ml found in tumor microenvironments.

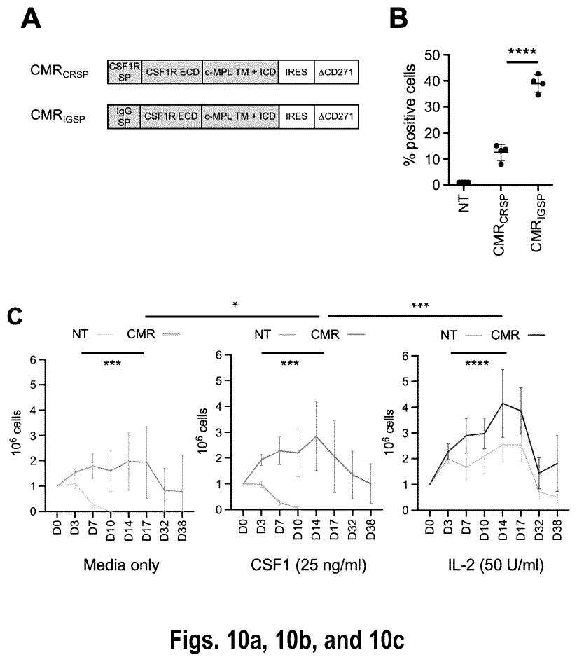

Next, it was sought to optimize the transduction efficiency of CMR in human T cells by exchanging the endogenous CSF1R signal peptide (CRSP) with the IgG signal peptide (IGSP)(FIG. 10a). Both constructs are flanked by an internal ribosomal entry site (IRES) and a truncated nerve growth factor receptor (ΔCD271) as a reporter of protein expression. Transduction efficiency of CMR_{IGSP} was superior to that of CMR_{CRSP} (39.00%±3.52 vs. 12.55±3.14%, P<0.000, FIG. 10b), and CMR_{IGSP} was used for all subsequent experiments and hereafter stated as CMR. To test whether the constitutive activity of CMR could enhance T cell survival and expansion in conditions devoid of cytokines, T cells were quantified over time in various conditions. In cytokine-free media, NT T cells did not expand and rapidly collapsed within 10 days of cytokine deprivation, while CMR+ T cells expanded and remained viable before collapsing 30 days after NT T cells (CMR_{media} vs. NT_{media}: P=0.0003, FIG. 10c). In media containing CSF1, NT T cells collapsed at an identical time point as in cytokine free media, confirming that non-engineered human T cells are not responsive to CSF1. On the contrary, CMR+ T cells exposed to CSF1 expanded significantly better than in cytokine-free media and at comparable levels as NT T cells cultured in IL-2, which is a well-known cytokine mediating T cell homeostasis and growth (CMR_{CSF1} vs. NT_{CSF1}: P=0.0001; CMR_{CSF1} vs. CMR_{media}: P=0.0135; CMR_{CSF1} vs. NT_{IL-2}: P=ns). Addition of IL-2 led to a further enhanced expansion of CMR+ T cells, indicating additional impact of IL-2 and the constitutive CMR signaling (CMR_{IL-2} vs. NT_{IL-2}: P<0.0001; CMR_{IL-2} vs. CMR_{CSF1}: P=0.0009). Taken together, the data demonstrate that transgenic CMR can greatly enhance in vitro survival of T cells and mediates baseline expansion in the absence of cytokines which is further enhanced in the presence of CSF1. Importantly, the constitutive CMR signaling did not provoke uncontrolled expansion of T cells in culture as these naturally contracted with a 30 days delay compared to NT T cells.

### Assessment of Serial Killing Capacity, Expansion, and Cytokine Secretion of CAR+CMR T Cells Compared to CAR T Cells

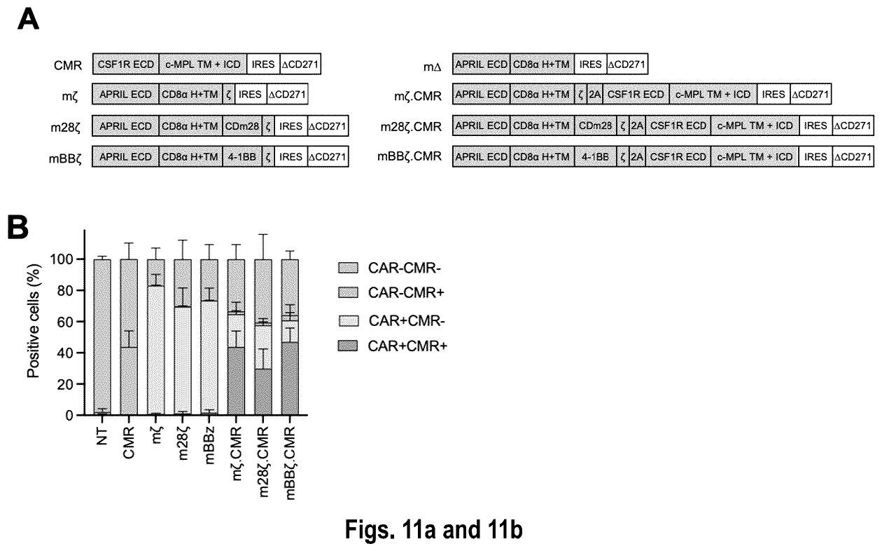

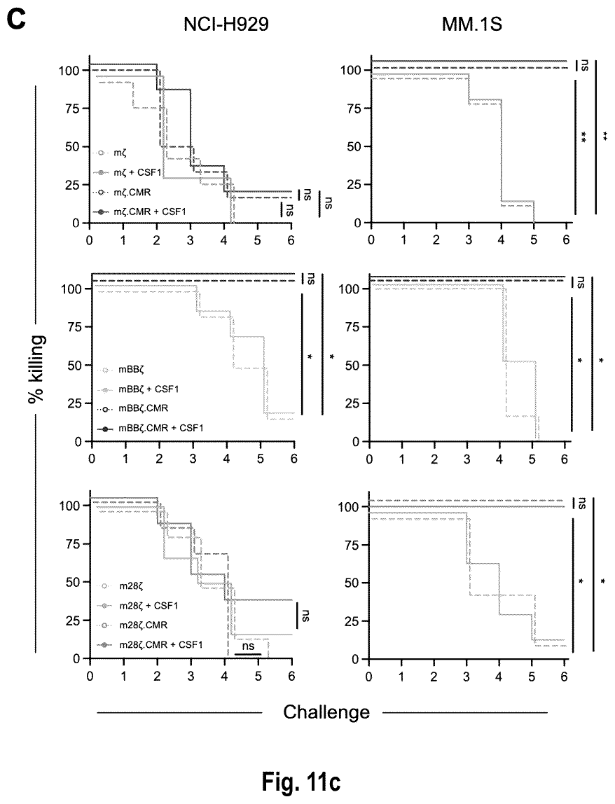

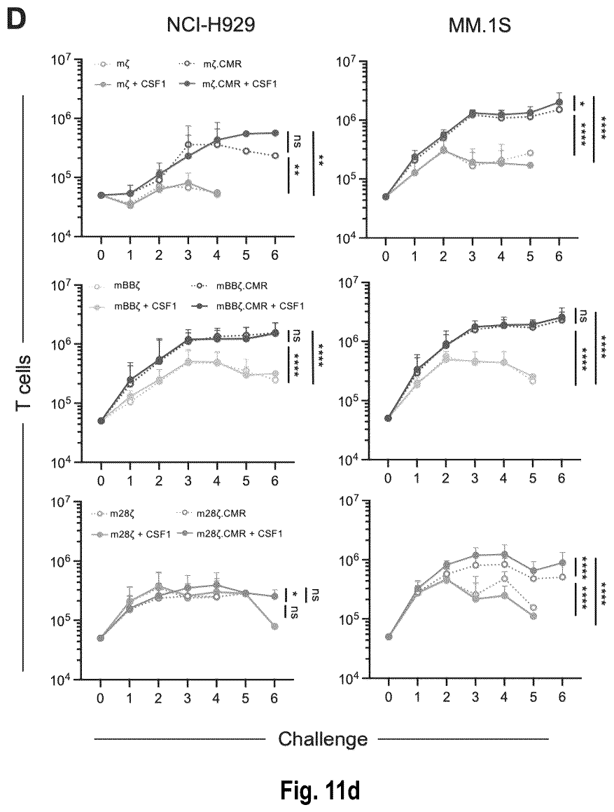

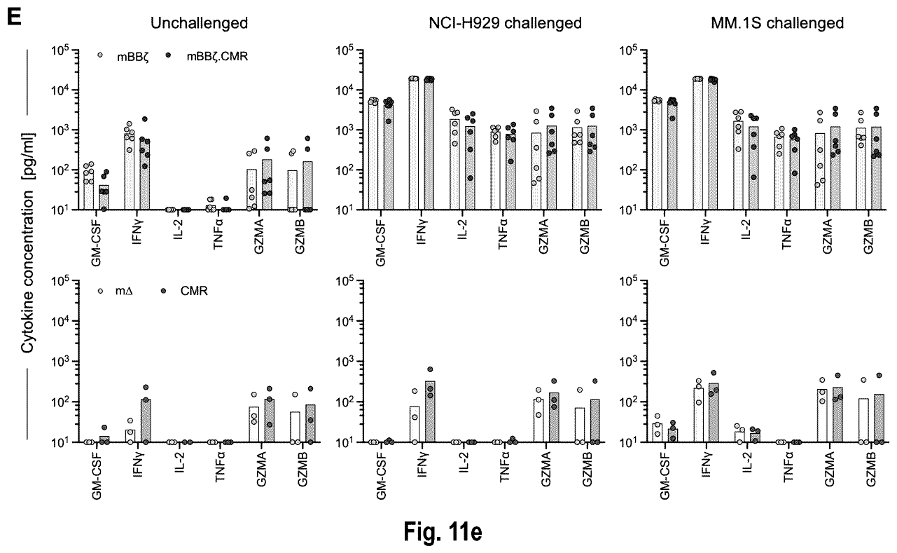

To determine the functionality of CMR in the context of adoptive cell therapy (ACT), CMR was co-expressed with CARs of varying signaling domains that target the myeloma antigens BCMA and TACI through their common ligand APRIL (APRIL-CARs) (Camviel N, et al. J Immunother Cancer. 2022; 10(11)). As CAR T cell function and phenotype can vary depending on the signaling and costimulatory endodomain used, it was sought to identify which CAR signaling domain would produce the best functional performance when combined with CMR signaling. Therefore, three CARs with varying endodomains in combination with CMR: A first-generation CAR (mζ) that does not contain built-in costimulation, and two second-generation CARs (m28ζ and mBBζ) that contain two different classes of costimulatory endodomains were tested. Combinations of CMR and different APRIL-CARs (mζ.CMR, mBBζ.CMR, and m28CMR) were encoded in single polycistronic retroviral vectors and separated by a 2A sequence (FIG. 11a). High transduction efficiencies were achieved for all constructs (FIG. 11b).

Next, whether coexpression of CMR and CAR in T cells can enhance the T cells' capacity to kill tumor cells was investigated under stress conditions by utilizing a sequential coculture assay. In brief, NCI-H929 or MM.1S myeloma cells were added in a 1:10 effector:target ratio every 3-4 days to wells containing either CAR+ or CAR+CMR T cells in cytokine-free media. To test the impact of CSF1 on CAR+CMR T cells, cells were cultured in media with 10 ng/ml CSF1 or media alone. In cytokine-free and CSF1-containing conditions, no significant difference was observed in serial killing between mζ vs. mζ.CMR and m28(vs. m28ζ.CMR T cells when challenged with NCI-H929 (FIG. 11c). However, in the mBBζ conditions, serial killing capacity of mBBζ.CMR T cells (mBBζ_{media} vs. mBBζ.CMR_{media} P<0.005) was significantly improved. When challenged with MM.1S cells in cytokine-free conditions, serial killing capacity was improved for all CAR T cells coexpressing CMR compared to CAR T cells without CMR (mζ_{media} vs. mζ.CMR_{media} P=0.0079, m28ζ_{media} vs. m28ζ.CMR_{media} P=0.035, mBBζ_{media} vs. mBBζ.CMR_{media} P=0.0005). As CAR+CMR T cells reached the maximum number of challenges provided by the assay in cytokine-free conditions, no further improvements were detected in serial killing through addition of rhCSF1. These results indicate that baseline signaling by CMR expression greatly enhances repeated killing capacity of all CAR T cells challenged with MM.1S and of mBBζ CAR T cells challenged with NCI-H929.

Next, whether CMR coexpression could enhance CAR T cell expansion during serial tumor challenge and whether expansion could be further improved by addition of CSF1 were investigated. For NCI-H929 challenged CAR T cells in media alone, CMR coexpression significantly enhanced expansion capability in mζ.CMR T cells (mζ_{media} vs mζ.CMR_{media}: P=0.0013) and mBBζ.CMR T cells (mBBζ_{media} vs. mBBζ.CMR_{media}: P<0.0001, FIG. 11d) but not in m28ζ.CMR T cells (m28ζ_{media} vs. of m28ζ.CMR_{media}: P=ns). Addition of CSF1 led to a minor improvement in expansion of m28ζ.CMR T cells but not mζ.CMR or mBBζ.CMR T cells (m28ζCMR_{media} vs. m28ζ.CMR_{CSF1}: P=0.026). In MM.1S challenged cultures, all CAR+CMR T cells expanded significantly better than the corresponding CAR T cell condition in the absence of CSF1 (mζ_{media} vs. of mζ.CMR_{media}: P=0.0001; mBBζ_{media} vs. mBBζ.CMR_{media}: P<0.0001; m28ζ_{media} vs. m28ζ.CMR_{media}: P<0.0001). Addition of CSF1 significantly enhanced the expansion of mζ.CMR (mζ.CMR_{media} vs. of mζ.CMR_{CSF1}: P=0.0185) and m28ζ.CMR T cells (m28ζ.CMR_{media} vs. m28ζCMR_{CSF1}: P<0.0001). Expansion of mBBζ.CMR T cells was the highest among all groups and was not further improved via CSF1. The data shows that CMR coexpression alone can greatly enhance the expansion capability of CAR T cells challenged with different myeloma cell lines. This improvement in T cell expansion could be further enhanced through addition of CSF1 in MM.1S challenged mζ.CMR and m28ζ.CMR cocultures. Among all constructs, mBBζ.CMR expanded the best in both NCI-H929 and MM.1S challenged conditions, indicating a synergistic potential of c-MPL and 41BB signaling.

Given that CMR coexpression can vastly enhance T cell expansion, particularly in combination with a BB CAR, whether cytokine production was affected after tumor challenge or in the absence of target antigen was analyzed. Cytokine levels of GMCSF, IFNγ, TNFα, IL-2 as well as granzyme A and B levels in coculture media were similar between mBBζ and mBBζ.CMR T cells challenged with NCI-H929 or MM.1S with no significant difference detected (FIG. 11e). High IL-2 concentrations were detected in both mBBζ and mBBζ.CMR conditions, indicating that CMR coexpressing CAR T cells could benefit from the synergistic signaling of IL-2 and CMR as previously observed (FIG. 9f). This observation could explain why addition of CSF1 did not improve expansion in some conditions, since IL-2 and CMR baseline signaling are superior to CSF1 mediated CMR signaling alone.

In unchallenged conditions, low amounts of IFNγ were detected in both mBBζ and mBBζ.CMR, indicating that low spontaneous baseline IFNγ production is driven by tonic signaling of the CAR rather than CMR. T cells that express a non-signaling CAR (mΔ) or CMR did not secrete notable amounts of cytokines or cytotoxic granules, highlighting that constitutive CMR signaling does not cause high constitutive cytokine secretion. In summary, no increase in cytokine secretion of mBBζ. CMR compared to mBBζ T cells during tumor challenge and very limited baseline cytokine secretion in the absence of target antigen were observed, indicating that cytokine secretion mostly depends on target antigen detection and signaling provided by the CAR.

### Determining the Impact of CMR Signaling on In Vivo Anti-Tumor Function of CAR T Cells in Myeloma Mouse Xenograft Models.

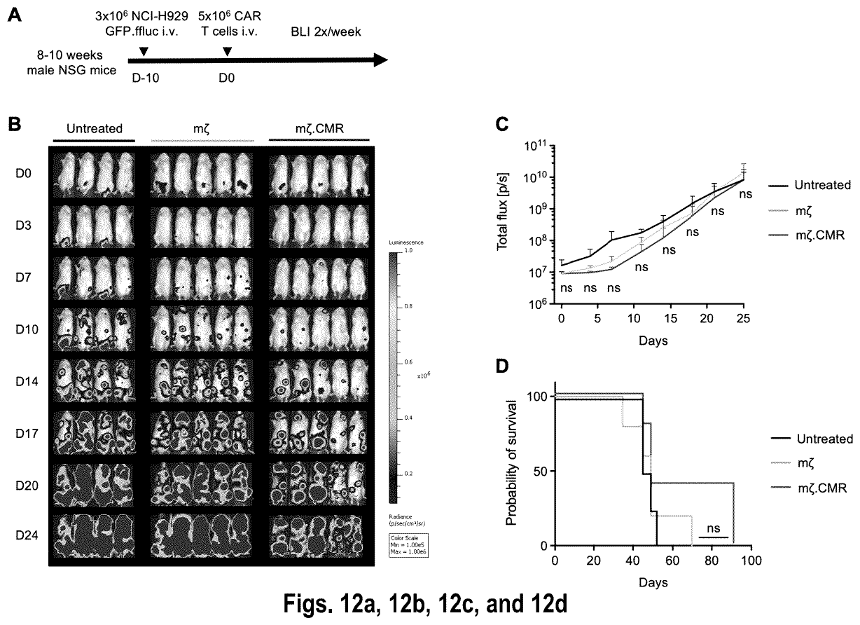

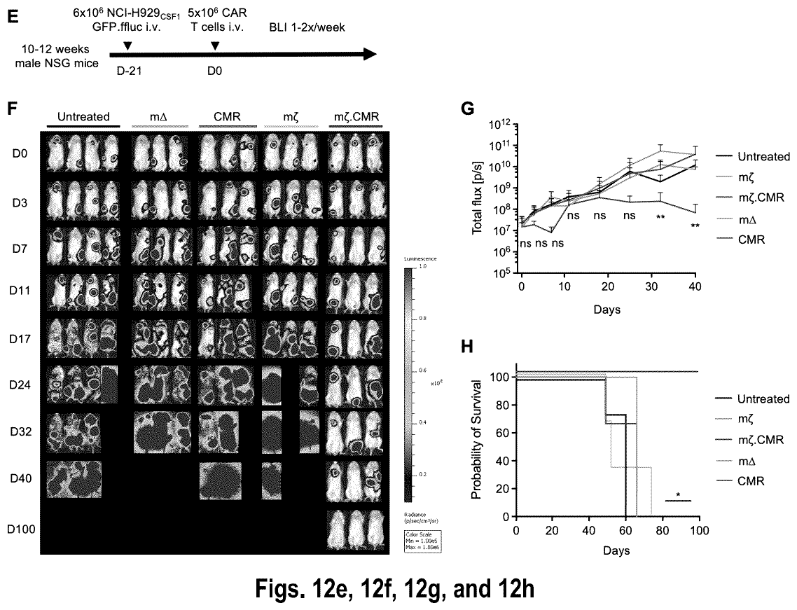

To investigate whether the improved in vino cytotoxic activity of CMR+CAR T cells translates into enhanced in vivo tumor control, adoptive cell transfer (ACT) experiments were performed in NSG mice engrafted with the human myeloma cell lines NCI-H929 or MM.1S engineered to express GFP and firefly luciferase (NCI-H929.GFP.ffluc and MM.1S.GFP.ffluc). As previous in vitro results showed that constitutive CMR signaling was sufficient to improve tumor cell killing and T cell expansion, in vivo tumor control in a myeloma model lacking CSF1 was first investigated. NCI-H929.GFP.ffluc myeloma cells were engrafted into NSG mice, and a single CAR T cell dose was injected at day 10, when tumors were established (FIG. 12a). Coexpression of CMR only led to minor but not significant improvement of anti-tumor function and mouse survival by mζ.CMR T cells (FIGS. 12b-d). To investigate the impact of CSF1 on the anti-tumor function of mζ.CMR T cells, NSG mice were engrafted with NCI-H929.GFP.ffluc cells engineered to secrete CSF1 (NCI-H929_{CSF1}.GFP.ffluc). As NCI-H929_{CSF1}.GFP.ffluc cells show delayed engraftment, ACT was performed on day 21, when tumors were established, compared to day 10 for NCI-H929.GFP.ffluc engrafted mice (FIG. 12e). The control conditions, mA and CMR alone, did not impact tumor growth or mouse survival compared to untreated mice, thus, excluding the option of unspecific tumor rejection (FIGS. 12f-g). While no anti-tumor activity was detected in mice treated with mζ T cells, mζ.CMR T cell treatment led to significant tumor control starting from day 32 (mζ vs. mζ.CMR: PD2=0.0093, P_{D40}=0.0017) (FIGS. 12g-h). All mice treated with mζ.CMR T cells survived until the end of the experiment (day 100) compared to a median survival of 52 days post tumor engraftment in the m treated group (mζ vs. mζ.CMR: P=0.024, FIG. 12h).

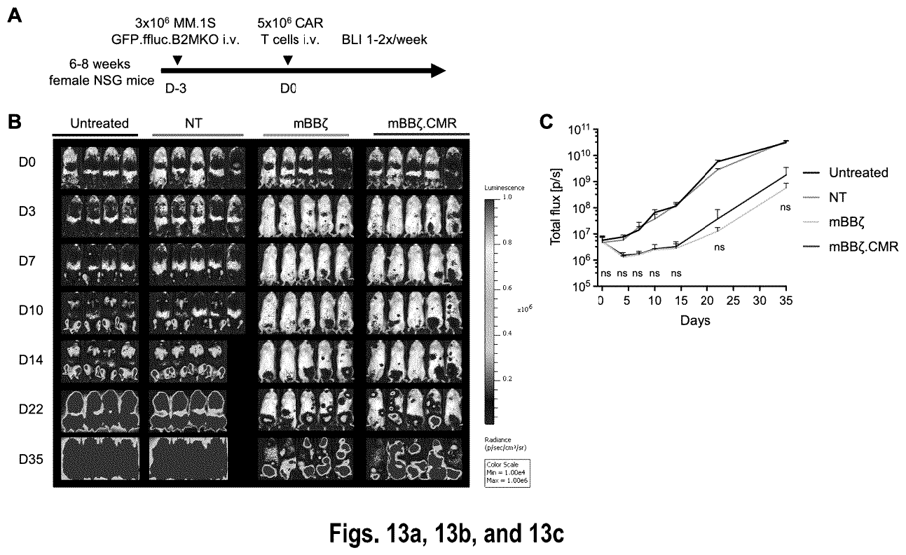

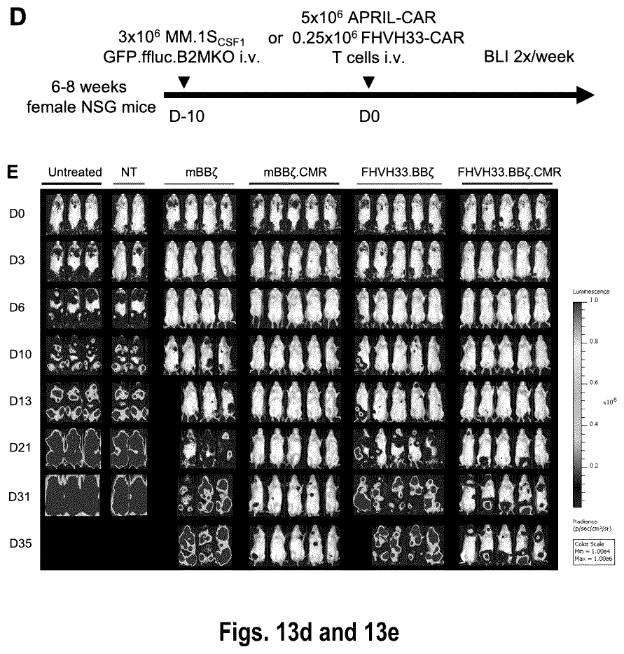

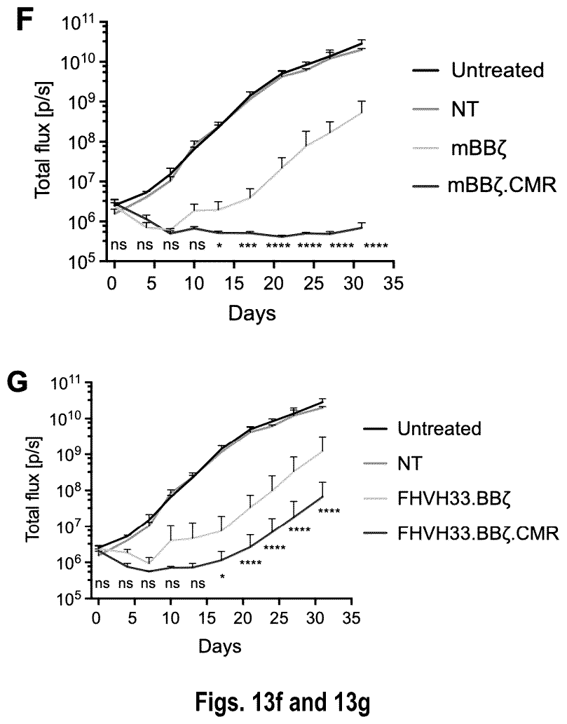

Next, whether CMR coexpression could also improve tumor control in mice engrafted with MM.1S.GFP.ffluc cells was tested. To avoid T cell receptor (TCR) mediated rejection of tumor cells in vivo, a β₂ microglobulin (B2M) knockout cell line (MM.1S.GFP.ffMuc.B2MKO) and one which secretes CSF1 (MM.1S_{CSF1}.GFP.ffluc.B2MKO) were generated. NSG mice engrafted with MM.1S_{CSF1}.GFP.ffluc.B2MKO myeloma cells were treated with a single CAR T cell infusion at day 3 (FIG. 13a). mBBζ and mBBζ.CMR T cells produced similar anti-tumor functions against MM.1S.GFP.ffluc.B2MKO cells indicating that the baseline constitutive CMR activity alone was not sufficient to enhance tumor control in vivo (FIGS. 13b-c). To test whether additional CMR signaling via CSF1 could enhance anti-tumor function of mBBζCMR T cells, the mouse model was switched to MM.1S_{CSF1}.GFP.ffluc.B2MKO cells. Due to delayed tumor growth kinetics, ACT was performed on day 10, compared to day 3 for MM.1S.GFP.ffluc.B2MKO engrafted mice (FIG. 13d). As observed before, treatment with mBBζ T cells led to similar anti-tumor function for about two weeks, followed by relapse and rapid tumor outgrowth. On the contrary, treatment with mBBζ.CMR T cells led to a sustained tumor control which lasted for over 30 days post ACT and was significantly better compared to mBBζ (mBBζ vs. mBBζ.CMR: P_{D13}=0.0208, P_{D17}=0.0003, P_{D21-31}<0.0001, (FIGS. 13e-f). To confirm the findings with a different CAR targeting exclusively, BCMA, the published fully human heavy-chain-only (FHVH33) CAR was tested alone (FHVH33.BBQ or in combination with CMR (FHVH33.BBζ.CMR) (Lam N, et al. Nat Commun. 2020; 11(1):283.). As shown in FIGS. 13e and 13g, CMR coexpression led to a notable enhancement of anti-tumor function (FHVH33.BBC vs. FHVH33.BBζ.CMR: PD17=0.0256, P_{D21-31}<0.0001 (FIGS. 13e and 13g). Thus, it was demonstrated that CMR coexpression can greatly improve the in vivo myeloma control of CAR T cells in two different myeloma xenograft models that provide CSF1-rich environments. This improvement was found in combination with CARs harboring different signaling and antigen-binding domains.

### Example 6

### Assessment of Serial Killing Capacity and Expansion of TCR+VMR and TCR+CMR T Cells Compared to TCR T Cells

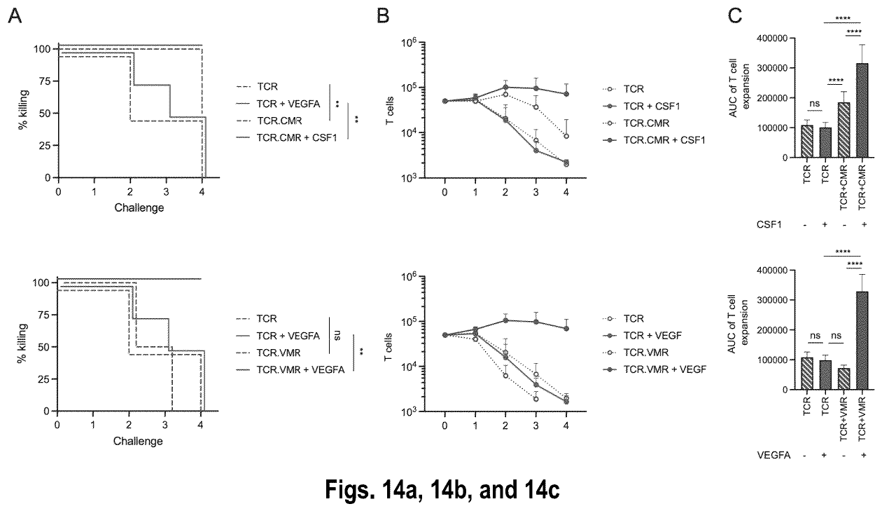

As co-expression of the biosensors VMR and CMR enhanced overall antitumor function and expansion of CAR T cells, it was hypothesized that transgenic TCR-based adoptive T cell therapy can also benefit from biosensor co-expression. To determine the functional benefit of biosensor co-expression in transgenic TCR T cells, CD8+ T cells were transduced with the HLA-A*02:01-restricted survivin-specific TCR without (TCR) or with biosensors (TCR.VMR and TCR.CMR) (C. Arber, et al., J. Clin. Invest. 125, 157-168 (2015)). A sequential coculture assay of TCR, TCR.VMR, and TCR was performed.CMR with target leukemia cell line BV173 at an effector:target ratio of 1:5 in media containing no cytokines, 25 ng/ml VEGF or 10 ng/ml CSF1. Addition of VEGF or CSF1 had no significant impact on tumor cell killing in TCR T cells which stopped killing on average after 2-3 tumor challenges (FIG. 14a). However, in VEGF or CSF1 containing media TCR.VMR and TCR.CMR T cells maintained superior killing capacity throughout all challenges (TCR_{VEGFA} vs TCR.VMR_{VEGFA} P<0.009, TCR_{CSF1} vs TCR.CMR_{CSF1} P<0.009, FIG. 14a). It was found that TCR.VMR T cells require VEGF for enhanced antitumor function while TCR.CMR T cells show a trend towards improved tumor killing also in the absence of CSF1 which was further augmented through CSF1 addition. Improved anti-tumor function of TCR.VMR and TCR.CMR T cells was accompanied by enhanced expansion in the presence of respective target ligands (TCR_{VEGF} vs TCR.VMR_{VEGFA} P<0.0001, TCR_{CSF1} vs. TCR.CMR_{CSF1} P<0.0001, FIGS. 14b and 14c). Also, constitutive CMR activity lead to significantly enhanced expansion of TCR.CMR T cells in the absence of CSF1 which was further elevated upon addition of CSF1 (TCR.CMR_{media} vs TCR.CMR_{CSF1} P<0.0001, FIGS. 14b and 14c).

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1-46. (canceled)

47. A method of constructing a chimeric receptor, comprising:
(a) selecting an extracellular ligand-binding domain and an intracellular signaling domain for the chimeric receptor from a plurality of extracellular ligand-binding domains and intracellular signaling domains based on a predetermined input signal and output function to be coupled by the chimeric receptor;
(b) selecting a transmembrane domain from a plurality of transmembrane domains for linking the extracellular ligand-binding and the intracellular signaling domain;
(c) self-associating three-dimensional structures of the extracellular ligand-binding domain, the intracellular signaling domain, and the transmembrane domain by docking;
(d) linking and assembling subunit structures into an ensemble of chimeric receptor structures;
(e) energy minimizing the oligomeric receptor models and selecting a subset of energy-minimized oligomeric receptor models having low energy by clustering the energy-minimized oligomeric receptor models based on an energy function;
(f) calculating stability of the subset of the energy-minimized oligomeric receptor models upon binding to the input signal, wherein a level of stability corresponds to a degree of ligand-induced oligomerization of the chimeric receptor;
(g) calculating a level of long-range mechanical dynamic coupling between the extracellular ligand-binding domain and the intracellular signaling domain of the subset of the energy-minimized oligomeric receptor models;
(h) ranking the subset of the energy-minimized oligomeric receptor models based on the level of stability thereof and the level of long-range mechanical dynamic coupling between the extracellular ligand-binding domain and the intracellular signaling domain; and
(i) selecting an optimal oligomeric receptor model with specific levels of stability and/or long-range mechanical dynamic coupling between the extracellular ligand-binding domain and the intracellular signaling domain.

48. The method of claim 47, further comprising repeating step (a) to step (i) for a plurality of combinations of extracellular ligand-binding domains, intracellular signaling domains, and transmembrane domains; and selecting an optimal oligomeric receptor model from the plurality of combinations with specific levels of stability and/or levels of long-range mechanical dynamic coupling between the extracellular ligand-binding domain and the intracellular signaling domain.

49. The method of claim 47, further comprising, prior to step (e), de novo designing a linker connecting the extracellular ligand-binding domain and the transmembrane domain or a linker connecting the transmembrane domain and the intracellular signaling domain.

50. The method of claim 47, wherein step (e) comprises energy minimizing the oligomeric receptor models by a Monte Carlo Minimization with simulated annealing.

51. The method of claim 47, wherein step (g) comprises calculating the level of long-range mechanical dynamic coupling between the extracellular ligand-binding domain and the intracellular signaling domain using a Rosetta force field and an Elastic Network model.

52. The method of claim 47, wherein step (g) comprises self-associating the transmembrane domain using EFDOCK-TM.

53. The method of claim 47, wherein the input signal is present in a tumor microenvironment associated with cancer.

54. The method of claim 53, wherein the cancer comprises a solid tumor.

55. The method of claim 47, wherein the output function comprises an anti-tumor function.

56. The method of claim 55, wherein the anti-tumor function comprises: (i) secretion of one or more cytokines from an immune cell, (ii) co-stimulation of the immune cell, (iii) cell survival of the immune cell, (iv) proliferation of the immune cell, (v) migration of the immune cell, (vi) functionality of the immune cell, or a combination thereof.

57. The method of claim 47, wherein the input signal comprises a growth factor, cytokine, or interleukin.

58. The method of claim 57, wherein the input signal comprises vascular endothelial growth factor (VEGF), basic fibroblast growth factor (bFGF), platelet-derived growth factor (PDGF), hepatocyte growth factor (HGF), insulin-like growth factor (IGF), interleukin-8 (IL-8), TGFβ, IL-10, or colony stimulating factor 1 (CSF-1), interleukin-34 (IL-34), stem cell factor (SCF), interleukin-9 (IL-9), interleukin-4 (IL-4), interleukin-6 (IL-6), interleukin-10 (IL-10), Angiopoietin-1 (Ang1), or CD202), Thrombopoietin (TPO), Osteopontin (OPN), Receptor activator of nuclear factor kappa beta (NFkB) ligand, RANK ligand (RANKL), Fibroblast growth factor (FGF-1, -2), Vascular cell adhesion protein 1 (VCAM-1), Notch ligands: Jagged1, Jagged2, Delta-like1, Delta-like3, Delta-like4, GM-SCF/CSF2, G-CSF/CSF3, IL-1b, MMP-1, MMP-2, MMP-3, MMP-7, MMP-8, MMP-9, MMP-13, MMP-14, TIMP-1, TIMP-2, TIMP-3, TIMP-4, PGD2, PGE2, PGF2α, PGI2, TXA2, PGH2, BMP1, BMP2, BMP3, BMP4, BMP5, BMP6, BMP7, BMP8, BMP9, BMP10, BMP11, BMP12, BMP13, BMP14, BMP15, N6-Cyclopentyladenosine, N6-3-methoxyl-4-hydroxybenzyl adenine riboside (B2), adenosine, CCPA, Certain Benzodiazepines and Barbiturates, 2′-MeCCPA, GR 79236, SDZ WAG 994, Benzyloxy-cyclopentyladenosine (BnOCPA), N6-3-methoxyl-4-hydroxybenzyl adenine riboside (B2), ATL-146e, CGS-21680, Regadenoson, adenosine, 5′-N-ethylcarboxamidoadenosine, BAY 60-6583, adenosine, LUF-5835, LUF-5845, 2-(1-Hexynyl)-N-methyladenosine, CF-101 (IB-MECA), Adenosine, 2-Cl-IB-MECA, CP-532,903, or MRS-3558.

59. The method of claim 58, wherein the input signal comprises vascular endothelial growth factor (VEGF).

60. The method of claim 47, wherein the extracellular ligand-binding domain comprises one or more extracellular domains of VEGFR2, VEGFR1, FGFR1, PDGFR, HGFR, IGFR, IL-8R, TGFβR1, IL-10R, CSF1R, SCFRKIT, cKIT, CD117, IL-9R, IL-4R, IL-6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-1R, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-1A, ActR-2A, ActR-2B, or a variant or fragment thereof.

61. The method of claim 47, wherein the extracellular ligand-binding domain comprises D1-7; D1-4 and D7; or D1-3 extracellular domains of the VEGFR2.

62. The method of claim 47, wherein the transmembrane domain comprises a transmembrane domain from a protein selected from VEGFR2, VEGFR1, FGFR1, PDGFR, HGFR, IGFR, IL-8R, TGFβR1, IL-10R, CSF1R, SCFR, KIT, cKIT, CD 117, IL-9R, IL-4R, IL-6R, IL-10R, Tie2, CD202, C-MPL, TPOR, CD44, RANK, FGFR1, FGFR2, FGFR3, FGFR4, VLA-4, NOTCH1, NOTCH2, NOTCH3, NOTCH4, GM-SCFR, CSF2R, G-CSFR, CSF3R, IL-1R, PD-1, PDL1, PDL2, CTLA-4, CD200R TIM3, LAG-3, 2B4, BTLA, CTLA4, TIM3, LAG3, PD1, TIGIT, LAIR1, FASLG, ID2, CD80, CD83, KLRG1, KLRD1, KLRC1, B7-1, B7-H1, CD160, EP1, EP2, EP3, EP4, IP, TP, DP1, DP2, FP, A1, A2A, A2B, A3, BMPR1A, BMPR1B, BMPR2, ActR-1A, ActR-2A, ActR-2B, CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF1, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

63. The method of claim 47, wherein the transmembrane domain comprises a transmembrane domain of a c-MPL receptor.

64. The method of claim 47, wherein the intracellular signaling domain comprises an intracellular domain of a protein selected from CSF-1R, Kit, TIE3, DAP12, DAP10, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, TLR, TLR5, Myd88, lymphocyte receptor chain, IgE, IgG, CD16α, FcγRIII, FcγRII, CD28, 4-1BB, CD4, CD8, IL-2R, IL-7R, IL-10R, IL-12R, IL-15R, IL-18R, IL-23R, EpoR, CD27, CD28, ICOS, HVEM, LIGHT, CD40L, CD27, OX40, DR3, GITR, CD30, SLAM, CD2, 2B4, CD226, TRAF1, TRAF2, TRAF3, CD40BP, TRAF3IP1, TRAF4, TRAF7, TRAP1, TNFR1, TNFRSF1A, CD120a, TRAP100, MED24, TNFR2, TNFRSF1B, CD120b, LTBR, TNFRSF3, OX40, TNFRSF4, CD134, CD40, TNFRSF5, DcR3, TNFRSF6B, CD27, TNFRSF7, CD30, TNFRSF8, 4-1BB, TNFRSF9, CD137, TRAIL R1, CD261, TNFRSF10A, TRAIL R2, CD262, TNFRSF10B, TRAILR3, TNFRSF10C, TRAIL R4, CD264, TNFRSF10D, TNFRSF11A, Osteoprotegerin, TNFRSF11B, TNFRSF12A, FN14, TWEAKR, TACI, TNFRSF13B(CD267), BAFFR, TNFRSF13C, CD268, HVEM, TNFRSF14, CD270, BCMA, TNFRSF17, CD269, GITR, TNFRSF18, CD357, RELT, TNFRSF19L, TNFRSF19, TROY, TNFRSF21, DR6, TNFRSF25, DR3, TNFRSF12, and a variant or fragment thereof.

65. The method of claim 47, wherein the intracellular signaling domain comprises an intracellular domain of a cytokine receptor.

66. The method of claim 47, wherein the intracellular signaling domain comprises an intracellular domain of a c-MPL receptor.
